(19) United States
(12) Patent Application Publication
(10) Pub. No.: US 2022/0409070 A1
(43) Pub. Date: Dec. 29, 2022

(54) PROXIMITY SENSOR CIRCUITS AND RELATED SENSING METHODS

(71) Applicant: PyrAmes Inc., Cupertino, CA (US)

(72) Inventors: Xina QUAN, Saratoga, CA (US); Weyland LEONG, Daly City, CA (US); Junjun LIU, Cupertino, CA (US); Arthur MUIR, San Mateo, CA (US); Thomas ROXLO, Saratoga, CA (US); Siddharth SIDDHARTH, Santa Clara, CA (US); Alan WALENDOWSKI, San Jose, CA (US); William SUTHERLAND, Los Gatos, CA (US)

(73) Assignee: PyrAmes Inc., Cupertino, CA (US)

(21) Appl. No.: 17/754,391

(22) PCT Filed: Oct. 3, 2020
(86) PCT No.: PCT/US2020/054178

§ 371 Date: Mar. 31, 2022

Related U.S. Application Data
(60) Provisional application No. 62/910,125, filed on Oct. 3, 2019.

(51) Int. Cl. A61B 5/021; G01D 5/24; A61B 5/00

(52) U.S. Cl. CPC A61B 5/02108; G01D 5/24; A61B 5/7221; A61B 5/681; A61B 5/7207

(57) ABSTRACT

Disclosed are one or more proximity sensors. At least one of the proximity sensors includes a first dielectric layer, an electrically conductive layer, and an electrode. The first dielectric layer includes an inner surface and an outer surface. The electrically conductive layer is positioned proximate to one of the inner surface or the outer surface of the first dielectric layer. The electrode includes an outer surface. The outer surface of the electrode is positioned proximate the inner surface of the first dielectric layer. The outer surface of the electrode and the electrically conductive layer define a gap.

### CROSS-REFERENCE TO RELATED APPLICATIONS

The present application claims priority under 35 U.S.C. § 119(e) to U.S. Provisional Patent Application Ser. No. 62/910,125, titled PROXIMITY SENSOR CIRCUITS AND RELATED SENSING METHODS, filed Oct. 3, 2019, the disclosure of which is herein incorporated by reference in its entirety.

### TECHNICAL FIELD

The present disclosure generally relates to proximity sensors and related sensing methods for sensing hemodynamic changes (or pulse-waveforms) of a user.

### SUMMARY

In one general aspect, the present disclosure provides a proximity sensor. The proximity sensor comprises a first dielectric layer, an electrically conductive layer, and an electrode. The first dielectric layer comprises an inner surface and an outer surface. The electrically conductive layer is positioned proximate to one of the inner surface or the outer surface of the first dielectric layer. The electrode comprises an outer surface. The outer surface of the electrode is positioned proximate the inner surface of the first dielectric layer. The outer surface of the electrode and the electrically conductive layer define a gap.

In another aspect, the proximity sensor further comprises a foam layer.

In another aspect, the proximity sensor further comprises a sealant layer disposed over the sensing surface.

In another aspect of the proximity sensor, the electrically conductive layer is positioned proximate the inner surface of the first dielectric layer, and the proximity sensor further comprises a second dielectric layer disposed between the electrode and the electrically conductive layer, wherein the outer surface of the electrode and the electrically conductive layer define a gap.

In another aspect of the proximity sensor, the second dielectric layer has a thickness less than 3 μm.

In another aspect of the proximity sensor, the second dielectric layer has a textured surface.

In one general aspect, the present disclosure provides a proximity sensor. The proximity sensor comprises a first dielectric layer, an electrically conductive layer, a sensing electrode, and a reference electrode. The first dielectric layer comprises an inner surface and an outer surface. The electrically conductive layer is positioned proximate to one of the inner surface or the outer surface of the first dielectric layer. The sensing electrode is positioned proximate the inner surface of the first dielectric layer. The sensing electrode comprises an inner surface and an outer surface. The outer surface of the sensing electrode is positioned proximate the inner surface of the first dielectric layer. The outer surface of the sensing electrode and the electrically conductive layer define a gap. The reference electrode is disposed relative to the sensing electrode. The reference electrode is positioned proximate the inner surface of the first dielectric layer. The reference electrode comprises an inner surface and an outer surface. The outer surface of the reference electrode is positioned proximate the inner surface of the first dielectric layer. The outer surface of the reference electrode and the electrically conductive layer define a gap.

In another aspect, the reference electrode is disposed laterally relative to the sensing electrode, stacked relative to the sensing electrode, or mechanically isolated from the sensing electrode.

In another aspect, the proximity sensor further comprises a fifth dielectric layer disposed between the reference electrode and the first dielectric layer.

In another aspect, the proximity sensor further comprises a sixth dielectric layer disposed between the sensing electrode and the first dielectric layer.

In another aspect, the proximity sensor further comprises a foam layer, wherein the sensing electrode and the reference electrode are positioned on opposite sides of the foam layer.

In one general aspect, the present disclosure provides a proximity sensor module. The proximity sensor module comprises a sensor element substrate, at least one electrically conductive electrode, an electronics module, and at least one electrically conductive pad, and at least one elastically-deformable electrically-conductive feature. The sensor element substrate comprises a proximity sensors described in the present disclosure. The at least one electrically conductive electrode lead is disposed on the sensor element substrate. The at least one elastically-deformable electrically-conductive feature is disposed on the at least one electrically conductive electrode lead or on at least one electrically conductive pad. The at least one electrically conductive pad is disposed on the electronics module. The at least one electrically conductive pad positioned to make an electrical connection between the at least one electrically conductive lead and the at least one electrically conductive pad through the at least one elastically-deformable electrically-conductive feature.

In one general aspect, the present disclosure provides a circuit for measuring physiological parameters. The circuit comprises a sensor circuit, a transducer circuit coupled to the sensor circuit, and a signal-sensing circuit. The sensor circuit comprises a sensor element substrate comprising any one of the proximity sensors described in the present disclosure. The sensor element comprises at least one electrode. The sensor circuit is configured to monitor a capacitance signal between the at least one electrode and the skin of a user. The capacitance signal represents motion, pressure and/or electric field modulations attributable to pulse-wave events or to changes in pressure or blood flow in blood vessels of the user or to movement of parts of the body of the user. The transducer circuit is coupled to the sensor circuit. The transducer circuit is configured to convert the monitored capacitance signal into a digital signal indicative of the monitored capacitance signal. The signal-sensing circuit is configured to receive the digital signal and determine at least one physiological parameter associated with the user.

In another aspect of the circuit, the physiological parameters comprise blood pressure, systolic, diastolic, mean arterial pressure, pulse pressure, respiration rate, or combinations thereof, and their variabilities, both as time series values and as trends.

In another aspect of the circuit, the signal-sensing circuit is configured to provide quality ratings for subsequent sensor data to filter sensor data for use to extract blood pressure values or to estimate a confidence level for the extracted values.

In one general aspect, the present disclosure provides a circuit for measuring physiological parameters. The circuit comprises a sensor circuit, a transducer circuit coupled to the sensor circuit, and a signal-sensing circuit. The sensor circuit comprises a sensor element substrate comprising any one of the proximity sensors described in the present disclosure. The sensor circuit comprises at least one electrode. The sensor circuit is configured to monitor a capacitance signal between the at least one electrode and the skin of a user. The capacitance signal represents motion, pressure and/or electric field modulations attributable to pulse-wave events, to changes in pressure or blood flow in blood vessels of the user, or to movements of parts of the body of the user. The transducer circuit coupled to the sensor circuit, wherein the transducer circuit is configured to convert the monitored capacitance signal into a digital signal indicative of the monitored capacitance signal. The signal-sensing circuit configured to implement blood pressure and other hemodynamic and physiological models.

In another aspect of the circuit, the signal-sensing circuit is configured to convert the capacitance signal to a format that can be displayed on an external monitor and/or processed and stored on an external data system.

In another aspect of the circuit, the signal-sensing circuit is configured to employ input obtained from a prescribed start-up regimen where the sensor is applied and then used in multiple positions.

In one general aspect, the present disclosure provides a method for hemodynamic monitoring via a wearable apparatus. The wearable apparatus comprises a sensor circuit comprising at least one electrode, a transducer circuit to receive signals from the sensor circuit and to convert the signals to digital signals and provide the digital signals to a signal-sensing circuit to process the digital signals. The method comprises sensing, by the sensor circuit, capacitance signals by the at least one electrode. The capacitance signals are representative of pressure and/or electric field modulations attributable to the pulse-wave events or to the changes in pressure or blood flow in the blood vessels of a user. The method further comprises converting, by the transducer circuit, the sensed capacitance signals into a digital signal indicative of the sensed capacitance signals, providing, by the transducer circuit, the digital signal to the signal-sensing circuit, processing, by the signal-sensing circuit, the digital signals representative of the changes in capacitance over time to generate a pulse-waveform data, correlating, by the signal-sensing circuit, the pulse-waveform data with various hemodynamic parameters, processing, by the signal-sensing circuit, the pulse-waveform data, and determining, by the signal-sensing circuit, a hemodynamic parameter based on the pulse-waveform data.

In another aspect, the method further comprises reducing motion artifacts with an accessory device.

The above discussion/summary is not intended to describe each aspect or every implementation of the present disclosure. The figures and detailed description that follow also exemplify various aspects.

### BRIEF DESCRIPTION OF THE FIGURES

Various example aspects may be more completely understood in consideration of the following detailed description in connection with the accompanying drawings, in which:

FIG. 1 shows an example proximity sensor having a free floating foil construction with a dielectric layer separating a sensing electrode from an electrically conductive layer, in accordance with at least one aspect of the present disclosure;

FIG. 2 shows an example proximity sensor having a free floating foil construction with a separate dielectric layer separating a sensing electrode from an electrically conductive layer to control the distance between the electrically conductive layer and the sensing electrode, in accordance with at least one aspect of the present disclosure;

FIG. 3 shows an example proximity sensor having a free floating foil construction with an adhesive layer formed around sensing electrode elements or around an entire sensing electrode array, in accordance with at least one aspect of the present disclosure;

FIG. 4 shows an example proximity sensor having a free floating foil construction with dielectric, foam, or double-sided tape disposed over sensing electrode lead(s), in accordance with at least one aspect of the present disclosure;

FIG. 5 shows an example proximity sensor having a free floating foil construction with a reference electrode and a sensing electrode, in accordance with at least one aspect of the present disclosure;

FIG. 6 shows an example proximity sensor having a free floating foil construction with a layer of dielectric material attached or coated onto a reference electrode which is significantly thicker and/or has a significantly lower dielectric constant than the material used with the sensing electrode, in accordance with at least one aspect of the present disclosure;

FIG. 7 shows an example proximity sensor having a free floating foil construction with a foam layer disposed between sensor elements and a mounting structure such as a wristband to provide conformity and ensure that both reference elements and sensing elements have similar contact to the skin, in accordance with at least one aspect of the present disclosure:

FIG. 8 shows an example proximity sensor having a free floating foil construction with reference electrodes located on an opposite side of a foam substrate layer from sensing electrodes, in accordance with at least one aspect of the present disclosure;

FIG. 9 shows one view of an example attachment structure for a proximity sensor having a free floating foil construction, where the attachment structure includes a number of materials used for a band, patch, or other method to fasten the sensor array to the skin, in accordance with at least one aspect of the present disclosure;

FIG. 10 shows a section view of the example attachment structure shown in FIG. 9, taken along section line 10-10, in accordance with at least one aspect of the present disclosure;

FIG. 11 shows a detail view of the section view of the example attachment structure shown in FIG. 10, taken along line 11, in accordance with at least one aspect of the present disclosure;

FIG. 12 shows an example of re-engageable contacts between an electronics module and sensor/electrode leads of a proximity sensor having printed conductive elastomer conductive bumps for elastically compressible re-engageable contacts, in accordance with at least one aspect of the present disclosure;

FIG. 13 shows an example method of printing conductive elastomer bumps for elastically compressible re-engageable contacts, in accordance with at least one aspect of the present disclosure:

FIG. 14 shows an example of conductive elastomer bumps printed on electrode leads to be pressed against an electronics module, in accordance with at least one aspect of the present disclosure:

FIG. 15 shows an example of conductive elastomer bumps fabricated by embossing structures into a substrate that supports the electrodes, in accordance with at least one aspect of the present disclosure;

FIG. 16 shows an example of conductive elastomer bumps fabricated by mechanically deforming electrical leads, in accordance with at least one aspect of the present disclosure;

FIG. 17 shows an example method of forming connections between an electronics module and a sensor array by mechanically deforming electrical leads, in accordance with at least one aspect of the present disclosure;

FIG. 18 shows an example connector formed by the method described in FIG. 17 having mechanically isolated individual electrode leads in an array of electrode leads with improved compliance, in accordance with at least one aspect of the present disclosure;

FIG. 19 shows an example connector formed by the method described in FIG. 17 having mechanically rigid spring fingers optionally supported and/or deformed with foam or other spacer material, in accordance with at least one aspect of the present disclosure:

FIG. 20 shows an example of mating contacts on the electronics module used in conjunction with the connector shown in FIG. 19, in accordance with at least one aspect of the present disclosure;

FIG. 21 shows an example band for adults adjustably sized to fit radial, brachial, tibial, dorsal, and/or femoral pulse points, the band including reusable electronics can be utilized with disposable sensor(s) through the use of a sealed or partially sealed electronics module that snaps into a tray, a multi-part case is assembled around the electronics and fastened through known fastening methods, in accordance with at least one aspect of the present disclosure:

FIG. 22 shows a section view of the band for adults shown in FIG. 21, in accordance with at least one aspect of the present disclosure;

FIG. 23 shows an example band for infants adjustably sized to fit radial, brachial, tibial, dorsal, and/or femoral pulse points, in accordance with at least one aspect of the present disclosure;

FIG. 24 shows a section view of the band for infants shown in FIG. 22, in accordance with at least one aspect of the present disclosure;

FIG. 25 shows a block diagram of the electronics, in accordance with at least one aspect of the present disclosure;

FIGS. 26A-26B show examples of sensor circuits and sensing signal circuits, in accordance with at least one aspect of the present disclosure;

FIGS. 27A-27D illustrate an example of an apparatus and the resulting interaction with the skin of a user, in accordance with at least one aspect of the present disclosure:

FIG. 28 is a block diagram that exemplifies an example way for implementing the electronics and/or signal flow from the apparatus, in accordance with at least one aspect of the present disclosure;

FIGS. 29A-29B illustrate various example apparatus, in accordance with at least one aspect of the present disclosure;

FIGS. 30A-30B illustrate an example apparatus having a packaged array of sensors including a plurality (e.g., four) of electrodes having different capacitive sensitivities, in accordance with at least one aspect of the present disclosure;

FIGS. 31A-31C illustrate an apparatus, in accordance with at least one aspect of the present disclosure:

FIGS. 32A-32C illustrate example data collected using an apparatus and data collected using an arterial line, in accordance with various experimental aspects;

FIGS. 33A-33C illustrate example data collected using an apparatus and collected using an arterial line, in accordance with various experimental aspects; and

FIGS. 34A-34C illustrate an example of changes in heart rate and blood pressure as collected using an apparatus and collected using an arterial line, in accordance with various experimental aspects.

FIG. 35 is a graph of systolic blood pressure (sBP) calculated from sensor data versus arterial line sBP, in accordance with various experimental aspects.

FIG. 36 is a graph of systolic blood pressure (sBP) versus elapsed time, in accordance with various experimental aspects.

FIG. 37 illustrates a method for hemodynamic monitoring, in accordance with at least one aspect of the present disclosure.

FIGS. 38A-38D illustrates a method for measuring and processing one or more physiological parameters, in accordance with at least one aspect of the present disclosure.

FIGS. 39A-39C illustrates a method for measuring and processing one or more physiological parameters, in accordance with at least one aspect of the present disclosure.

While various aspects discussed herein are amenable to modifications and alternative forms, aspects thereof have been shown by way of example in the drawings and will be described in detail. It should be understood, however, that the intention is not to limit the disclosure to the particular aspects described. On the contrary, the intention is to cover all modifications, equivalents, and alternatives falling within the scope of the disclosure including aspects defined in the claims. In addition, the term “example” as used throughout this application is only by way of illustration, and not limitation.

### DESCRIPTION

Before explaining various forms of proximity sensor circuits, electrical-signal sensing circuit, signal processing circuits, and related sensing methods in detail, it should be noted that the illustrative forms are not limited in application or use to the details of construction, dimensions, and arrangement of parts illustrated in the accompanying drawings and description. The illustrative forms may be implemented or incorporated in other forms, variations and modifications, and may be practiced or carried out in various ways. Further, unless otherwise indicated, the terms and expressions utilized herein have been chosen for the purpose of describing the illustrative forms for the convenience of the reader and are not for the purpose of limitation thereof.

Further, it is understood that any one or more of the following-described forms, expressions of forms, examples, can be combined with any one or more of the other following-described forms, expressions of forms, and examples.

In the following discussion, various implementations and applications are disclosed to provide an understanding of the instant disclosure by way of non-limiting examples.

In certain examples, aspects of the present disclosure involve one or more sensor circuits configured and arranged to sense hemodynamic changes (or pulse-waveforms) of a user with the sensor circuit configured in a manner to monitor the physiologic changes of the user by using a single electrode placed near/onto a surface to be measured. These and other aspects employ the sensor circuit configured to sense the hemodynamic changes consistent with one more of the below-described aspects and/or mechanisms.

More specific example aspects are directed to an apparatus having at least one sensor circuit, the sensor circuit including an electrode, and an electrical-signal sensing circuit. The apparatus can be used to monitor one or more of the hemodynamic parameters in a non-invasive manner and in real-time. For example, the electrical-signal sensing circuit can sense pulse-wave events, while the sensor circuit is placed near or onto skin, by monitoring capacitance changes. The capacitance changes carried by the electrode are responsive to pressure and/or electric field modulations attributable to the pulse-wave events or to the changes in pressure or blood flow in the blood vessels (e.g., hemodynamics). The electrode can be used to determine capacitance changes between the electrode and the skin of the user. The sensor circuit including the electrode can be arranged with a transducer circuit, which is used to provide an electrical signal to the electrical-signal sensing circuit indicative of the changes in capacitance and/or pressure. Due to pulse-wave events, the distance between the skin of the user and the electrode can change and/or the electric field distribution around the blood vessels can change, resulting in a relative change in capacitance as measured using the sensor circuit. The changes in capacitance over time can be processed by the electrical-signal sensing circuit and used to generate and/or determine a pulse-waveform. In various aspects, the pulse-waveform is correlated with various hemodynamic parameters. As specific examples, the pulse-waveform can be processed to determine a heart rate, blood pressure, arterial stiffness, and/or blood volume. Machine learning algorithms can be used to derive hemodynamic parameters from the shape of the pulse waveform.

The electrode can be in contact with the skin of the user and/or in proximity. In some aspects, the electrode is constrained onto (whether in contact or not) the user using a mechanical constraint (e.g., a wristband, an elastically compliant band, or an article of clothing) and/or an adhesive. The electrode can be located near a blood vessel, preferably near a palpable pulse point such as but not limited to the radial, brachial, carotid, tibial, dorsal and temporal pulse points.

In other specific aspects, the apparatus includes a plurality of electrodes. For example, the apparatus can include a plurality of sensor circuits and each sensor circuit includes one of the plurality of electrodes. The plurality of electrodes can be arranged as part of a transducer circuit, which is used to provide electrical signals (e.g., a digital) to the electrical-signal sensing circuit indicative of the changes in capacitance that are responsive to modulations in distance between the skin of the user and the electrode, pressure and/or electric field and attributable to hemodynamic or pulse-wave events. In various related aspects, the plurality of sensor circuits are mechanically separated and/or arranged in an array (e.g., a sensor array). Each of the sensor circuits can be constructed differently, such as having different geometries, dielectric layers, locations, sensitivities, among other constructions as further described herein.

Various aspects are directed to a method of using the above-described apparatus. The method can include placing at least one electrode of an apparatus near or onto the skin of the user and sensing pulse-wave events. The pulse-wave events can be sensed while the at least one electrode is placed near or onto the skin of a user, using an electrical-signal sensing circuit of the apparatus, by monitoring capacitance changes that are responsive to pressure and/or electric field modulations attributable to hemodynamic or pulse-wave events. The pulse-wave events can be used to generate a pulse-waveform and/or to determine various hemodynamic parameters. For example, the method can include determining diastolic blood pressure, systolic blood pressure, arterial stiffness, and/or blood volume using the pulse-wave events.

A specific method can include use of a flexible or bendable substrate of a wearable apparatus to secure the transducer circuit having at least one sensor circuit. The substrate supports and at least partially encloses the transducer circuit and the electrical-signal sensing circuit. The substrate further conforms to a portion of a user including blood vessels and locates the at least one electrode sufficiently close to the user's skin for electrically sensing hemodynamic or pulse-wave events via the capacitance changes, the changes in capacitance being responsive to pressure and/or electric field modulations attributable to hemodynamic or pulse-wave events. A transducer circuit converts the changes in capacitance into the electrical signals. The method further includes sensing the hemodynamic or pulse-wave events in response to the electrical signals from the transducer circuit via the electrical-signal sensing circuit and using a communication circuit, within or outside the wearable apparatus, to respond to the electrical-signal sensing circuit by sending hemodynamic-monitoring data to an external circuit.

Other aspects are directed to an apparatus for use as part of a wearable device characterized by a flexible or bendable substrate configured and arranged to support and at least partially enclose a transducer circuit and an electrical-signal sensing circuit and to conform to a portion of a user including blood vessels for hemodynamic monitoring. The apparatus includes the transducer circuit having at least one sensor circuit, the sensor circuit including an electrode, the electrical-signal sensing circuit, and a communication circuit, as previously described above.

### Hardware

Various example implementations of proximity sensor circuits and related sensing methods using sensor circuits configured and arranged to sense hemodynamic changes (or pulse-waveforms) of a user with the sensor circuit configured in a manner to monitor the physiologic changes of the user by using one or multiple electrodes placed near/onto a surface to be measured are described hereinbelow.

1. Free Floating Foil Construction to Improve Sensitivity

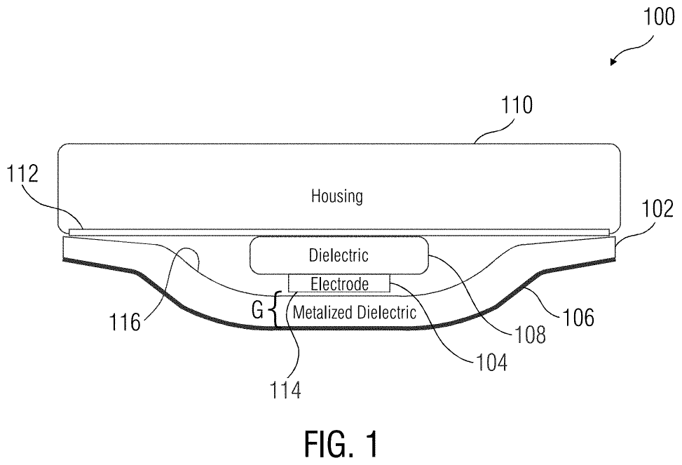

FIG. 1 shows an example proximity sensor 100 having a free floating foil construction with a first dielectric layer 102 separating a sensing electrode 104 from an electrically conductive layer 106, in accordance with at least one aspect of the present disclosure. In various aspects, the sensing electrode 104 may comprise multiple sensing elements or a sensing electrode array. In one aspect, the first dielectric layer 102 may be made of any suitable polymer or thin dielectric film, including but not limited to polyolefins, fluorinated polymers, polyurethanes, polyesters, silicones, polyamides, polyimides, parylenes, and glass, and in one aspect, it is made of Polyethylene terephthalate (PET). A second dielectric layer 108 couples the sensing electrode 104 to a housing 110. In the illustrated example, the second dielectric layer 108 is mounted to the housing 110 via an adhesive 112. The second dielectric layer 108 also may be made of a suitable polymer and in the illustrated aspect is made of PET having a thickness of 150 μm for example. In one aspect, the second dielectric layer 108 has a thickness of up to 150 μm preferably up to 50 μm and more preferably from 1 μm to 25 μm. The housing 110 may be made of low density polyethylene (LDPE) having a thickness in the range of 100 μm to 300 μm and preferably of 200 μm, for example.

The thin first dielectric layer 102 separates the sensing electrode 104 from the electrically conductive layer 106, which may be ungrounded (not connected to electronics circuit), grounded, or connected to an antenna to extend the antenna range. A distance G separates the surface 114 of the sensing electrode 104 from the electrically conductive layer 106. Optimal results are obtained when the first dielectric layer 102 is thick enough to provide some mechanical strength, elasticity and spring force to recover from a deformation, e.g. >1 μm>3 μm>5 μm>10 μm but thin enough to provide sensitivity, e.g. <25 μm<50 μm<150 μm. If the dielectric constant of the first dielectric layer 102 is sufficiently high, e.g. >5, >10, >50, >100, the first dielectric layer 102 can be thicker, up to 100-300 μm. In one aspect, the first dielectric layer 102 has a thickness of up to 150 μm preferably up to 50 μm and more preferably from 1 μm to 25 μm.

The first dielectric layer 102 may be a polymer film which has been metallized, e.g. through sputtering or other deposition/coating processes. The metallized film is particularly advantageous since the electrically conductive layer 106 is thin enough that it does not significantly affect the mechanical properties of the dielectric polymer film. The electrically conductive layer 106 may comprise thin metallic layers (e.g. aluminum foil, gold leaf, copper leaf, metallic hot or cold transfer films) that can also be used with a dielectric coating on one or both surfaces of the metallic layer or on the exposed surface of the sensing electrode 104. It is preferable that the dielectric coating of the first dielectric layer 102 is <1 μm<3 μm<5 μm<10 μm thick, without pinholes, and is not tacky or prone to surface blocking to avoid adhesion to the sensing electrode 104. The surface 114 of the sensing electrode 104 and/or the surface 116 of the first dielectric layer 102 or coating may be patterned or textured to reduce surface blocking.

Aluminum gold, silver, other metals, carbon, and conductive polymers can be used for the electrically conductive layer 106. The electrically conductive layer 106 and the sensing electrode 104 can also be printed from conductive inks. The thickness of the printed features will need to be controlled to preserve the sensitivity of the proximity sensor 100.

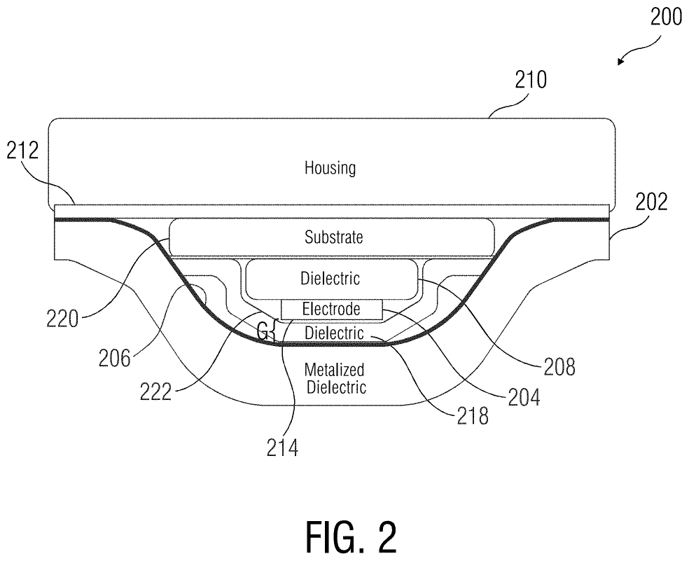

FIG. 2 shows an example proximity sensor 200 having a free floating foil construction with a first dielectric layer 218 separating a sensing electrode 204 from an electrically conductive layer 206 to control the distance G (e.g., gap) between an electrically conductive layer 206 and the sensing electrode 204, in accordance with at least one aspect of the present disclosure. In various aspects, the sensing electrode 204 may comprise multiple sensing elements or a sensing electrode array. In one aspect, the first dielectric layer 218 may be made of any suitable polymer and in one aspect, it is made of PET. The electrically conductive layer 206 may be formed on surface of a second dielectric layer 202, also made of PET, for example. A third dielectric layer 208 couples the sensing electrode 204 to a substrate 220 backing material, which is coupled to the housing 210 via an adhesive 212. The second and third dielectric layers 202, 208 also may be made of a suitable polymer and in the illustrated aspect are made of PET with the second dielectric layer 202 having a thickness of 12 μm for example, and the third dielectric layer 208 having a thickness of 150 μm for example. In one aspect, the second dielectric layer 208 has a thickness of up to 150 μm preferably up to 50 μm and more preferably from 1 μm to 25 μm. The housing 210 may be made of LDPE having a thickness in the range 100μ to 300μ and preferably of 200 μm, for example.

The distance G between the electrically conductive layer 206 and the sensing electrode 204 may be controlled with a separate dielectric layer shown here as the first dielectric layer 218 for manufacturing convenience or to ensure the electrically conductive layer 206 is embedded within the proximity sensor 200 packaging housing 210 and not susceptible to degradation due to exposure to environmental conditions. In one aspect, the first dielectric layer 218 can be a thin film or a coated or printed dielectric layer covering the surface 214 of the sensing electrode 204. The first dielectric layer 218 should avoid pinholes which could cause shorting between the electrodes through multiple connections to the electrically conductive layer 206. The first dielectric layer 218 alternatively can be a thicker layer if the dielectric constant is sufficiently high. The first dielectric layer 218 can be free floating or adhered to the sensing electrode 204 or to the substrate 220 backing material that supports the sensing electrode 204. The first dielectric layer 218 may have a thickness of less than 1 μm for example. A thin dielectric coating also may be provided on the surface of the electrically conductive layer 206 or on the exposed surface 214 of the sensing electrode 204. It is preferable that the first dielectric layer or coating is <0.1 μm<1 μm<3 μm<5 μm<10 μm thick, without pinholes, and is not tacky or prone to surface blocking to avoid adhesion to the sensing electrode 204. The surface 214 of the sensing electrode 204 and/or the surface 222 of the first dielectric layer 218 or coating may be patterned or textured to reduce surface blocking.

The sensing electrode 204 can be fastened with adhesive or other fastening method around each electrode element or around the entire electrode array to adhere to the first dielectric layer 218 or foil layer and to control buckling of the air gap between the sensing electrode 204 and the first dielectric layer 218 or foil layer.

The thin first dielectric layer 218 separates the sensing electrode 204 from the electrically conductive layer 206, which may be ungrounded (not connected to electronics circuit), grounded, or connected to an antenna to extend the antenna range. A distance G separates the surface 214 of the sensing electrode 204 from the electrically conductive layer 206. Optimal results are obtained when the first dielectric layer 218 is thick enough to provide mechanical strength, elasticity and spring force to recover from a deformation, e.g. >0.1 μm>1 μm>3 μm>5 μm>10 μm but thin enough to provide sensitivity, e.g. <25 μm<50 μm<150 μm. If the dielectric constant of the first dielectric layer 218 is sufficiently high, e.g. >5, >10, >50, >100, the first dielectric layer 218 can be thicker, up to 100-300 μm. In one aspect, the first dielectric layer 218 has a thickness of up to 150 μm preferably up to 50 μm and more preferably from 1 μm to 25 μm.

The first dielectric layer 218 may be a polymer film which has been metallized, e.g. through sputtering or other deposition/coating processes. The metallized film is particularly advantageous since the electrically conductive layer 206 is thin enough that it does not significantly affect the mechanical properties of the dielectric polymer film. The electrically conductive layer 206 may comprise thin metallic layers (e.g. aluminum foil, gold leaf, copper leaf, metallic hot or cold transfer films) that can also be used with a dielectric coating on one or both surfaces of the metallic layer or on the exposed surface of the sensing electrode 204. It is preferable that the dielectric coating of the first dielectric layer 218 is 0.1 μm<1 μm<3 μm<5 μm, <10 μm thick, without pinholes, and is not tacky or prone to surface blocking to avoid adhesion to the sensing electrode 204. The surface 214 of the sensing electrode 204 and/or the surface 222 of the first dielectric layer 218 or coating may be patterned or textured to reduce surface blocking.

Aluminum, gold, silver, and other metals can be used for the conductive coating of the electrically conductive layer 206. The electrically conductive layer 206 and the sensing electrode 204 can also be printed from conductive inks. The thickness of the printed features will need to be controlled to preserve the sensitivity of the proximity sensor 200.

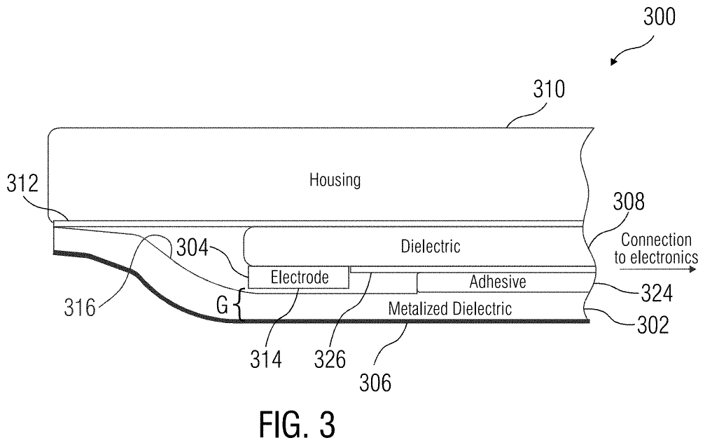

FIG. 3 shows an example proximity sensor 300 having a free floating foil construction with an adhesive layer 324 formed around a sensing electrode 304 or around an entire sensing electrode array, in accordance with at least one aspect of the present disclosure. In various aspects, the sensing electrode 304 may comprise multiple sensing elements or a sensing electrode array. The sensing electrode 304 may be adhered in some locations, particularly over a sensing electrode lead(s) 326 connecting the sensing electrode(s) 304 to the electronics to reduce/control parasitic electronic noise. As shown in FIG. 3, the adhesive layer 324 is positioned between a first dielectric layer 302 and the sensing electrode lead(s) 326.

In various aspects, the sensing electrode 304 may comprise multiple sensing elements or a sensing electrode array. In one aspect, the first dielectric layer 302 may be made of any suitable polymer and in one aspect, it is made of PET. A second dielectric layer 308 couples the sensing electrode 304 to a housing 310. In the illustrated example, the second dielectric layer 308 is mounted to the housing 310 via an adhesive 312. The second dielectric layer 308 also may be made of a suitable polymer and in the illustrated aspect is made of PET having a thickness of 150 μm for example. In one aspect, the second dielectric layer 308 has a thickness of up to 150 μm preferably up to 50 μm and more preferably from 1 μm to 25 μm. The housing 310 may be made of LDPE having a thickness in the range of 100 μm to 300 μm and preferably of 200 μm for example.

The thin first dielectric layer 302 separates the sensing electrode 304 from an electrically conductive layer 306, which may be ungrounded (not connected to electronics circuit), grounded, or connected to an antenna to extend the antenna range. A distance G separates the surface 314 of the sensing electrode 304 from the electrically conductive layer 306. Optimal results are obtained when the first dielectric layer 302 is thick enough to provide mechanical strength, elasticity and spring force to recover from a deformation, e.g. >1 μm>3 μm>5 μm>10 μm but thin enough to provide sensitivity, e.g. <25 μm<50 μm<150 μm. If the dielectric constant of the first dielectric layer 302 is sufficiently high, e.g. >5, >10, >50, >100, the first dielectric layer 302 can be thicker, up to 100-300 μm. In one aspect, the first dielectric layer 302 has a thickness of up to 150 μm preferably up to 50 μm and more preferably from 1 μm to 25 μm.

The first dielectric layer 302 may be a polymer film which has been metallized, e.g. through sputtering or other deposition/coating processes. The metallized film is particularly advantageous since the electrically conductive layer 306 is thin enough that it does not significantly affect the mechanical properties of the dielectric polymer film. The electrically conductive layer 306 may comprise thin metallic layers (e.g. aluminum foil, gold leaf, copper leaf, metallic hot or cold transfer films) that can also be used with a dielectric coating on one or both surfaces of the metallic layer or on the exposed surface of the sensing electrode 304. It is preferable that the dielectric coating of the first dielectric layer 302 is 0.1 μm<1 μm<3 μm<5 μm<10 μm thick, without pinholes, and is not tacky or prone to surface blocking to avoid adhesion to the sensing electrode 304. The surface 314 of the sensing electrode 304 and/or the surface 316 of the first dielectric layer 302 or coating may be patterned or textured to reduce surface blocking.

Aluminum, gold, silver, and other metals can be used for the conductive coating of the electrically conductive layer 306. The electrically conductive layer 306 and the sensing electrode 304 can also be printed from conductive inks. The thickness of the printed features will need to be controlled to preserve the sensitivity of the proximity sensor 300.

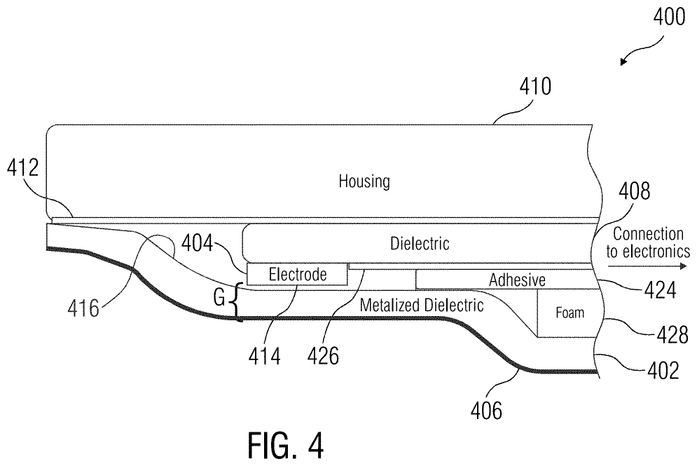

FIG. 4 shows an example proximity sensor 400 having a free floating foil construction with an additional layer of material 428 disposed over sensing electrode lead(s) 426, in accordance with at least one aspect of the present disclosure. The layer of material 428 may be dielectric, foam, or double-sided tape. The dielectric, foam or double-sided tape also can be used over the sensing electrode lead(s) 426 to reduce/control parasitic electronic noise. These extra layers of materials 428 need to be located sufficiently far from the sensing electrodes 404 so they do not increase the distance between the sensing elements of the sensing electrode 404 and the skin to the extent that the pulse-waveform can no longer be sensed with sufficient fidelity or signal-to-noise.

In various aspects, the sensing electrode 404 may comprise multiple sensing elements or a sensing electrode array. In one aspect, the first dielectric layer 402 may be made of any suitable polymer and in one aspect, it is made of PET. A second dielectric layer 408 couples the sensing electrode 404 to a housing 410. In the illustrated example, the second dielectric layer 408 is mounted to the housing 410 via an adhesive 412. The second dielectric layer 408 also may be made of a suitable polymer and in the illustrated aspect is made of PET having a thickness of 150 μm for example. In one aspect, the second dielectric layer 408 has a thickness of up to 150 μm preferably up to 50 μm and more preferably from 1 μm to 25 μm. The housing 410 may be made of LDPE having a thickness in the range of 100 μm to 300 μm and preferably of 200 μm, for example.

The thin first dielectric layer 402 separates the sensing electrode 404 from an electrically conductive layer 406, which may be ungrounded (not connected to electronics circuit), grounded, or connected to an antenna to extend the antenna range. A distance G separates the surface 414 of the sensing electrode 404 from the electrically conductive layer 406. Optimal results are obtained when the first dielectric layer 402 is thick enough to provide some mechanical strength, elasticity and spring force to recover from a deformation, e.g. >1 μm>3 μm>5 μm>10 μm but thin enough to provide sensitivity, e.g. <25 μm<50 μm<150 μm. If the dielectric constant of the first dielectric layer 402 is sufficiently high, e.g. >5, >10, >50, >100, the first dielectric layer 402 can be thicker, up to 100-300 μm. In one aspect, the first dielectric layer 402 has a thickness of up to 150 μm preferably up to 50 μm and more preferably from 1 μm to 25 μm.

The first dielectric layer 402 may be a polymer film which has been metallized, e.g. through sputtering or other deposition/coating processes. The metallized film is particularly advantageous since the electrically conductive layer 406 is thin enough that it does not significantly affect the mechanical properties of the dielectric polymer film. The electrically conductive layer 406 may comprise thin metallic layers (e.g. aluminum foil, gold leaf, copper leaf, metallic hot or cold transfer films) that can also be used with a dielectric coating on one or both surfaces of the metallic layer or on the exposed surface of the sensing electrode 404. It is preferable that the dielectric coating of the dielectric layer 402 is <1 μm<3 μm<5 μm<10 μm thick, without pinholes, and is not tacky or prone to surface blocking to avoid adhesion to the sensing electrode 404. The surface 414 of the sensing electrode 404 and/or the surface 416 of the first dielectric layer 402 or coating may be patterned or textured to reduce surface blocking.

Aluminum, gold, silver, and other metals can be used for the electrically conductive layer 406. The electrically conductive layer 406 and the sensing electrode 404 can be printed from conductive inks. The thickness of the printed features will need to be controlled to preserve the sensitivity of the proximity sensor 400.

2. Reference Sensors

Reference sensors can be fabricated by modulating the sensitivity of some of the elements of the sensor arrays. These sensors are not sensitive to changes in the pulse-waveform but may be able to sense changes due to large scale motions or environmental effects. The signal from the reference sensor(s) may be used to correct the signal from pulse-waveform sensor(s) to correct for baseline changes that may occur due to motion or environmental artifacts.

One method of creating reference sensors is to change the location or size of the active area of the electrodes of the reference sensors relative to the active area of the sensing electrodes 404. The reference might be smaller or located at a distance from the sensing elements to make it less probable that there is good positional overlap with a pulse point to pick up the pulse-waveform signal.

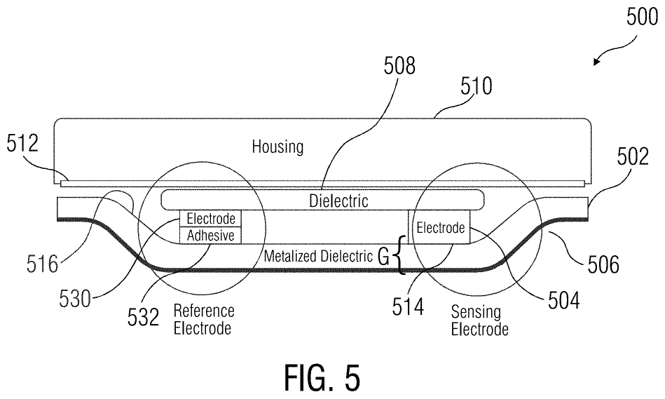

FIG. 5 shows an example proximity sensor 500 having a free floating foil construction with a reference electrode 530 and a sensing electrode 504, in accordance with at least one aspect of the present disclosure. The reference sensing electrode 530 may be created by attaching the first dielectric layer 502 with an electrically conductive layer 506 (e.g., foil) to an electrode with a fastener 532 such as adhesive, double-sided tape, and/or dielectric layers to prevent motion between the reference electrode 530 and the electrically conductive layer 506 to prevent the reference electrode 530 from responding to small changes in position due to motion of the skin which affect the motion of the electrically conductive layer 506 with respect to the sensing electrode 504. The reference electrode 530 can detect changes in capacitance due to motion of the entire sensor package or can detect changes in environmental conditions.

In all cases, the reference electrodes 530 need to be located sufficiently far from the sensing electrodes 504 such that they do not impact the sensitivity of the sensing electrodes 504 (through mechanical constraints) or increase the distance between the sensing electrode 504 elements and the skin to the extent that the pulse-waveform can no longer be sensed with sufficient fidelity or signal-to-noise. The impact of mechanical constraints can be mitigated by mechanically and/or positionally separating the reference electrode 530 elements from the sensing electrode 504 elements although care must be taken to position them in sufficiently similar positions that they will experience the same large-scale motions and environmental conditions.

The sensing electrode 504 will detect both small changes due to the pulse-waveform as well as larger motion and environmentally induced changes. In various aspects, the sensing electrode 504 and the reference electrode 530 each may comprise multiple sensing elements or a sensing electrode array. In one aspect, the first dielectric layer 502 may be made of any suitable polymer and in one aspect, it is made of PET. A second dielectric layer 508 couples the sensing electrode 504 and the reference electrode 530 to a housing 510. In the illustrated example, the second dielectric layer 508 is mounted to the housing 510 via an adhesive 512. The second dielectric layer 508 also may be made of a suitable polymer and in the illustrated aspect is made of PET having a thickness of 150 μm for example. In one aspect, the second dielectric layer 508 has a thickness of up to 150 μm preferably up to 50 μm and more preferably from 1 μm to 25 μm. The housing 510 may be made of LDPE having a thickness of 100 μm to 300 μm and preferably 200 μm, for example.

The thin first dielectric layer 502 separates the sensing electrode 504 and the reference electrode 530 from an electrically conductive layer 506, which may be ungrounded (not connected to electronics circuit), grounded, or connected to an antenna to extend the antenna range. A distance G separates the surface 514 of the sensing electrode 504 from the electrically conductive layer 506. Optimal results are obtained when the first dielectric layer 502 is thick enough to provide some mechanical strength, elasticity and spring force to recover from a deformation, e.g. >1 μm>3 μm>5 μm>10 μm but thin enough to provide sensitivity, e.g. <25 μm<50 μm<150 μm. If the dielectric constant of the first dielectric layer 502 is sufficiently high, e.g. >5, >10, >50, >100, the first dielectric layer 502 can be thicker, up to 100-300 μm. In one aspect, the first dielectric layer 502 has a thickness of up to 150 μm preferably up to 50 μm and more preferably from 1 μm to 25 μm.

The first dielectric layer 502 may be a polymer film which has been metallized, e.g. through sputtering or other deposition/coating processes. The metallized film is particularly advantageous since the metallic coating is thin enough that it does not significantly affect the mechanical properties of the dielectric polymer film. The electrically conductive layer 506 may comprise thin metallic layers (e.g. aluminum foil, gold leaf, copper leaf, metallic hot or cold transfer films) that can also be used with a dielectric coating on one or both surfaces of the metallic layer or on the exposed surface of the sensing electrode 504. It is preferable that the dielectric coating of the first dielectric layer 502 is <1 μm<3 μm<5 μm<10 μm thick, without pinholes, and is not tacky or prone to surface blocking to avoid adhesion to the sensing electrode 504. The surface 514 of the sensing electrode 504 and/or the surface 516 of the first dielectric layer 502 or coating may be patterned or textured to reduce surface blocking.

Aluminum, gold, silver, and other metals can be used for the electrically conductive layer 506. The electrically conductive layer 506 and the sensing electrode 504 can be printed from conductive inks. The thickness of the printed features will need to be controlled to preserve the sensitivity of the proximity sensor 500.

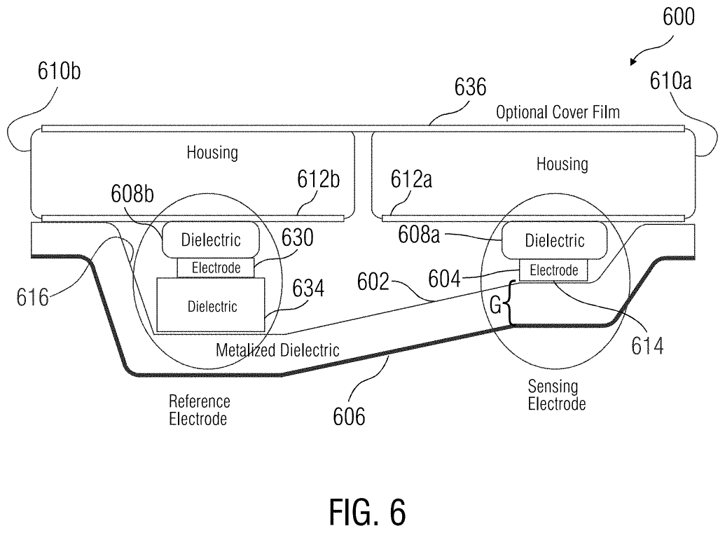

FIG. 6 shows an example proximity sensor 600 having a free floating foil construction with a layer of dielectric material 634 attached or coated onto a reference electrode 630 which is significantly thicker and/or has a significantly lower dielectric constant than the material used with the sensing electrode 604, in accordance with at least one aspect of the present disclosure. In various aspects, the layer of dielectric material 634 can be attached or coated onto some of the reference electrodes 630 in the electrode array which is significantly thicker and/or have a significantly lower dielectric constant than the materials used with other (sensing) electrodes 604 in the electrode array.

The sensitivity of single or multiple electrode elements can be modulated in an array of sensing electrodes 604. If multiple reference electrodes 630 are used, they can be tailored to have different sensitivities. If pairs of sensing/reference electrodes 604, 630 are used in a differential mode, either one or both sensing/reference electrode 604, 630 elements may be desensitized in the pair. It may be advantageous to configure the reference electrode 630 and the sensing electrode 604 to have similar overall signal levels, white noise and/or background signal levels for ease in subtracting one signal from the other.

In all cases, the reference electrodes 630 need to be located sufficiently far from the sensing electrodes 604 such that they do not impact the sensitivity of the sensing electrodes 604 (through mechanical constraints) or increase the distance between the sensing electrode 604 elements and the skin to the extent that the pulse-waveform can no longer be sensed with sufficient fidelity or signal-to-noise. The impact of mechanical constraints can be mitigated by mechanically and/or positionally separating the reference electrode 630 elements from the sensing electrode 604 elements although care must be taken to position them in sufficiently similar positions that they will experience the same large-scale motions and environmental conditions. In the case of mechanically isolated sensing elements, a cover film or sealant material 636 may be used to prevent the facile ingress of fluids.

The sensing electrode 604 will detect both small changes due to the pulse-waveform as well as larger motion and environmentally induced changes. In various aspects, the sensing electrode 604 and the reference electrode 630 each may comprise multiple sensing elements or a sensing electrode array. In one aspect, the first dielectric layer 602 may be made of any suitable polymer and in one aspect, it is made of PET. As shown in the figure, the sensing electrode 604 and the reference electrode 630 are mechanically isolated such that the sensing electrode 604 is coupled to a first housing 610a via a second dielectric layer 608a and the reference electrode 630 is coupled to a second housing 610b via a third dielectric layer 608b. Both the first and second housings 610a, 610b are covered by the cover film or sealant material 636. In the illustrated example, the second dielectric layer 608a is mounted to the first housing 610a via an adhesive 612a and the third dielectric layer 608b is mounted to the second housing 610b via an adhesive 612b. The second and third dielectric layers 608a, 608b also may be made of a suitable polymer and in the illustrated aspect is made of PET each having a thickness of 150 μm for example. In one aspect, the second and third dielectric layers 608a, 608b each have a thickness of up to 150 μm preferably up to 50 μm and more preferably from 1 μm to 25 μm. The housings 610a, 610b may be made of LDPE each having a thickness of 100 μm to 300 μm and preferably 200 μm, for example.

The thin first dielectric layer 602 separates the sensing electrode 604 and the reference electrode 630 from an electrically conductive layer 606, which may be ungrounded (not connected to electronics circuit), grounded, or connected to an antenna to extend the antenna range. A distance G separates the surface 614 of the sensing electrode 604 from the electrically conductive layer 606. Optimal results are obtained when the first dielectric layer 602 is thick enough to provide some mechanical strength, elasticity and spring force to recover from a deformation, e.g. >1 μm>3 μm>5 μm>10 μm but thin enough to provide sensitivity, e.g. <25 μm<50 μm<150 μm. If the dielectric constant of the first dielectric layer 602 is sufficiently high, e.g. >5, >10, >50, >100, the first dielectric layer 602 can be thicker, up to 100-300 μm. In one aspect, the first dielectric layer 602 has a thickness of up to 150 μm preferably up to 50 μm and more preferably from 1 μm to 25 μm.

The first dielectric layer 602 may be a polymer film which has been metallized, e.g. through sputtering or other deposition/coating processes. The metallized film is particularly advantageous since the electrically conductive layer 606 is thin enough that it does not significantly affect the mechanical properties of the dielectric polymer film. The electrically conductive layer 606 may comprise thin metallic layers (e.g. aluminum foil, gold leaf, copper leaf, metallic hot or cold transfer films) that can also be used with a dielectric coating on one or both surfaces of the metallic layer or on the exposed surface of the sensing electrode 604. It is preferable that the dielectric coating of the dielectric layer 602 is <1 μm<3 μm<5 μm<10 μm thick, without pinholes, and is not tacky or prone to surface blocking to avoid adhesion to the sensing electrode 604. The surface 614 of the sensing electrode 604 and/or the surface 616 of the first dielectric layer 602 or coating may be patterned or textured to reduce surface blocking.

Aluminum, gold, silver, and other metals can be used for the electrically conductive layer 606. The electrically conductive layer 606 and the sensing electrode 604 can be printed from conductive inks. The thickness of the printed features will need to be controlled to preserve the sensitivity of the proximity sensor 600.

3. Foam Layer for Improved Conformality

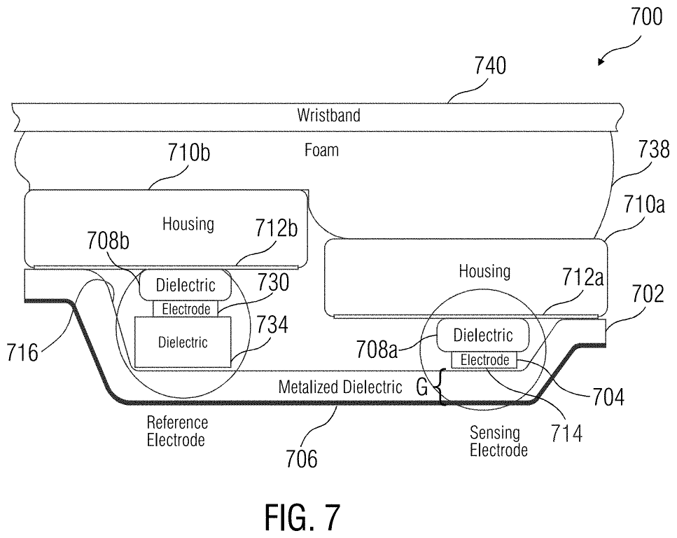

FIG. 7 shows an example proximity sensor 700 having a free floating foil construction with a foam layer 738 disposed between sensor elements (e.g., sensing electrode 704 and reference electrode 730) and a mounting structure 740 such as a wristband to provide conformity and ensure that both reference electrode 730 elements and sensing electrode 704 elements have similar contact to the skin, in accordance with at least one aspect of the present disclosure. The foam layer 738 between the sensing and reference electrode elements 704, 730 and a mounting structure 740 such as a wristband may be used to provide conformity and to ensure that both sensing and reference electrode elements 704, 730 have similar contact to the skin. The sensing and reference electrode elements 704, 730 optionally may be mechanically isolated as shown in the figure such that the sensing electrode 704 is coupled to a first housing 710a via a second dielectric layer 708a and the reference electrode 730 is coupled to a second housing 710b via a third dielectric layer 708b.

The band (or mounting structure 740) itself may be a foam such as EVA craft foam, cleanwipe foam, or medical foam (e.g. 3M 9776, 3M 1772, or Rosidal 77362). It is advantageous to use small-celled, open-celled foams that are compressible, breathable and/or stretchable yet provide sufficient mechanical integrity for use as a support material for the sensing and reference electrodes 704, 730 array and different fastening mechanisms such as hook-and-loop materials, eyelets and buckle clasps, cam buckles, and adhesives. Materials for the foam layer 738 with less mechanical integrity can be supported by lamination to another material such as loop fabric used for hook-and-loop fasteners. The foam layer 738 and/or additional laminated material may be perforated in some sections to increase stretchability and breathability. The use of one or more regions/layers of viscoelastic or dissipative materials which can partially or wholly absorb the impact of mechanical stimuli within different frequency ranges may be desired to assist in the mitigation of signal artifacts arising from different kinds of motion, vibrations, or environmental effects. These dissipative materials may be incorporated into the mounting structure 740 of the device or used as an accessory that partially isolates the patient's limb or body from the environment.

In all cases, the reference electrodes 730 need to be located sufficiently far from the sensing electrodes 704 such that they do not impact the sensitivity of the sensing electrodes 704 (through mechanical constraints) or increase the distance between the sensing electrode 704 elements and the skin to the extent that the pulse-waveform can no longer be sensed with sufficient fidelity or signal-to-noise. The impact of mechanical constraints can be mitigated by mechanically and/or positionally separating the reference electrode 730 elements from the sensing electrode 704 elements although care must be taken to position them in sufficiently similar positions that they will experience the same large-scale motions and environmental conditions. In the case of mechanically isolated sensing elements, a cover film or sealant material may be used to prevent the facile ingress of fluids.

The sensing electrode 704 will detect both small changes due to the pulse-waveform as well as larger motion and environmentally induced changes. In various aspects, the sensing electrode 704 and the reference electrode 730 each may comprise multiple sensing elements or a sensing electrode array. In one aspect, the first dielectric layer 702 may be made of any suitable polymer and in one aspect, it is made of PET. As shown in the figure, the sensing electrode 704 and the reference electrode 730 are mechanically isolated such that the sensing electrode 704 is coupled to a first housing 710a via a second dielectric layer 708a and the reference electrode 730 is coupled to a second housing 710b via a third dielectric layer 708b. In the illustrated example, the second dielectric layer 708a is mounted to the first housing 710a via an adhesive 712a and the third dielectric layer 708b is mounted to the second housing 710b via an adhesive 712b. The second and third dielectric layers 708a, 708b also may be made of a suitable polymer and in the illustrated aspect is made of PET each having a thickness of 150 μm for example. In one aspect, each of the second and third dielectric layers 708a, 708b have a thickness of up to 150 μm preferably up to 50 μm and more preferably from 1 μm to 25 μm. The housings 710a, 710b may be made of LDPE each having a thickness of 100 μm to 300 μm and preferably 200 μm, for example.

The thin first dielectric layer 702 separates the sensing electrode 704 and the reference electrode 730 from an electrically conductive layer 706, which may be ungrounded (not connected to electronics circuit), grounded, or connected to an antenna to extend the antenna range. A distance G separates the surface 714 of the sensing electrode 704 from the electrically conductive layer 706. Optimal results are obtained when the first dielectric layer 702 is thick enough to provide some mechanical strength, elasticity and spring force to recover from a deformation. e.g. >1 μm>3 μm>5 μm>10 μm but thin enough to provide sensitivity, e.g. <25 μm<50 μm<150 μm. If the dielectric constant of the first dielectric layer 702 is sufficiently high, e.g. >5, >10, >50, >100, the first dielectric layer 702 can be thicker, up to 100-300 μm. In one aspect, the first dielectric layer 702 has a thickness of up to 150 μm preferably up to 50 μm and more preferably from 1 μm to 25 μm.

The first dielectric layer 702 may be a polymer film which has been metallized, e.g. through sputtering or other deposition/coating processes. The metallized film is particularly advantageous since the electrically conductive layer 706 is thin enough that it does not significantly affect the mechanical properties of the dielectric polymer film. The electrically conductive layer 706 may comprise thin metallic layers (e.g. aluminum foil, gold leaf, copper leaf, metallic hot or cold transfer films) that can also be used with a dielectric coating on one or both surfaces of the metallic layer or on the exposed surface of the sensing electrode 704. It is preferable that the dielectric coating of the dielectric layer 702 is <1 μm<3 μm<5 μm<10 μm thick, without pinholes, and is not tacky or prone to surface blocking to avoid adhesion to the sensing electrode 704. The surface 714 of the sensing electrode 704 and/or the surface 716 of the first dielectric layer 702 or coating may be patterned or textured to reduce surface blocking.

Aluminum, gold, silver, and other metals can be used for the electrically conductive layer 706. The electrically conductive layer 706 and the sensing electrode 704 can be printed from conductive inks. The thickness of the printed features will need to be controlled to preserve the sensitivity of the proximity sensor 700.

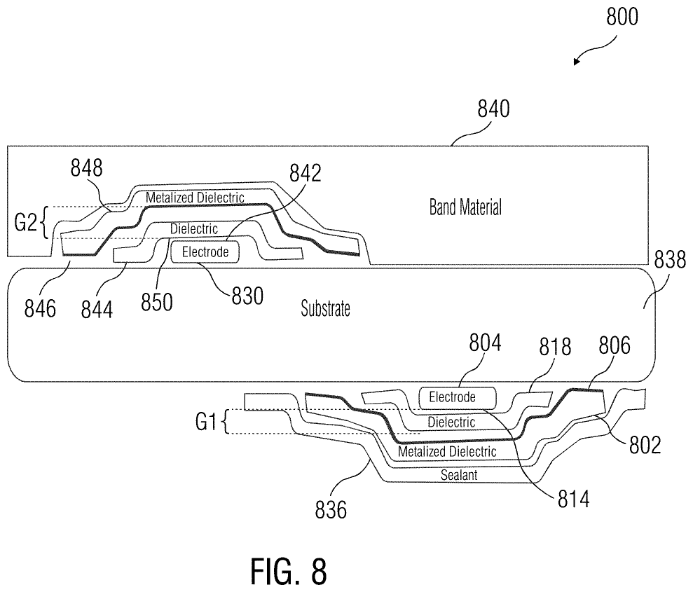

FIG. 8 shows an example proximity sensor 800 having a free floating foil construction with reference electrodes 830 located on an opposite side of a substrate layer 838 from sensing electrodes 804, in accordance with at least one aspect of the present disclosure. In some cases, it may be advantageous to place the reference electrodes 830 on the opposite side of the substrate layer 838 from the sensing electrodes 804. This enables the reference electrode 830 sensors to experience similar motions as the sensing electrode 804 sensors but with significantly lower exposure to the pulse signal. The substrate layer 838 may be made of any material including but not limited to foam, cloth, dielectric materials, conductive materials, leather, plastic, and combinations of these materials.

The sensing electrode 804 is located on one side of the substrate layer 838 and is separated from the electrically conductive layer 806 of a first dielectric layer 802 by a second dielectric layer 818. A sealant layer 836 (e.g. Tegaderm) covers the first dielectric layer 802 on the side that is opposite of the electrically conductive layer 806.

The reference electrode 830 is located on the other side of the substrate layer 838 and is separated from the electrically conductive layer 846 of a third dielectric layer 848 by a fourth dielectric layer 844. The reference electrode 830 sensor stack is embedded within a mounting structure 840.

The band (or mounting structure 840) itself may be a foam such as EVA craft foam, cleanwipe foam, or medical foam (e.g. 3M 9776, 3M 1772, or Rosidal 77362). It is advantageous to use small-celled, open-celled foams that are compressible, breathable and/or stretchable yet provide sufficient mechanical integrity for use as a support material for the sensing and reference electrodes 804, 830 array and different fastening mechanisms such as hook-and-loop materials, eyelets and buckle clasps, cam buckles, and adhesives. Materials for the substrate layer 838 with less mechanical integrity can be supported by lamination to another material such as loop fabric used for hook-and-loop fasteners. The substrate layer 838 and/or additional laminated material may be perforated in some sections to increase stretchability and breathability. The use of one or more regions/layers of viscoelastic or dissipative materials which can partially or wholly absorb the impact of mechanical stimuli within different frequency ranges may be desired to assist in the mitigation of signal artifacts arising from different kinds of motion, vibrations, or environmental effects. These dissipative materials may be incorporated into the mounting structure 840 of the device or used as an accessory that partially isolates the patient's limb or body from the environment.

In all cases, the reference electrodes 830 need to be located sufficiently far from the sensing electrodes 804 such that they do not impact the sensitivity of the sensing electrodes 804 (through mechanical constraints) or increase the distance between the sensing electrode 804 elements and the skin to the extent that the pulse-waveform can no longer be sensed with sufficient fidelity or signal-to-noise. The impact of mechanical constraints can be mitigated by mechanically and/or positionally separating the reference electrode 830 elements from the sensing electrode 804 elements although care must be taken to position them in sufficiently similar positions that they will experience the same large-scale motions and environmental conditions. In the case of mechanically isolated sensing elements, a cover film or sealant material 836 may be used to prevent the facile ingress of fluids.

The sensing electrode 804 will detect both small changes due to the pulse-waveform as well as larger motion and environmentally induced changes. In various aspects, the sensing electrode 804 and the reference electrode 830 each may comprise multiple sensing elements or a sensing electrode array. In one aspect, the first and third dielectric layers 802, 848 may be made of any suitable polymer and in one aspect, it is made of PET. The second and fourth dielectric layers 818, 844 also may be made of a suitable polymer and in the illustrated aspect is made of PET each having a thickness of 150 μm for example.

The thin first and third dielectric layers 802,848 separates the sensing electrode 804 and the reference electrode 830 from respective electrically conductive layers 806, 846, which may be ungrounded (not connected to electronics circuit), grounded, or connected to an antenna to extend the antenna range. A distance G1 separates the surface 814 of the sensing electrode 804 from the electrically conductive layer 806. A distance G2 separates the surface 842 of the reference electrode 830 from the electrically conductive layer 846. Optimal results are obtained when the first and third dielectric layers 802, 848 are thick enough to provide some mechanical strength, elasticity and spring force to recover from a deformation, e.g. >1 μm>3 μm>5 μm>10 μm but thin enough to provide sensitivity, e.g. <25 μm<50 μm<150 μm. If the dielectric constant of the first and third dielectric layers 802, 848 is sufficiently high, e.g. >5, >10, >50, >100, the first and third dielectric layers 802, 848 can be thicker, up to 100-300 μm. In one aspect, the first and third dielectric layers 802, 846 each may have a thickness of up to 150 μm preferably up to 50 μm and more preferably from 1 μm to 25 μm.

The first and third dielectric layers 802, 848 may be made of a polymer film which has been metallized, e.g. through sputtering or other deposition/coating processes. The metallized film is particularly advantageous since the electrically conductive layers 806, 846 is thin enough that it does not significantly affect the mechanical properties of the dielectric polymer film. The electrically conductive layers 806, 846 may comprise thin metallic layers (e.g. aluminum foil, gold leaf, copper leaf, metallic hot or cold transfer films) that can also be used with a dielectric coating on one or both surfaces of the metallic layer or on the exposed surface of the sensing or reference electrodes 804, 830. It is preferable that the dielectric coating of the dielectric layer 702 is <1 μm<3 μm<5 μm<10 μm thick, without pinholes, and is not tacky or prone to surface blocking to avoid adhesion to the sensing or reference electrodes 804, 830. The surfaces 814, 842 of the sensing and reference electrodes 804, 830 and/or the surfaces 816, 850 of the second and fourth dielectric layers 818, 844 or coating may be patterned or textured to reduce surface blocking.

Aluminum, gold, silver, and other metals can be used for the conductive coating of the electrically conductive layers 806, 846. The conductive coating of the electrically conductive layers 806, 846 and the sensing or reference electrodes 804, 830 can be printed from conductive inks. The thickness of the printed features will need to be controlled to preserve the sensitivity of the proximity sensor 800.

4. Materials Selection

Sensor attachment: A number of materials can be used for a band, patch, or other method to fasten the sensor arrays of the proximity sensors 100, 200, 300, 400, 500, 600, 700, 800, 1000, 1100, 1200, 1300 described in FIGS. 1-8, 12, 13, 15, 16 to the skin. The material may be flexible, thin, slightly elastic or stretchable, and optionally somewhat breathable (semi-permeable or semi-occlusive) and water-resistant, for comfort and ease of use. Some preferred materials include self-adhesive bandage material (e.g. 3M Coban), medical tape (e.g. 3M Microfoam Surgical tape) kinesiology tape (e.g. RockTape or TheraBand), EVA foam, cleanwipe foam (e.g. Foamtec Cleanwipes), foam such as used for infant ID bands (e.g. PDC Precision neonatal bands or GBS EasyID bands), medical foams (e.g. 3M 9776), silicone, polyurethane, styrene copolymers, acrylic copolymers, fluorinated copolymers, polyolefins, ethylene vinyl acetate, neoprene, PVC, and similar thermoplastic and thermoset elastomers. The materials may be solid material or foams with or without texture and/or cut-outs or perforations for stretchability or breathability, or woven or non-woven fabrics, or a combination (e.g. laminates or adhered/connected/sewn sections) of these (e.g. Goretex fabric, NexCare bandages, Tegaderm dressings, Glad Press'n Seal wrap). To fasten the band around a body part or onto the skin, these materials can be self-adhesive or have significant surface tack or may use sections with hook-and-loop material (e.g. Velcro), adhesive (including silicone, acrylate, polyurethane), or watchband-type buckles or clasps. Materials with surface tack (e.g. silicone or Fabrifoam), materials backed with adhesives such as kinesiology tape (e.g. RockTape or Kinesio tape), or nanostructured dry-adhesive surfaces (e.g. Setex) can be used to minimize motion of the sensor array with respect to the skin. Commercial watchbands of metal, leather, silicone, polyurethane, and other polymeric materials have also been used for longer term use. Latex-free and nickel-free materials are preferred to avoid allergic reactions or skin irritation.

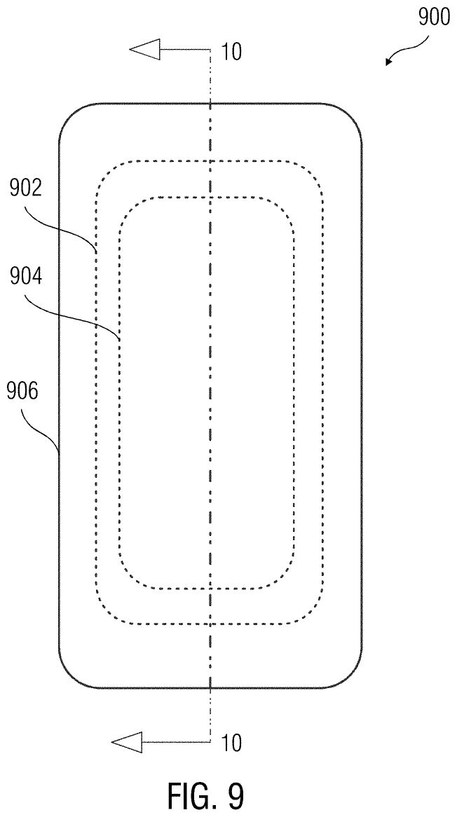

FIG. 9 shows one view of an example attachment structure 900 for a proximity sensor having a free floating foil construction, such as the proximity sensors 100, 200, 300, 400, 500, 600, 700, 800, 1000, 1100, 1200, 1300 described in FIGS. 1-8, 12, 13, 15, 16, where the attachment structure includes a number of materials used for a band, patch, or other method to fasten the sensor array to the skin, in accordance with at least one aspect of the present disclosure.

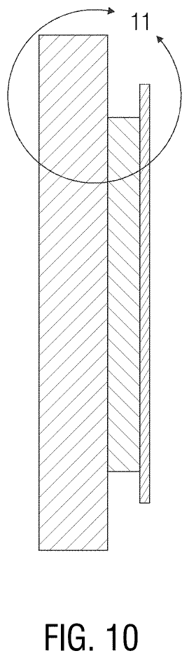

FIG. 10 shows a section view of the example attachment structure 900 shown in FIG. 9, taken along section line 10-10, in accordance with at least one aspect of the present disclosure.

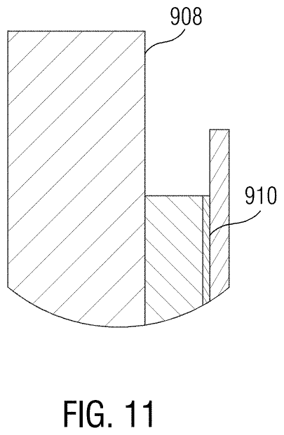

FIG. 11 shows a detail view of the section view of the example attachment structure 900 shown in FIG. 10, taken along line 11, in accordance with at least one aspect of the present disclosure.

With reference now to FIGS. 9-11, the sensor assembly may comprise material layers that can be laminated together, for example as a die cut sticker, prior to assembly of the band to simplify the manufacturing process. It may be advantageous to use island lamination of patches of different materials to create this sticker where adhesive is applied only around the perimeter of the patches, enabling some or all of the materials at the center of the patches to move independently. The attachment structure 900 includes three materials 902-906, an adhesive side 908, and a metallized side 910.

In one example, a first material 902 may be a PET film <5 μm thick, a second material 904 may be a PET film ˜12 μm thick metalized with aluminum on the metallized side 910, and a third material 906 may be a polyurethane film with adhesive on one adhesive side 908 ˜25 μm total thickness. A patch of the second material 904 may be adhered to a patch of the third material 906. A larger patch of the first material 902 may then be laminated to the composite of the second and third materials 904, 906 such that it is adhered around the perimeter of the patch of the second material 904 as shown in FIGS. 9-11.

In another example, an adhesive may be printed on the adhesive side 908 as a pattern defining the perimeter of the patch area onto the second material 904. The first material 902 is then laminated onto the second material 904. The composite of the first and second materials 902, 904 is then die-cut, laser cut, or otherwise singulated, so it can then be island laminated onto the third material 906.

An example assembly process using may comprise: (1) make a slit in a band, e.g. fabricated by laminated medical foam and loop material or a pre-made identification band such as those from PDC or GBS, and insert the sensor flex circuit for the sensor array through this slit. The sensor flex circuit is optionally adhered to the band; (2) use a sticker to hold and seal the sensor flex circuit in place; and (3) connect the electronics to the flex circuit.

Electronics or sensor packaging for the proximity sensors 100, 200, 300, 400, 500, 600, 700, 800, 1000, 1100, 1200, 1300 described in FIGS. 1-8, 12, 13, 15, 16 may comprise films with adhesive or blocking (self-adhering) surfaces (e.g. polyolefin packing tapes, Tegaderm dressings, Glad Press'n Seal wrap, silicone or polyurethane film) that also can be used as disposable packaging for reusable electronics or for the sensor elements. These materials can be wrapped around the electronics, battery/power supply and/or sensor array and attached to a band or patch with adhesive, double-sided tape or hook-and-loop material, snaps or other low-cost, low-profile attachment method.

In another aspect, molded cases or clamshell housings may be employed which can be reversibly sealed with press-fit closures or snap-fits. Suitable materials for these include silicone, polyurethane, polyolefins, acrylates, polyesters, PETG, EVA, and copolymers and blends of these materials. Vacuum forming or thermoforming, injection molding, rotational casting, blow-molding, or reaction injection molding can be used to fabricate the housings.

Re-engageable contacts may be provided between the electronics module and the sensor/electrode leads. In one aspect, printed conductive elastomer bumps may be provided for elastically compressible re-engageable contacts.

In one aspect, some or all of the electronics and/or the battery/power supply for the proximity sensors 100, 200, 300, 400, 500, 600, 700, 800, 1000, 1100, 1200, 1300 described in FIGS. 1-8, 12, 13, 15, 16 are packaged in a module or “pebble” separate from the sensor array. The electronics module may be encapsulated or sealed. For establishing electrical contact between the sensor array and the electronics module and/or battery/power supply, it can be advantageous to use a structure to electrically connect the components that is easy to use and low cost. One such method comprises printing, stenciling, or molding elastomeric connection points onto the leads of the sensor array. The materials could be an elastomeric conductive polymer formulation or a carbon or metal filled polymeric composite, e.g. conductive ink used for polymer solder bumps. It can be advantageous to use thixotropic materials that may cure quickly to create high profile structures, preferably >0.25 mm, >0.5 mm, >1 mm tall, 3-d or aerosol jet printers can also used to create high profile structures. Materials that are somewhat compliant, elastomeric and do not exhibit significant compression set are preferred to enable a degree of deformation upon contacting the conductive pad(s) on the electronics module with the conductive bump(s) on the sensor electrode lead(s) with a small amount of compression force. One such material is ThreeBond TB3333E silver filled silicone which can be dispensed with an air-actuated adhesive or solder paste dispenser.

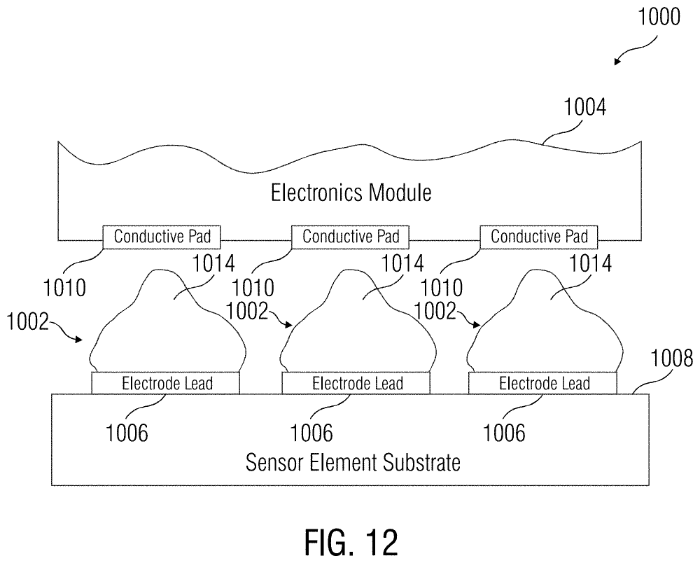

FIG. 12 shows an example proximity sensor 1000 comprising re-engageable contacts 1002 between an electronics module 1004 and sensor/electrode leads 1006 having printed conductive elastomer conductive bumps 1014 to form elastically compressible re-engageable contacts 1002, in accordance with at least one aspect of the present disclosure.

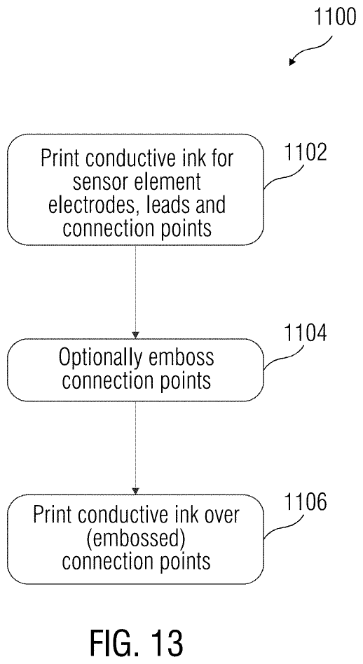

The proximity sensor 1000 is representative of any one of the proximity sensors 100, 200, 300, 400, 500, 600, 700, 800, 1000, 1100, 1200, 1300 described in FIGS. 1-8, 12, 13, 15, 16. The re-engageable contacts 1002 electrically connect the sensor/electrode leads 1006 on a sensor element substrate 1008 that supports the proximity sensor 1000 to conductive pads 1010 located on the electronics module 1004. In one aspect, the elastomer conductive bumps 1014 of the re-engageable contacts 1002 are formed of thixotropic elastomeric conductive ink according to a process discuss below in connection with FIG. 13.

FIG. 13 shows an example method 1100 of printing conductive elastomer bumps 1002 shown in FIGS. 12, 14, and 15 for elastically compressible re-engageable contacts, in accordance with at least one aspect of the present disclosure. With reference to FIGS. 12 and 13, the method 1100 comprises printing 1102 conductive ink 1014 for the sensor element electrodes, leads 1006, and connection points on a substrate 1008. Optionally, the method 1100 comprises embossing 1104 the connection points. The method 1100 comprises printing 1106 conductive ink 1014 over the embossed connection points. For a reusable electronics module 1004, it is desirable that the conductive pads 1010 are sealed against the surface of the electronic module 1004 for ease of cleaning after each use.

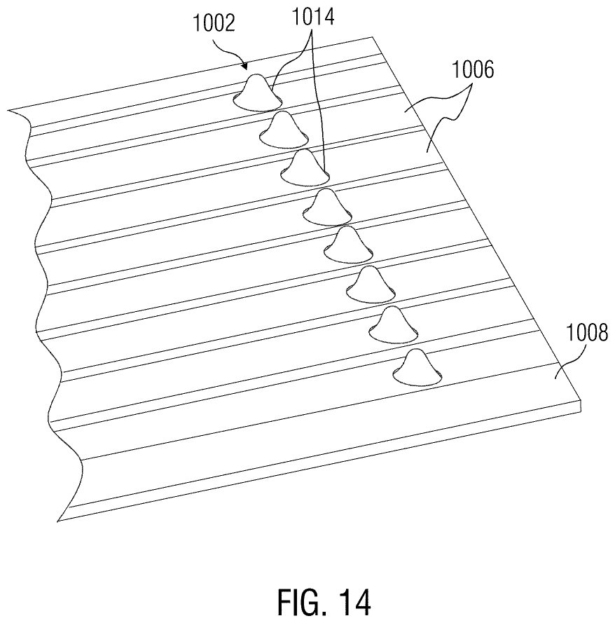

With reference also to FIG. 12, FIG. 14 shows an example of conductive elastomer bumps 1002 printed on electrode leads 1006 to be pressed against an electronics module 1004, in accordance with at least one aspect of the present disclosure.

With reference to FIGS. 12-14, features such as bosses in a tray or clamshell can be used to hold the electronic module 1004 or a printed circuit board in place, providing sufficient pressure to deform the elastomeric conductive bumps 1002 with the conductive pads 1010 on the electronic module 1004 and achieve an electrical contact between the sensor element substrate 1008 and the electronic module 1004.

Still with reference to FIGS. 12-14, in one aspect, the conductive elastomer bumps 1002 can alternatively be printed of conductive ink 1014 on the electronics module 1004 and pressed against the electrode leads 1006. This may be less appropriate for applications where reusable electronics need to be mated to the electrode leads 1006 multiple times since the conductive elastomer bumps 1002 may not be robust enough for multiple uses.

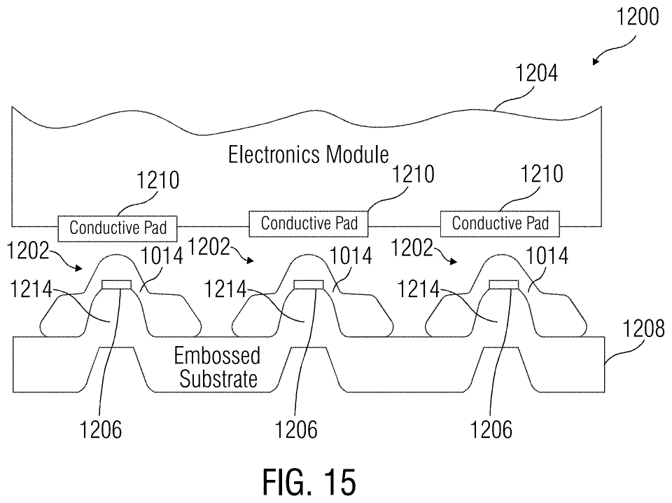

FIG. 15 shows an example proximity sensor 1200 comprising conductive elastomer bumps 1202 fabricated by embossing structures 1214 into a sensor element substrate 1208 that supports the sensing/reference electrodes of the proximity sensor 1200, in accordance with at least one aspect of the present disclosure. The proximity sensor 1200 is representative of the proximity sensors 100, 200, 300, 400, 500, 600, 700, 800, 1000, 1100, 1200, 1300 described in FIGS. 1-8, 12, 13, 15, 16. The conductive bumps 1202 can also be fabricated by embossing structures 1214 into the sensor element substrate 1208 that support the electrodes as described in connection with FIG. 13.

With reference to FIGS. 13 and 15, the electrode leads 1206 are disposed on the embossed structures 1214 and are then overprinted with conductive ink 1014. The electrode leads 1206 can optionally be formed on the sensor element substrate 1208 before embossing 1104 the connection points. It can be advantageous to emboss 1104 the regions around the areas of the electrode leads 1206 (i.e. emboss a flat plateau around the electrode lead 1206) to minimize the loss of conductivity in the electrode leads 1206. In the event that it is necessary to emboss 1104 the conductive material that forms the electrode leads 1206, it is advantageous to minimize the slope of the embossed structures 1212 to minimize the loss of conductivity in the electrode leads 1206. For example, while it is possible to provide a steep wall along the sides of the electrode lead 1206, it would be best to provide a gentle slope to the deformed region of the embossed structures 1212 when embossing 1104 across the electrode lead 1206. The conductive elastomer bumps 1202 electrically contact conductive pads 1210 disposed on an electronics module 1204.

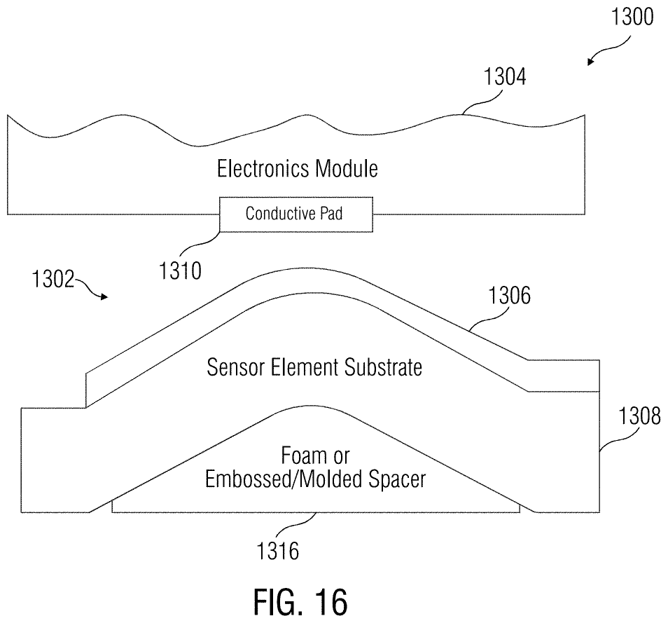

FIG. 16 shows a partial view of an example proximity sensor 1300 comprising conductive features 1302 fabricated by mechanically deforming electrical leads 1306, in accordance with at least one aspect of the present disclosure. The mechanically deformed electrode leads 1306 are disposed over the sensor element substrate 1308. The proximity sensor 1300 is representative of any one of the proximity sensors 100, 200, 300, 400, 500, 600, 700, 800, 1000, 1100, 1200, 1300 described in FIGS. 1-8, 12, 13, 15, 16. Another method of providing low-cost connections between the electronics module 1304 and the sensor array of the proximity sensor 1300 includes mechanically deforming the electrical leads 1306 by backing them with a compliant spacer 1316 such as a molded elastomer part or a piece of foam that is optionally shaped to optimize the curvature of the deformed electrode lead 1306 to control the contact area between the electrode lead 1306 and a conductive pad 1310 on the electronics module 1304. In addition, providing a support frame such as the compliant spacer 1316 around the contact points formed by the conductive features 1302 also may improve help control the contact area.

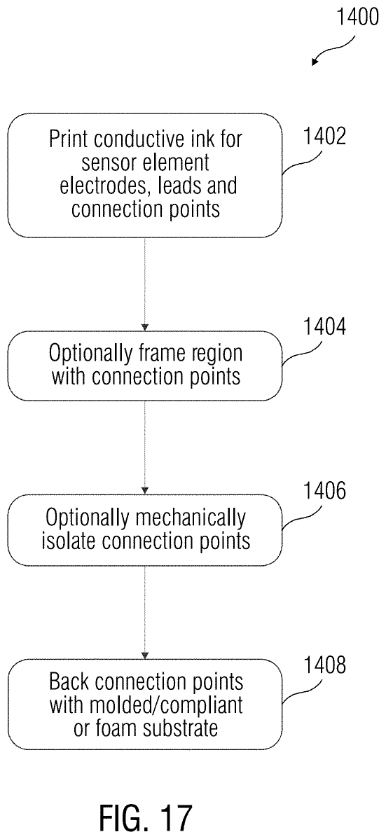

FIG. 17 shows an example method 1400 of forming connections between an electronics module 1304 and a sensor array by mechanically deforming electrical leads 1306 as shown in FIG. 16, in accordance with at least one aspect of the present disclosure. With reference now to FIGS. 16 and 17, in one aspect, the method 1400 comprises printing 1402 conductive ink for the sensor element electrodes, leads 1306, and connection points. Optionally, the method 1400 comprises framing 1404 a region with connection points. Also optionally, the method 1400 comprises mechanically isolating 1406 the connections points. The method 1400 further comprises backing 1408 the connection points with molded/compliant or foam substrate such as the compliant spacer 1316.

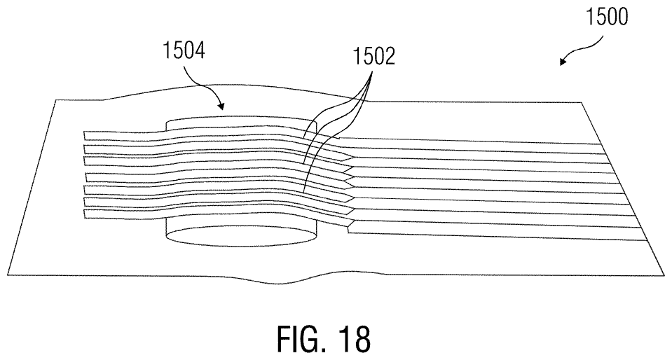

FIG. 18 shows an example connector 1500 formed by the method 1400 described in FIG. 17 having mechanically isolated individual electrode leads 1502 in an array 1504 of electrode leads with improved compliance, in accordance with at least one aspect of the present disclosure. This configuration may improve the compliance of individual electrode leads 1502 in an array of electrode leads to mechanically isolate them.

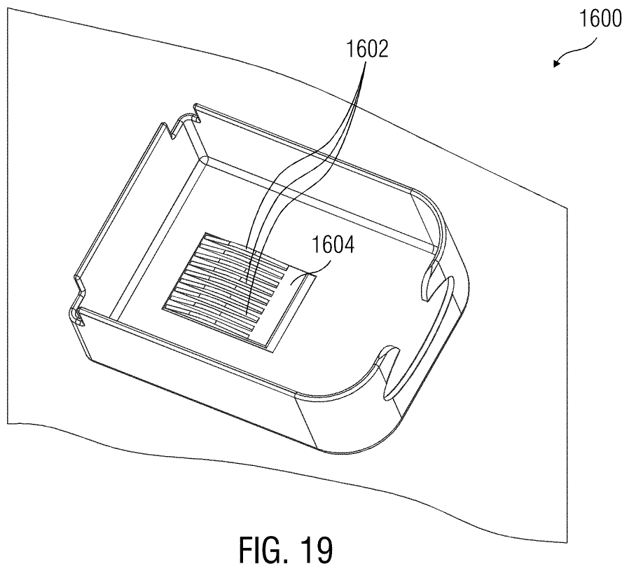

FIG. 19 shows an example connector 1600 formed by the method described in FIG. 17 having mechanically rigid spring fingers 1602 supported and deformed with foam or other spacer material 1604, in accordance with at least one aspect of the present disclosure. Additional mechanically rigid spring fingers 1602 can be used instead of more compliant electrodes that are optionally supported and/or deformed with a foam or other spacer material 1604. The connectors 1500, 1600 can be insert-molded or press fit or otherwise incorporated into a bezel or receiver to hold the electronics in place.

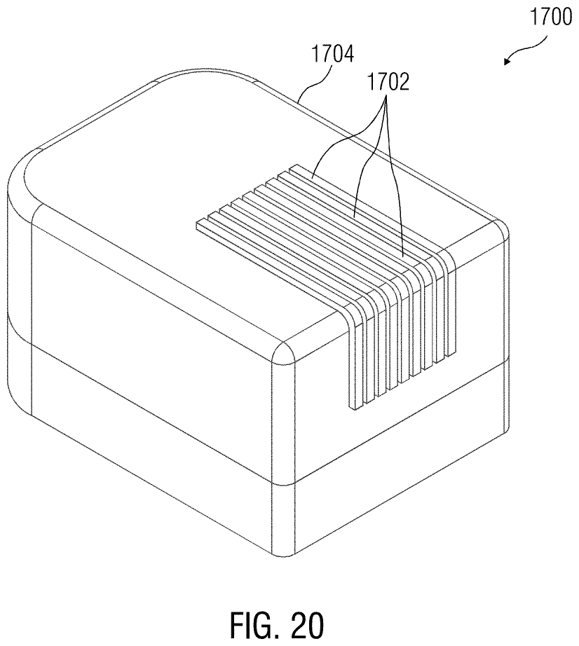

FIG. 20 shows an example electronic module 1700 with mating contacts 1702 formed on a housing 1704 of the electronics module 1702 used in conjunction with the connector 1600 shown in FIG. 19, in accordance with at least one aspect of the present disclosure. In an alternative aspect, spring fingers 1602 may be employed on the electronics module 1702 which press against the electrode leads.

### System Configuration

Mounting structures 740, 840 shown in FIGS. 7 and 8, respectively, may be implemented in the form of bands, patches, or other suitable structures. Bands can be adjustably sized to fit radial, brachial, tibial, dorsal, and/or femoral pulse points. Patches can be applied to other pulse points where bands may be difficult to apply such as carotid, temporal, on the hand or finger and behind the ear. Band and patch materials may be somewhat stretchable using materials such as self-adhesive bandage material (e.g. 3M Coban), EVA, silicone, polyurethane, styrenic copolymer, olefinic copolymer, stretchable hook-and-loop materials (e.g. 3M Velstrap), foam, dressing materials (e.g. 3M Tegaderm) and fabric. Sensors can also be incorporated into bands fabricated from less stretchable materials such as leather, vinyl, metal mesh, nylon mesh, fabric, hook-and-loop straps (e.g. 3M Velcro) and other typical watch band materials. The bands can be fastened with hook-and-loop closures, buckles, snaps, magnets and other fastening methods often used with watch bands.

Sensor elements comprising sensing electrodes 114, 214, 314, 414, 514, 614, 714, 814 as shown in FIGS. 1-8, and/or reference electrodes 530, 630, 730, 830 as shown in FIGS. 5-8, can be positioned on the mounting structures 740, 840, such as bands and/or patches, such that they can be located in the vicinity of a pulse point. Arrays of sensor elements may be used to provide a level of positional tolerance for ease of use. Sensor elements may be arranged in a fan out to improve positional tolerance with respect to the pulse point. It may be advantageous to distribute sensor elements along the length of the band in order to pick up multiple pulse points simultaneously such as dorsal and tibial locations or radial and ulnar locations. The sensor elements can be positioned individually or in pairs. They can be operated singly or in differential mode, either subtracting one from the other for baseline correction or as the two legs of an LC tank circuit (e.g. in the manner of TI FDC2214) for greater sensitivity and noise exclusion.

The sensor elements can have dimensions with an aspect ratio >1 and the longer axis can be oriented parallel to the direction of the artery for better coupling and higher signal or perpendicular to the artery for greater positional tolerance. Lengths between 5 and 30 mm and widths of 0.25 mm to 2 mm may be advantageous to balance positional tolerance and signal quality. Different elements or pairs of elements may have different orientations. It may be advantageous that the distance between pairs of sensor elements is minimized to the limit of the fabrication process; distances between elements of less than 0.5 mm may be advantageous for improving signal quality in differential mode. Sensing and/or reference electrodes for the sensor elements may be fanned out at the connection to the electronics for ease of alignment.

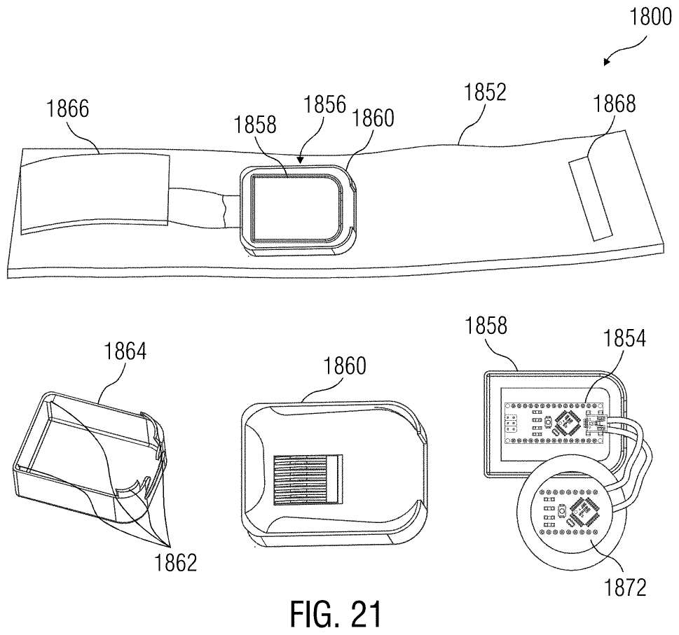

FIG. 21 shows an example sensor band 1800 comprising a band 1852 and an electronics module 1856, in accordance with at least one aspect of the present disclosure. The band 1852 is configured for adults adjustably sized to fit radial, brachial, tibial, dorsal, and/or femoral pulse points. The band 1852 may be adjustably secured to an adult using a low profile hook 1866 fastener secured to a low profile loop fabric 1868. The electronics module 1856 (“pebble”) is sealed or partially sealed and comprises electronic circuits 1854 electrically coupled to a battery 1872 and one or more proximity sensor(s) located on the opposite side of the band 1852. In one aspect, the electronic circuits 1854 and the battery 1872 may be reusable and the proximity sensor(s) and the band 1852 are disposable. The reusable electronic circuits 1854 and disposable proximity sensor(s) 1872 snap fit into a tray 1858 and cover 1864 housing. The electronics module 1856 is received into a shell 1870 that is fastened to the band 1852 through known fastening methods. The reusable electronic circuits 1854 and battery 1872 snap fit into the tray 1858 and cover 1864. The surface design of the electronics module 1856 (“pebble”) may be substantially smooth to facilitate cleaning with antibiotic wipes. The proximity sensor(s) on band 1852 may be configured as any one or more of the proximity sensors 100, 200, 300, 400, 500, 600, 700, 800, 1000, 1100, 1200, 1300 described in FIGS. 1-8, 12, 13, 15, 16.

Encapsulation could be effected through overmolding, insert molding, potting, or casting. Materials for the tray 1808 include silicone, polyurethane, styrenic copolymer, olefinic copolymer, ABS, PET, polyolefins, nylon, polycarbonate, PETG. The multi-part case of the electronics module 1856 is assembled around the reusable electronic circuits 1854 and disposable proximity sensor(s) 1872 and fastened to the cover 1864 through fasteners 1862 such as snap-fits, adhesive, heat-welds, or other known fastening methods, can be used.

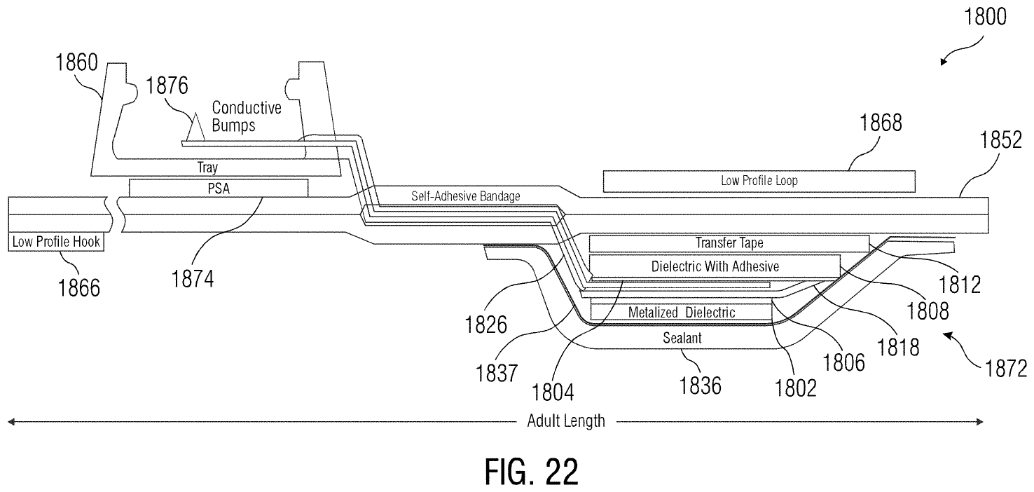

Conductive leads or vias may be incorporated into the shell 1860 (e.g. through insert molding) to make connections to the sensor electrodes. Alternatively the sensor electrode leads may be fed into the shell 1860 through a slot in the side wall or through the bottom of the shell 1860 with alignment facilitated by molded features in the shell 1860. Magnets may be used to assist with alignment and to secure the connection between the shell 1860 and the electronics module 1856 (“pebble”). A schematic section view of the proximity sensor 1800 comprising a band 1852 configured for adults is described below in connection with FIG. 22.

FIG. 22 shows a schematic section view of the sensor band 1800 comprising the band 1852 for adults and the electronics module 1856 shown in FIG. 21, in accordance with at least one aspect of the present disclosure. As described in connection with FIG. 21, the band 1852 is sized and configured for adults. With reference now to both FIGS. 21 and 22, the sensor band 1800 comprises a disposable proximity sensor 1872 fixed to the band 1852 and covered by a sealant layer 1836. The sealant layer 1836 comprises an adhesive 1837 to attach to the band 1852 and the first dielectric layer 1802. The band 1852 may be formed of a light weight cohesive elastic which provides controlled, consistent compression and conforms to all body contours. The laminate of nonwoven material and elastic fibers placed lengthwise, provide excellent elasticity. The band 1952 material adheres to itself without the use of pins, clips or tape. In one aspect, the band 1852 may be made of a material known in the industry as Coban.

The disposable proximity sensor 1872 comprises a first dielectric layer 1802 comprising an electrically conductive layer 1806 coupled to a sensing electrode 1804. The sensing electrode 1804 is electrically coupled to a conductive bump 1876 located in a shell 1860 fixed to the band 1852 by a pressure sensitive adhesive 1874 (PSA) and configured to receive the electronics module 1856. The sensing electrode 1804 is electrically coupled to the conductive bump 1876 by a connector shown in FIG. 22 as a flat flexible cable 1826 (FFC) that extends from the disposable proximity sensor 1872 to the shell 1860 for electrically coupling to the electronics module 1856. A second dielectric layer 1806 is disposed between the sensing electrode 1804 and the first dielectric layer 1802. A third dielectric layer 1808 with adhesive is attached to the FFC 1826 on one side and is attached to the band 1852 on the other side by way of another adhesive layer 1812. Accordingly, the sensing electrode 1804 is electrically coupled to the electronics module 1856.

In one aspect, the sealant layer 1836 may be a 25 μm polyurethane layer with adhesive layer 1837 known in the industry as Tegaderm. In one aspect, the first dielectric layer 1802 may be a 12 μm PET layer with an aluminum (AL) electrically conductive layer 1806. In one aspect, the second dielectric layer 1806 may be a 2.5 μm PET. In one aspect, the third dielectric layer 1808 may be a 25 μm polypropylene with acrylic adhesive. The adhesive layer 1812 is a 12 μm ultrathin acrylic transfer tape. In one aspect, the length of the band 1852 is ˜9 inches, which is sized for an adult wearer. Notwithstanding the example dimensions provided in this section, the dimensions of the various dielectric layers of the proximity sensor 1872 may be selected in accordance with the dimensions described herein in connection with the proximity sensors 100, 200, 300, 400, 500, 600, 700, 800, 1000, 1100, 1200, 1300 described in FIGS. 1-8, 12, 13, 15, 16, for example.

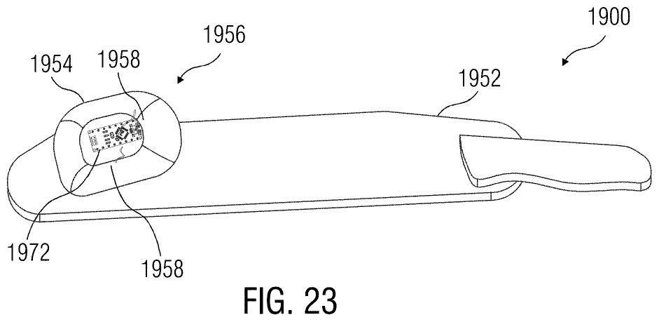

FIG. 23 shows an example sensor band 1900 comprising a band 1952 and an electronics module 1956, in accordance with at least one aspect of the present disclosure. The band 1952 is configured for infants adjustably sized to fit radial, brachial, tibial, dorsal, and/or femoral pulse points, in accordance with at least one aspect of the present disclosure. The electronics module 1956 may not be fully sealed and may be wrapped in a disposable film or encased in a clamshell housing. The clamshell housing can be molded from a soft elastomeric material such as silicone, polyurethane, styrenic copolymer, olefinic copolymer, polyolefin or EVA, or a more rigid material such as PETG, PET, nylon, polycarbonate or ABS. The clamshell housing can be closed temporarily with a friction fit and/or bosses or closed permanently with adhesive or heat-staking. The clamshell housing also can be attached permanently to the band 1952 with adhesive or heat-welding or temporarily with hook-and-loop fastener material. The proximity sensor(s) located on band 1952 on the side opposite from the electronics module (not shown in the figure) may be configured as any one or more of the proximity sensors 100, 200, 300, 400, 500, 600, 700, 800, 1000, 1100, 1200, 1300 described in FIGS. 1-8, 12, 13, 15, 16.

The electronics module 1956 comprises electronic circuits 1954 and a battery 1972. The electrode leads 1958 are connected to proximity sensors on the opposite side of band 1952 and may be fed into the clamshell housing of the electronics module 1956 through a slot defined in a side wall, in a gap between the lid and bottom of the clamshell housing, or through the bottom of the clamshell housing with alignment facilitated by molded features in the clamshell housing. Bosses may be used to secure the electronic circuits 1954 within the clamshell housing and provide sufficient spring force to maintain electrical contact between electrode leads 1958 and the electronic circuits 1954. Magnets may be used to assist with alignment and to secure the connection between the electrodes in the clamshell housing and the electronic circuits.

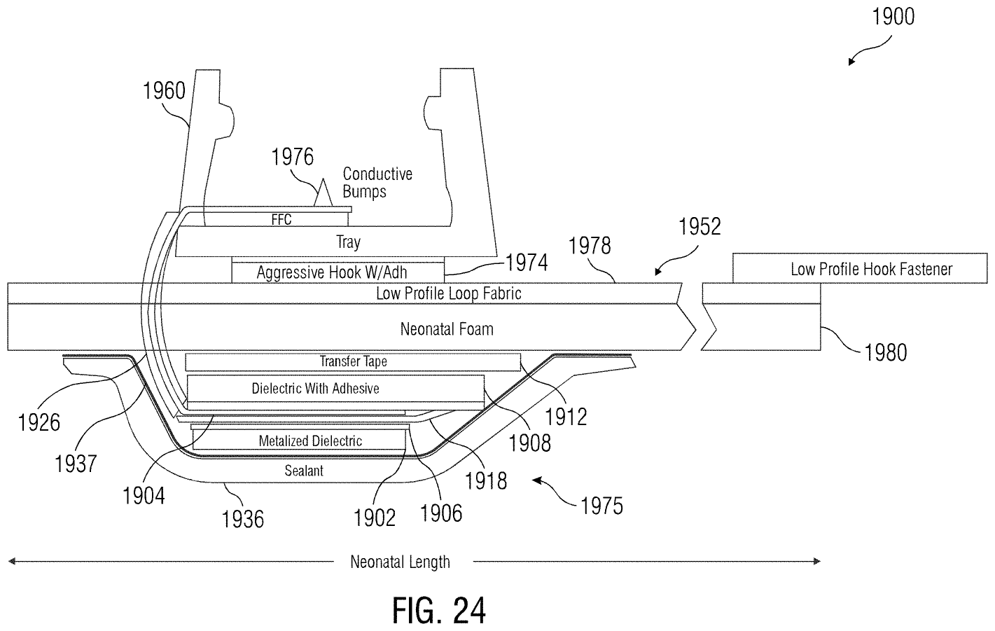

Conductive material can be incorporated into the band 1952, tray/clamshell housing, and/or proximity sensor architecture, that is electrically connected to the ground plane or antennae on the electronic circuits 1954 to improve radio performance. A schematic section view of the sensor band 1900 comprising a band 1952 configured for infants is described below in connection with FIG. 24.

FIG. 24 shows a schematic section view of the sensor band 1900 comprising the band 1952 for infants and the electronics module 1956 shown in FIG. 23, in accordance with at least one aspect of the present disclosure. As described in connection with FIG. 23, the band 1952 is sized and configured for infants. With reference now to both FIGS. 23 and 24, the sensor band 1900 comprises a disposable proximity sensor 1975 fixed to the band 1952 and covered by a sealant layer 1936. The sealant layer 1936 comprises an adhesive 1937 to attach to the band 1952 and the first dielectric layer 1902. The band 1952 may be formed of a low profile loop fabric 1978 disposed over neonatal foam 1980.

The disposable proximity sensor 1975 comprises a first dielectric layer 1902 comprising an electrically conductive layer 1906 coupled to a sensing electrode 1904. The sensing electrode 1904 is electrically coupled to a conductive bump 1976 located in a shell 1960 fixed to the band 1952 by an aggressive hook material 1974 with adhesive and configured to receive the electronics module 1956. The sensing electrode 1904 is electrically coupled to the conductive bump 1976 by a connector shown in FIG. 23 as a flat flexible cable 1926 (FFC) that extends from the disposable proximity sensor 1975 to the shell 1960 for electrically coupling to the electronics module 1956. A second dielectric layer 1906 is disposed between the sensing electrode 1904 and the first dielectric layer 1902. A third dielectric layer 1908 with adhesive is attached to the FFC 1926 on one side and is attached to the neonatal foam 1980 of the band 1952 on the other side by way of another adhesive layer 1912. Accordingly, the sensing electrode 1904 is electrically coupled to the electronics module 1956.

In one aspect, the sealant layer 1936 may be a 25 μm polyurethane layer with an adhesive layer 1937 known in the industry as Tegaderm. In one aspect, the first dielectric layer 1902 may be a 12 μm PET layer with an aluminum (AL) electrically conductive layer 1906. In one aspect, the second dielectric layer 1906 may be a 2.5 μm PET. In one aspect, the third dielectric layer 1908 may be a 25 μm polypropylene with acrylic adhesive. The adhesive layer 1912 is a 12 μm ultrathin acrylic transfer tape. In one aspect, the length of the band 1952 is ˜6-8 inches, which is sized for an infant wearer. Notwithstanding the example dimensions provided in this section, the dimensions of the various dielectric layers of the proximity sensor 1975 may be selected in accordance with the dimensions described herein in connection with the proximity sensors 100, 200, 300, 400, 500, 600, 700, 800, 1000, 1100, 1200, 1300 described in FIGS. 1-8, 12, 13, 15, 16, for example.

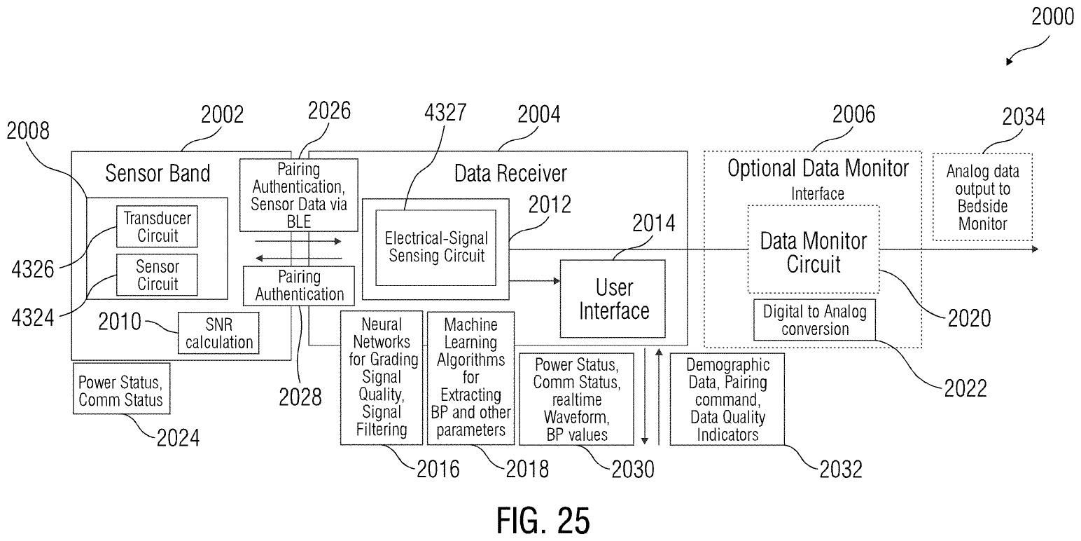

FIG. 25 illustrates a system 2000 that employs the sensor bands and proximity sensors described herein in connection with FIGS. 1-24, in accordance with at least one aspect of the present disclosure. Generally, the system 2000 comprises circuitry that processes a signal received by the proximity sensing circuits and converts the signal to an analog signal that can be read directly by a bedside monitor, emulating the transducer of an arterial line.

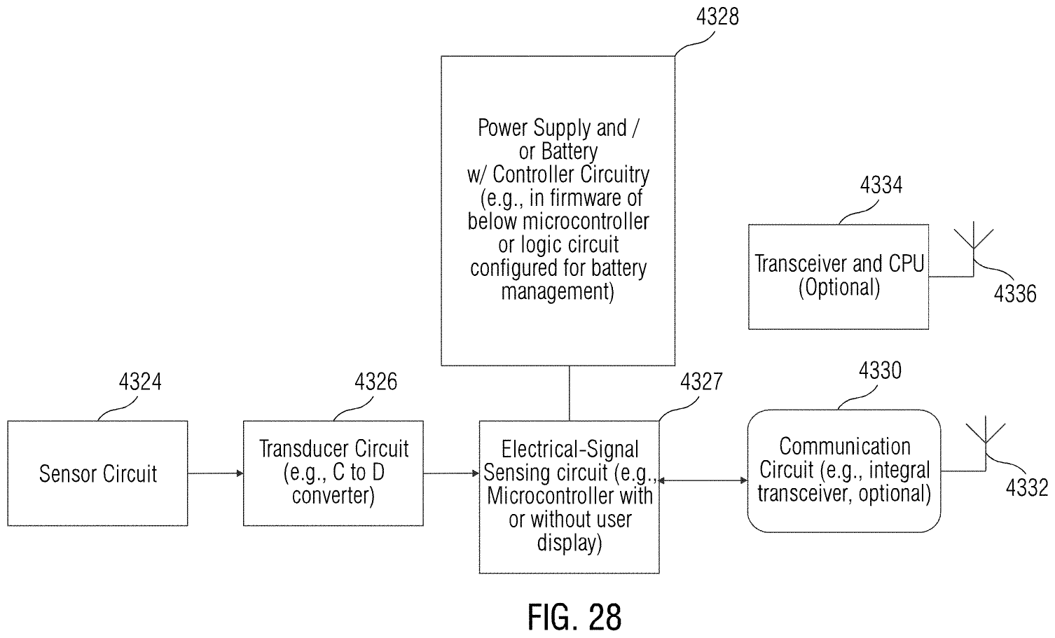

The system 2000 comprises a sensor band 2002 in communication with a data receiver 2004 optionally in communication with a data monitor interface 2006. The sensor band 2000 is representative of the sensor bands 1800, 1900 described in connection with FIGS. 21-24. The sensor band 2002 comprises a sensor circuit module 2008 (e.g. printed circuit board assembly (PCBA) and firmware) to detect signals from a patient's body using any one of the proximity sensors 100, 200, 300, 400, 500, 600, 700, 800, 1000, 1100, 1200, 1300 described in FIGS. 1-8, 12, 13, 15, 16. In one aspect, the signal detected by the proximity sensor is a pulse-waveform that represents one or more physiological parameters including, for example, blood pressure. In one aspect, the sensor band circuit module 2008 comprises a sensor circuit 4324 and a transducer circuit 4326 as described in FIG. 28 hereinbelow. The sensor band circuit module 2008 provides 2026 pairing, authentication, and sensor data via a wireless communication standard such as Bluetooth Low Energy (BLE), for example, to a receiver circuit module 2012 portion of the data receiver 2004. The receiver circuit module 2012 provides 2028 pairing authentication to the sensor circuit module 2008. The sensor circuit module 2008 also provides 2024 power and communication status and calculates 2010 signal-to-noise ratio.

The data receiver 2004 comprises a circuit module 2012 that includes hardware and software to process the signals received from the sensor band 2002 circuit module 2008. In one aspect, the receiver circuit module 2012 comprise an electrical-signal sensing circuit 4327 and a communication circuit 4330 as described in FIG. 28 hereinbelow. The circuit module 2012 also provides 2028 pairing authentication to the sensor band 2002 circuit module 2008. The receiver circuit module 2012 executes 2016 neural network algorithms for grading signal quality and provides signal filtering. The receiver circuit module 2012 also executes machine learning algorithms 2018 for extracting physiological parameters such as blood pressure (BP) and other physiological parameters from the sensor data received from the sensor band 2002 circuit module 2008. The receiver circuit module 2012 is coupled to a user interface 2014 to provide 2030 power status, communication status, real-time waveforms, and physiological parameters such as BP. The user interface 2014 receives 2032 demographic data, pairing commands, and data quality indicators for use by the receiver circuit module 2012 in the neural network and machine learning algorithms.

The optional data monitor interface 2006 comprises a data monitor circuit module 2020 configured to receive information from the receiver circuit module 2012. The data monitor circuit module 2020 converts 2022 the digital data input received from the receiver circuit module 2012 to an analog data output 2034 suitable for a bedside monitor.

Data can be transferred wirelessly from the proximity sensor and the sensor band 2002 to the data receiver 2004, which may be implements as a mobile device through standard protocols, e.g. Bluetooth. The data may be cached and sent in bursts or with variable packet size, e.g. DLE, to improve transmission efficiency. The data also can be stored locally either in the sensor band electronics module 2008 or on the data receiver 2004 (e.g. mobile device) to be post-processed at a later time.

The data may be preprocessed by the sensor band electronics module 2008 or on the data receiver 2004 (e.g. mobile device) with Fourier analysis and/or bandpass filters. SNR may be used to grade the quality of the data to choose sensor data streams to transmit only the best channels to a receiving device. Accelerometer or reference sensor data at the sensor band 2002 can be used to identify and/or quantify specific activities and can also be used to identify noisy data (e.g. motion artifacts) that should be flagged or excluded so it is not used for further analysis.

The data may be processed to extract relevant information, e.g. signal quality, hemodynamic parameters such as blood pressure, pulse height, heart rate, BP and heart rate (HR) variability, trends, and event probabilities, locally on the sensor band electronics module 2008, on the data receiver 2004 (e.g. mobile device/base station), or in the cloud.

A method for secure out of band pairing of Bluetooth radios includes using the data channel of an inductive charge system to pass keys from the transmit module 2008 to the receive module 2012, it is possible to securely pair the transmit and receive devices 2002, 2004. This eliminates the security concerns of in-band pairing, eliminates complexity of manual pairing, automates the pairing process, and requires no additional hardware, only the software routines to manage the pairing process. This technique can be applied to other non-contact charge strategies such as radio frequency power delivery, or contact based charging means such as contact pins.

A method for inductive charge energy shielding includes inductive charge systems rely on coupled electromagnetic transmit-receive systems which can expose the electronics on the data receiver 2002 to electromagnetic energy. This energy can cause eddy currents in the receiving printed circuit board assembly (PCBA) electronics module 2012, which can in turn generate heat in the PCBAs. One method of combating this issue is to shield the receiving PCBA from the electromagnetic energy by installing a thin ferrite sheet between the receive coil and the PCBA behind it.

Additional proximity sensor circuits and related sensing methods are disclosed in international application publication no. WO 2017/172978 A1, which is incorporated herein by reference in its entirety. A portion of referenced international application publication no. WO 2017/172978 A1 is reproduced hereinbelow for convenience.

Aspects of various aspects are directed to proximity sensors 100, 200, 300, 400, 500, 600, 700, 800, 1000, 1100, 1200, 1300 described in FIGS. 1-8, 12, 13, 15, 16 and related sensing methods for sensing hemodynamic changes (or pulse-waveforms) of a user.

In certain example aspects, aspects of the present disclosure involve one or more sensor circuits configured and arranged to sense hemodynamic changes (or pulse-waveforms) of a user with the sensor circuit configured in a manner to monitor the physiologic changes of the user by using a single electrode placed near/onto a surface to be measured. These and other aspects employ the sensor circuit configured to sense the hemodynamic changes consistent with one more of the below-described aspects and/or mechanisms.

More specific example aspects are directed to an apparatus having at least one sensor circuit, the sensor circuit including an electrode, and an electrical-signal sensing circuit. The apparatus can be used to monitor one or more of the hemodynamic parameters in a non-invasive manner and in real-time. For example, the electrical-signal sensing circuit can sense pulse-wave events, while the sensor circuit is placed near or onto skin, by monitoring capacitance changes. The capacitance changes carried by the electrode are responsive to pressure and/or electric field modulations attributable to the pulse-wave events or to the changes in pressure or blood flow in the blood vessels (e.g., hemodynamics). The electrode can be used to determine capacitance changes between the electrode and the skin of the user. The sensor circuit including the electrode can be arranged with a transducer circuit, which is used to provide an electrical signal to the electrical-signal sensing circuit indicative of the changes in capacitance and/or pressure. Due to pulse-wave events, the distance between the skin of the user and the electrode can change and/or the electric field distribution around the blood vessels can change, resulting in a relative change in capacitance as measured using the sensor circuit. The changes in capacitance over time can be processed by the electrical-signal sensing circuit and used to generate and/or determine a pulse-waveform. In various aspects, the pulse-waveform is correlated with various hemodynamic parameters. As specific examples, the pulse-waveform can be processed to determine a heart rate, blood pressure, arterial stiffness, and/or blood volume.

The electrode portion of the proximity sensors 100, 200, 300, 400, 500, 600, 700, 800, 1000, 1100, 1200, 1300 described in FIGS. 1-8, 12, 13, 15, 16 can be in contact with the skin of the user and/or in proximity. In some aspects, the electrode is constrained onto (whether in contact or not) the user using a mechanical constraint (e.g., a wristband, an elastically compliant band, or an article of clothing) and/or an adhesive. The electrode can be located near a blood vessel, preferably near a palpable pulse point such as but not limited to the radial, brachial, carotid, tibial, and temporal pulse points.

In other specific aspects, the apparatus includes a plurality of electrodes. For example, the apparatus can include a plurality of sensor circuits and each sensor circuit includes one of the plurality of electrodes. The plurality of electrodes can be arranged as part of a transducer circuit, which is used to provide electrical signals (e.g., a digital) to the electrical-signal sensing circuit indicative of the changes in capacitance that are responsive to modulations in distance between the skin of the user and the electrode, pressure and/or electric field and attributable to hemodynamic or pulse-wave events. In various related aspects, the plurality of sensor circuits are mechanically separated and/or arranged in an array (e.g., a sensor array). Each of the sensor circuits can be constructed differently, such as having different geometries, dielectric layers, locations, sensitivities, among other constructions as further described herein.

Various aspects are directed to a method of using the above-described apparatus. The method can include placing at least one electrode of an apparatus near or onto the skin of the user and sensing pulse-wave events. The pulse-wave events can be sensed while the at least one electrode is placed near or onto the skin of a user, using an electrical-signal sensing circuit of the apparatus, by monitoring capacitance changes that are responsive to pressure and/or electric field modulations attributable to hemodynamic or pulse-wave events. The pulse-wave events can be used to generate a pulse-waveform and/or to determine various hemodynamic parameters. For example, the method can include determining diastolic blood pressure, systolic blood pressure, arterial stiffness, and/or blood volume using the pulse-wave events.

A specific method can include use of flexible or bendable substrate of a wearable apparatus to secure the transducer circuit having at least one sensor circuit. The substrate supports and at least partially enclosing the transducer circuit and the electrical-signal sensing circuit. The substrate further conforms to a portion of a user including blood vessels and locates the at least one electrode sufficiently close to the user's skin for electrically sensing hemodynamic or pulse-wave events via the capacitance changes, the changes in capacitance being responsive to pressure and/or electric field modulations attributable to hemodynamic or pulse-wave events. A transducer circuit converts the changes in capacitance into the electrical signals. The method further includes sensing the hemodynamic or pulse-wave events in response to the electrical signals from the transducer circuit via the electrical-signal sensing circuit and using a communication circuit, within or outside the wearable apparatus, to respond to the electrical-signal sensing circuit by sending hemodynamic-monitoring data to an external circuit.

Other aspects are directed to an apparatus for use as part of a wearable device characterized by a flexible or bendable substrate configured and arranged to support and at least partially enclose a transducer circuit and an electrical-signal sensing circuit and to conform to a portion of a user including blood vessels for hemodynamic monitoring. The apparatus includes the transducer circuit having at least one sensor circuit, the sensor circuit including an electrode, the electrical-signal sensing circuit, and a communication circuit, as previously described above.

Aspects of the present disclosure are believed to be applicable to a variety of different types of apparatuses including, and methods involving use of, a user-worn sensor circuit configured and arranged to sense pulse wave events aspects, conditions and/or attributes of the user. In certain implementations, aspects of the present disclosure have been shown to be beneficial when used in the context of a wrist-located or wrist-worn strap but it will be appreciated that the instant disclosure is not necessarily so limited. Various aspects may be appreciated through the following discussion of non-limiting examples which use exemplary contexts.

Various aspects of the present disclosure are directed toward an apparatus including at least one sensor circuit having an electrode and an electrical-signal sensing circuit. The apparatus can be used to monitor one or more hemodynamic parameters and pulse-wave events in a non-invasive manner and in real-time. Surprisingly, it has been discovered that a common floating ground and single electrode that does not need to touch a users skin can be used for measuring pulse-wave events. In various aspects, pulse-wave events can be monitored in a hands-free manner and without interference from environmental noise (e.g., human voices and other background noise, electrical interference and ambient light). The electrode (or array of electrodes) can consume relatively low amounts of power (e.g., between 5 microwatts and 3 milliwatts, although aspects are not so limited). In some specific aspects, the power consumption can be further reduced by only saving data after a trigger event (e.g., heart rate above a threshold, a particular heart event occurs such as an event indicative of a problem) and/or transmitting saved data in burst transmissions. The electrical-signal sensing circuit can sense pulse-wave events, while the at least one electrode is placed near or onto skin, by monitoring pressure differentials attributable to the pulse-wave events or capacitance changes attributable to the pulse-wave events.

The electrode can be used to determine capacitance changes between the electrode and the skin of the user. Due to pulse-wave events, the distance between the skin of the user and the electrode can change, resulting in a relative change in capacitance and/or signal amplitude and quality as measured by the transducer circuit and the electrical-signal sensing circuit. The changes in capacitance over time can be processed by the electrical-signal sensing circuit and used to generate and/or determine a pulse-waveform. In various aspects, the pulse-waveform is correlated with various hemodynamic parameters. As specific examples, the pulse-waveform can be processed to determine a heart rate, blood pressure, arterial stiffness, and/or blood volume.

The electrode can be in contact with the skin of the user and/or in proximity. In some examples, the electrode can be sufficiently close to the user's skin for electrically sensing the hemodynamic or pulse-wave events via the capacitance changes carried by the electrode (or plurality of electrodes). In such examples, “sufficiently close” corresponds to a proximal distance, relative to the portion including the blood vessels, in a range from the furthest distance being 1 millimeter (mm) away from the skin and the nearest distance being zero, or in contact with the skin. In some aspects, the sensor circuit (e.g., the electrode) is constrained onto (whether in contact or not) the user using a mechanical constraint (e.g., flexible or bendable substrate, such as a wristband, sock, glove, sleeve, or other piece of wearable device or clothing) and/or an adhesive.

The changes in capacitance carried by the electrode and respective sensor circuit are responsive to pressure and/or electric field modulations attributable to hemodynamic or pulse-wave events. More specifically, the sensor circuit and electrode can capture (or sense) the capacitance changes through proximity sensing of the skin of the user (as opposed to physically deforming the device as a traditional capacitance sensor), and thereby act as or is a proximity sensor. The proximity sensing and/or capacitance changes are responsive to modulating distances between the skin of the user and the sensor circuit and/or modulating fringe field lines.

In other specific aspects, the apparatus includes a plurality of electrodes. The plurality of electrodes can be arranged as part of a transducer circuit, which is used to provide a signal to the electrical-signal sensing circuit indicative of the changes in capacitance and/or pressure. For example, the transducer circuit can have a plurality of sensor circuits and each of the sensor circuits includes one of the plurality of electrodes. The electrical-signal sensing circuit can be arranged with the transducer circuit to monitor pressure differentials less than 1 kPa, such as in a range between 0.3 kilopascal (kPa) to 1 kPa. The different electrodes can have different geometries, sensitivities and/or be at different locations. The transducer circuit can convert changes in capacitance into electrical signals (e.g., digital signals). As described herein, the transducer circuit and the electrical-signal sensing circuit can be supported by and at least partially enclosed by the substrate.

Certain aspects of the present disclosure are directed toward a method of using an apparatus, as previously described. The method can include placing at least one electrode of an apparatus near or onto the skin of the user and sensing pulse-wave events. The pulse-wave events can be sensed while the at least one electrode is placed near or onto the skin of a user, using an electrical-signal sensing circuit of the apparatus, by monitoring pressure differentials attributable to the pulse-wave events and/or monitoring capacitance changes (or relative capacitance changes) attributable to the pulse-wave events. The pulse-wave events can be used to generate a pulse-waveform and/or to determine various physiological and/or hemodynamic parameters. For example, the method can include determining diastolic blood pressure, systolic blood pressure, arterial stiffness, and/or blood volume using the pulse-wave events.

Somewhat surprisingly, pulse-wave events can be monitored using one or more electrodes placed on or near an arterial pulse point. For example, responsive to a pulse-wave event, each electrode can provide a signal indicative of the pulse-wave event. The electrode (or plurality of electrodes) is connected to circuitry, such as a transducer circuit. More specifically, each electrode (e.g., an electrical conductor) is connected to a respective sensor circuit which is used to measure or detect the signal indicative of the pulse-wave event (e.g., capacitance value and/or changes in capacitance) from the electrode and provides the signal to the transducer circuit. The transducer circuit than converts the signal indicative of the pulse-wave event to an electrical signal, which is provided to the electrical-signal sensing circuit. A pulse-wave event includes or refers to hemodynamic responses and/or attributes caused by and/or indicative of heart beats (e.g., contraction of heart muscles) (e.g., heart beats or sounds, pulsing of blood, etc.). The electrical-signal sensing circuit (and/or the transducer circuit) can include a commercially available or custom-designed circuit for capacitive touch screens and can be in wireless or wired communication with a central processing circuit (CPU). Further, the transducer circuit and/or the sensor circuit can have a floating ground. The signals measured using the electrode can be due to small pressure differences and/or surface displacements of the skin that modulate the fringe field at the electrode and resulting in measurable capacitance changes. The electrode(s) can be attached to the skin of a user (or other animal or being) using an adhesive (e.g., tape) or mechanically using a strap, such as a watchband, bracelet or wristband.

In specific aspects, the electrode(s) is encapsulated with a dielectric layer (e.g., an encapsulant). When a plurality of electrodes are used, the dielectric layer on each of the plurality of electrodes can have different structural characteristics to modulate signal sensitivity of each electrode. Example characteristics can include thickness of the dielectric layer, composition of the dielectric material used, structures, and resistivity values, among other characteristics. Each of the plurality of electrodes can be associated with different characteristics based on at least one of electrode geometry and dielectric layers used with the electrode. The different electrodes can be used to output signals, responsive to monitored pulse-wave events. Signals from different electrodes can be used in a differential mode to eliminate signals that may be common to the electrodes, such as temperature changes and user motion (e.g., noise), and to enhance signals or pulse-wave events which may be measured by the more sensitive electrodes, such as pulse-waveform pressure differentials. In related specific aspects, one or more of electrodes of the plurality of electrodes can be electrically shielded or isolated from one another. Further, spacers can be used to control or set the distance between at least one of the sensor circuits and/or electrodes and the skin of the user.

The signals provided by the electrode(s) can be used to determine various hemodynamic parameters. For example, responsive to a pulse-wave event, one or more signals indicative of capacitance changes are provided to the electrical-signal sensing circuit. As previously described, the capacitance changes, which are carried by at least one electrode, are responsive to pressure and/or electric field modulations attributable to hemodynamic or pulse-wave events. The electrical-signal sensing circuit uses the one or more signals to determine a heart rate, diastolic blood pressure, systolic blood pressure, arterial stiffness, and other hemodynamic parameters. The signals can be processed using one or more bandpass filters or other signal processing techniques. For example, the signal can be filtered either digitally or through a circuit design used to minimize artifacts due to factors such as pressure changes or motion due to breathing, arm motions, and external vibrations. Alternatively, characteristics of the artifacts can be isolated and quantified to extract parameters such as respiration rates and movement of the user. In one aspect, respiration rates can be measured from motion of the body and in other aspects from the pulse waveform.

These surprising findings can be particularly useful for monitoring blood pressure or other hemodynamic parameters in a non-invasive and/or continuous manner. In specific implementations, an apparatus can be used for providing sensitivity for pressure differentials and/or capacitance changes caused by pulse-wave events. Further, the apparatus and/or portions of the apparatus (e.g., the electrode) can be fabricated more easily than capacitive sensors as they have fewer design elements and material, making the end apparatus more robust.

In related-specific implementations, the apparatus includes or is a portion of a portable/wearable device and/or apparatus that can continuously monitor heart rate and other hemodynamic effects such diastolic blood pressure, systolic blood pressure, and arterial stiffness. As an example, a smart bandage can be applied over an arterial pulse point and can transmit data in real time to a receiver. Another example includes a smart watch band that provides a real-time read-out, stores and/or transmits the data. Other implementations are directed to small surface displacements or pressure differentials that can modulate the fringe fields at the electrode.

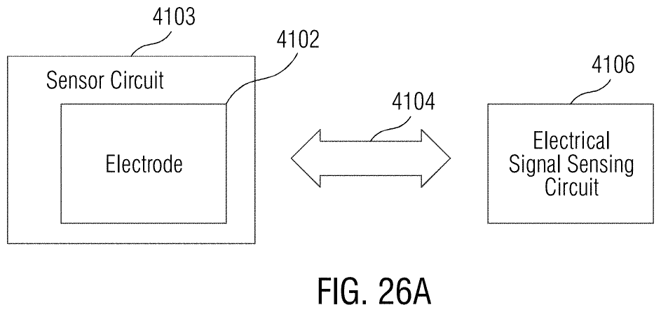

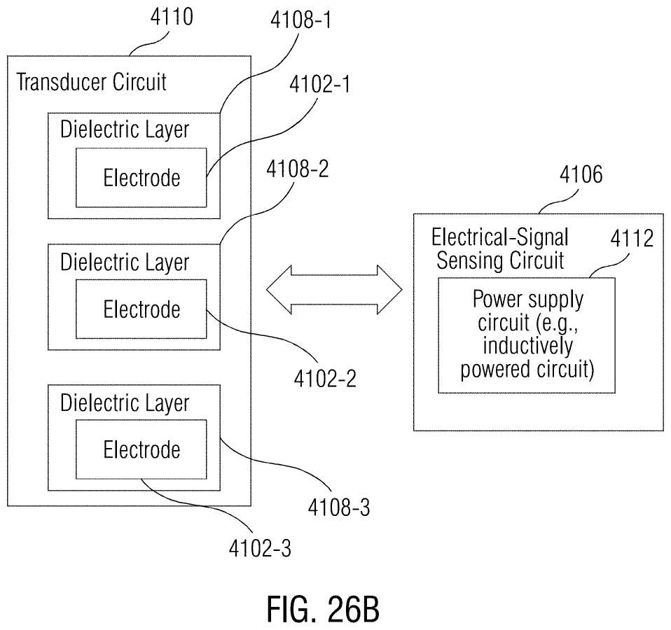

Turning now to the figures, FIGS. 26A-26B show examples of apparatuses, in accordance with the present disclosure. As illustrated by FIG. 26A-26B, each apparatus includes a sensor circuit having an electrode and an electrical-signal sensing circuit. The apparatuses can monitor pressure differences and/or capacitance changes attributable to pulse-wave events and use the monitored pressure differences and/or capacitance changes to determine one or more hemodynamic parameters. The pulse-wave events can be used to generate waveforms or parts of the waveforms responsive to or indicative of a pulse of a user or animal (e.g., represents a tactile palpation of the heartbeat). The pulse-wave event can be captured as a signal and used to determine the hemodynamic parameter, such as a heart rate, diastolic blood pressure, systolic blood pressure, and/or arterial stiffness.

FIG. 26A illustrates an example apparatus comprising a sensor circuit 4103 which includes an electrode 4102, and an electrical-signal sensing circuit 4106. The electrode 4102 can be placed near or onto the skin of a user (or another animal). The electrical-signal sensing circuit 4106 can include a proximity electrical-signal sensing circuit that senses pulse-wave events while the electrode 4102 is placed near the skin or onto the skin of the user. In some aspects, as further illustrated herein, the electrode 4102 can be in direct contact with the skin or can be electrically or mechanically isolated from the skin, such as by air or dielectric material. The electrode 4102 is used to sense pressure and/or capacitance changes that are attributable to pulse-wave events and output a signal indicative of the sensed pressure or capacitance changes to the electrical-signal sensing circuit 4106 via the sensor circuit 4103 and a communication path 4104 (e.g., the electrode is connected to or plugged-in to the sensor circuit 4103 which captures and outputs the signal indicative of a capacitance value). The electrical-signal sensing circuit 4106 monitors the changes in pressure or capacitance (or relative capacitance changes) attributable to the pulse-wave events and determines a hemodynamic parameter, such as a heartrate, from the same. The changes in pressure and/or capacitance can be measured based on relative changes in capacitance which may be caused by changes in distances between the electrode 4102 and the skin of the user and/or changes in the electric fields around the blood vessels.

The sensor circuit 4103 and/or the electrode 4102 (or plurality of electrodes), in specific aspects, are mechanically constrained to the skin or other body portion, such as by a wristband or a piece of clothing. The mechanical constraint can be via an elastic, flexible or bendable band and/or an adhesive that attaches the sensor circuit 4103 and/or electrode 4102 to the skin or body. The adhesive can be applied to the perimeter of the sensor circuit 4103 (and not necessarily between the electrode 4102 and the skin or other body portion). In other aspects, the electrode 4102 does not physically touch the skin, such as via spacers or otherwise, as described further herein. The capacitance changes of interest can be relative and not absolute values. The base capacitance of the electrode 4102 can depend on the respective geometry and range of electrode 4102 and/or the sensor circuit 4103 design. In an example experimental aspect, the electrical-signal sensing circuit 4106 can measure an input range (e.g., capacitance changes) of plus and minus 15 picofarad (pF) with a maximum offset of 100 pF. The base capacitance can be on the order of 5-75 pF and the resulting pulse-waveform signal (from the pulse-wave events) can have a maximum amplitude on the order of 0.1 to 1 pF. However aspects are not so limited and such values can be revised for different applications through the sensor and electronic designs.

The relative changes in capacitance over a period of time can be used to generate and/or otherwise output a pulse-waveform signal. The pulse-waveform signal can be indicative of the hemodynamic parameters and/or can include an arterial pulse-wave (or sometimes referred to as an “arterial pressure wave”). The changes in capacitance that are attributable to the pulse-wave events can be used to determine hemodynamic parameters, such as a heart rate, diastolic blood pressure, systolic blood pressure, mean arterial pressure, and/or arterial stiffness. As may be appreciated by one of ordinary skill in the art, an arterial pulse-waveform is a wave shape generated by the heart when the heart contracts and the wave travels along the arterial walls of the arterial tree. Generally, there are two main components of this wave: a forward moving wave and a reflected wave. The forward wave is generated when the heart (ventricles) contracts during systole. This wave travels down the large aorta from the heart and gets reflected at the bifurcation or the “crossroad” of the aorta into 2 iliac vessels. In a normal healthy person, the reflected wave can return in the diastolic phase, after the closure of the aorta valves. The returned wave has a notch and it also helps in the perfusion of the heart through the coronary vessels as it pushes the blood through the coronaries. The velocity at which the reflected wave returns becomes very important: the stiffer the arteries are, the faster it returns. This may then enter into the systolic phase and augment final blood pressure reading. The arterial pulse-wave travels faster than ejected blood.

The example apparatus illustrated by FIG. 26A (as illustrated by FIG. 268) can be modified in a variety of ways, such as illustrated by FIG. 26B. One example modification includes modifying the electrode 4102 to be electrically insulated from the skin of the user. The electrode 4102 can be insulated by adding a dielectric layer to a portion of and/or surrounding (e.g., encapsulating) the electrode 4102. In some specific aspects, the electrode 4102 can be connected to circuitry (e.g., a sensor circuit, such as a circuit board or chip) that is contained in a wristband. The electrode 4102 can be flexible. For example, the electrode 4102 can be bent around and hidden inside the wristband while worn by a user. In other examples and/or in addition, the electrode 4102 can be integrated and/or embedded into the wristband. The dielectric layer can be formed of a variety of different dielectric (or insulating) materials, such as polyester (e.g., polyethylene terephthalate), polyolefin, fluoropolymer, polyimide, polyvinylchloride, cellulose, paper, cloth, and/or other insulating material. Further, the dielectric layer can have different thicknesses, such as on the order of 5 to 250 microns. Although aspects are not so limited, and the dielectric layers can be thicker or thinner to affect the stiffness and/or comfortability of wearing the apparatus for the user or to modulate the sensitivity of the sensor circuit 4103.

The shape of the pulse-waveform can be different for different users and/or based on the location of the measurement. For example, a wider pulse pressure can suggest or be indicative of aortic regurgitation (as in diastole, the arterial pressure drops to fill the left ventricle though the regurgitating aortic valve). A narrow pulse pressure can be indicative of cardiac tamponade, or any other sort of low output state (e.g., severe cardiogenic shock, massive pulmonary embolism or tension pneumothorax). Further, the shape of the pulse-waveform can adjust depending on the location of the measurement, such as the further away the measurement is from the aorta (e.g., the brachial artery, the radial artery, the femoral artery, and the dorsalis pedis). However, with the changes in shape of the waveform, Mean Arterial Pressure (MAP) may not change and/or changes within a threshold amount. This is because, from the aorta to the radial artery, there is little change in the resistance to flow. MAP begins to change once the location is moved to the arterioles. The change in shape from the aorta location to the dorsalis pedis can include an increase in systolic peak, dicrotic notch that is further away from the systolic peak, lower end-diastolic pressure (e.g., wider pulse pressure), and later arrival of the pulse (e.g., sixty millisecond delay in radial artery from aorta). The resulting shape is sometimes called distal systolic pulse amplification as the systolic peak is steeper and further down the arterial tree.

Aspects in accordance with the present disclosure are used to output pulse-waveforms and to determine various hemodynamic parameters using an apparatus including a wearable apparatus including a sensor circuit that is non-invasive. The apparatus can be used to monitor heart rate, diastolic blood pressure, systolic blood pressure, arterial stiffness, blood volume, and other parameters. Previous invasive apparatuses, such as an arterial line, are medically inserted into a user, which can be painful, restricts patient movement, and can put the user at risk for infection and other complication. For example, an arterial line is a thin catheter inserted into an artery of the user. Often the catheter is inserted into the radial artery of the wrist but can also be inserted into the brachial artery at the elbow, the femoral artery in the groin, the dorsalis pedis artery in the foot, and/or the ulnar artery in the wrist. Arterial lines can be used in intensive care medicine and anesthesia to monitor blood pressure directly and in real-time. As insertion can be painful, an anesthetic (e.g., lidocaine) can be used to make the insertion more tolerable and to help prevent vasospasm. Complications from arterial lines can lead to tissue damage and even amputation. The apparatus in accordance with the present disclosure can be used to monitor blood pressure in real-time in a non-invasive manner. The apparatus can avoid and/or mitigate risk caused by invasive devices, such as temporary occlusion of the artery, pseudoaneurysm, hematoma formation or bleeding at the puncture sites, abscess, cellulitis, paralysis of the median nerve, suppurative thromobarteritis, air embolism, compartment syndrome and carpal tunnel syndrome, nerve damage, etc.

As illustrated by FIG. 26B, various characteristics can be revised to adjust the sensitivity of the apparatus and/or improve signals obtained by the electrode. FIG. 26B illustrates an example apparatus comprised of a plurality of electrodes 4102-1, 4102-2, and 4102-3. Each electrode 4102-1, 4102-2, 4102-3 is used to sense pressure or capacitance changes attributable to pulse-wave events (e.g., caused by changes in distance between an electrode and the skin surface), as previously described. The electrodes 4102-1, 4102-2, and 4102-3 can be part of or form a transducer circuit 4110 that provides the one or more signals to the electrical-signal sensing circuit 4106. The electrodes 4102-1, 4102-2, and 4102-3 can be placed at different locations of the apparatus to improve positional accuracy and/or to provide one or more reference signals for differential analysis. In some aspects, each electrode 4102-1, 4102-2, 4102-3 provides a signal indicative of pressure or capacitance changes (attributable to pulse-wave events) to the electrical-signal sensing circuit 4106. In specific aspects, the transducer circuit 4110 can have a floating ground. In other specific aspects, at least one of the sensor circuits has a floating ground (e.g., two sensor circuits, each having floating grounds, all the sensor circuits with each having floating grounds, etc.) Further, both the transducer circuit 4110 and at least one of the sensor circuit can have a floating ground.

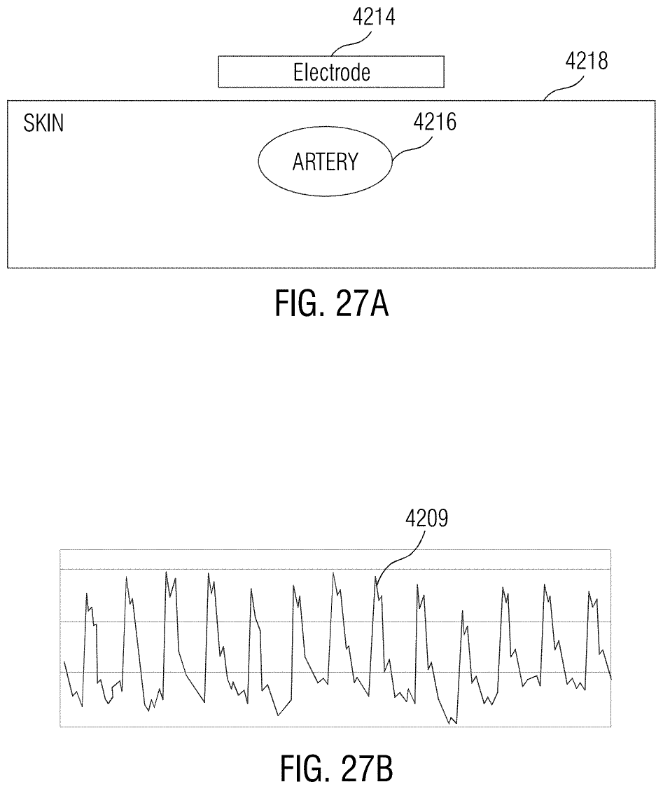

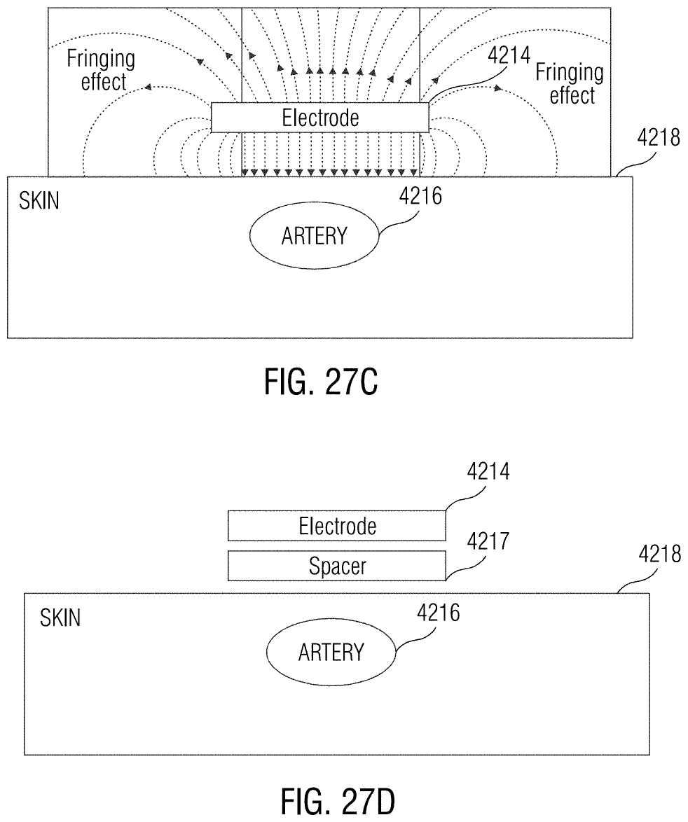

Although FIG. 26B (as well as other illustrations including but not limited to FIGS. 27A, 27B and 27D) does not illustrate a sensor circuit connected to the electrode and/or a sensor circuit connected to each of the plurality of electrodes, one of ordinary skill may appreciate that in accordance with various aspects, each electrode is connected to a sensor circuit, as previously described. In this manner, the illustration of FIG. 26B, as well as other illustrations, does not show the sensor circuit for clarity purposes and which is not intended to be limiting.

In various aspects, the apparatus (e.g., the electrical-signal sensing circuit 4106) can further include a wireless communication circuit. The wireless communication circuit wirelessly communicates data from the electrical-signal sensing circuit 4106 to circuitry that is external from the apparatus. The communication circuit can be configured and arranged to communicate the captured changes attributable to the hemodynamic pulse wave events to external processing circuitry. The communication circuit may be within or outside the wearable device and/or apparatus, and may respond to the electrical-signal sensing circuit by sending hemodynamic-monitoring data to an external circuit. Further, the apparatus can include a power supply circuit 4112, as further described herein.

In some aspects, one or more of the plurality of electrodes 4102-1, 4102-2, 4102-3 can be electrically insulated from the skin of the user. As described above, the electrodes 4102-1, 4102-2, 4102-3 can be insulated by adding a dielectric layer 4108-1, 4108-2, 4108-3 to some or all of the plurality of electrodes 4102-1, 4102-2, 4102-3. The dielectric layers 4108-1, 4108-2, 4108-3 can surround each of the electrodes 4102-1, 4102-2, 4102-3 and/or the respective sensor circuits. However, aspects in accordance with the present disclosure are not so limited and can include a dielectric layer that is position at a portion of and/or area of the electrode that is arranged to contact the skin surface, and/or surrounds at least part of the respective electrode or the sensor circuits.

The transducer circuit 4110, as illustrated by FIG. 26B, can be used to provide a differential mode to subtract out artifacts. The artifacts can be baseline shifts due to motion of the user, such as limb movement, breathing and/or changes in body temperature. In various aspects, the different electrodes 4102-1, 4102-2, and 4102-3 of the transducer circuit 4110 have different structural properties and/or characteristics used to modify the sensitivity level of the respective sensor circuit that includes the electrode. For example, the electrodes 4102-1, 4102-2, and 4102-3 can be different shapes (e.g., geometries), be positioned at different locations with respect to the user and/or the apparatus, and can be formed of different materials. In other aspects, the different structural properties and/or characteristics can include different compositions, structural components, textures, and/or thickness of the encapsulants used to electrically isolate the electrodes. For example, the dielectric layers of respective electrodes 4102-1, 4102-2, 4102-3 can be formed of dielectric material of different compositions, structures, and/or thickness to modify the sensitivity levels and/or shielding features used to isolate the electrodes. Thereby, the plurality of electrodes may have encapsulants configured and arranged to set a sensitivity level of each of the plurality of electrodes.

In various aspects, the apparatus further includes a power supply circuit 4112. The power supply circuit 4112 provides power to at least the electrical-signal sensing circuit 4106. In some specific implementations, the power supply circuit 4112 is a passively or inductively powered circuit, such as an inductor circuit. Example power supply circuits may include a battery, solar power converter, electromechanical systems, a wall plug-in (e.g., mains electricity), among other sources of power. It is possible to use energy harvesting mechanisms which capture mechanical vibrations, thermal gradients, ambient or transmitted radiation (e.g. RFID, BlueTooth, WiFi, UHF and other beacon technologies) for battery-free operation. In some implementations, the power supply circuit may include an inductive charging sub-circuit to charge a rechargeable battery. Care may be required to isolate the inductive charging sub-circuit to prevent heating due to coupling to another portion of the electronic circuit.

FIG. 27A illustrates an example of an apparatus including a sensor circuit having an electrode 4214 that interacts with skin 4218. As previously described, the sensor circuit and electrode can carry capacitance changes through proximity sensing of the skin of the user (as opposed to physically deforming as a capacitance sensor), and thereby acts as or is a proximity sensor. It has been discovered that a (proximity) sensor circuit with a single electrode 4214 placed near an arterial pulse point (e.g., artery 4216) can be used to measure the arterial pulse-waveform via the capacitance changes. Heart rate and other hemodynamic parameters may be extracted from this waveform. The electrode 4214 can be in direct contact with the skin 4218 or electrically insulated or isolated from the skin 4218. It does not need to be mechanically coupled to the skin 4218. The composition, structure and thickness of the electrical insulation can be chosen to modify the sensitivity of the sensor. Spacer structures can be used to control the distance between the electrode and the skin. The circuit may have a floating ground (e.g., the sensor circuit and/or transducer circuit can have a floating ground).

Arrays of electrodes can also be used to improve positional accuracy and/or to provide reference signals for differential analysis. And, the signal can be improved through electrode designs that optimize the fringe field distribution. For example, in some aspects, analog responses are sensed by the array of sensor circuits, with each sensor circuit having a single electrode. The two or more of the electrodes in the array can have different sensitivity levels and the analog responses sensed by the two or more sensor circuits of the arrays can be used for differential sensing.

FIG. 27B illustrates an example of a pulse-waveform 4209 that is sensed using the apparatus illustrated by FIG. 27A. As illustrated, the periodicity of the pulse-waveform 4209 reflects the cardiac cycle and can be used to determine a heart rate of the user.

FIG. 27C illustrates an example mechanism for an apparatus to monitor pulse-wave events. As illustrated by FIG. 27C, the skin 4218 of the user serves as the ground plane for the mechanism. Without constraint to a particular theory, it is believed that the mechanisms behind aspects of one or more of the aspects discussed in the disclosure are as follows: (i) the skin 4218 serves as a ground plane and arterial pressure variations cause displacement of the surface of the skin 4218, and this changes the distance between the electrode 4214 and the skin 4218 which is measured as a change in capacitance; (ii) the potential of the blood in the artery 4216 (and the overlying skin) changes with each heartbeat, and this modifies the fringe field lines which is reflected as a change in impedance; and (iii) a combination (contribution) from each of the above mechanisms is involved.

FIG. 27D illustrates an example of an apparatus, as illustrated by FIG. 27C, which further includes one or more spacers that set the distance (e.g., the minimum distance) between at least a portion of the sensor circuit (e.g., the electrode 4214) and the skin 4218. The spacer 4217 includes one or more structures formed of a material, in which the length (e.g., the distance from the electrode to the skin surface) sets the distance between at least a portion of the sensor circuit/electrode and the skin. The length can be in a range of 0.1 millimeter (mm) to 1.0 mm, although aspects are not so limited. Although the aspect of FIG. 27D illustrates one spacer having rectangular shape, aspects are not so limited and can include more than one spacer and different shaped spacers, such as a layer of textured and/or structured material.

FIG. 28 is a block diagram that exemplifies an example way for implementing the electronics and/or signal flow, from the apparatus (including, e.g., sensor circuit 4324, transducer circuit 4326, electrical-signal sensing circuit 4327 and communication circuit 4330) situated at or near the users skin to a remotely/wirelessly-communicative transceiver and CPU 4334 (e.g., received via antenna 4336), in accordance with the present disclosure. The CPU 4334 and/or electrical-signal sensing circuit 4327 can be programmed to carry out operations as disclosed herein including without limitation: processing the raw data for indicating the presence of specific hemodynamic signals, developing waveforms from the raw data, and/or evaluating the integrity, quality and relevance of the hemodynamic signals and/or waveforms for specific applications pertinent to the users hemodynamic state or well-being (the users heart rate or other hemodynamic indicators or parameters such as diastolic blood pressure, systolic blood pressure, arterial stiffness, and blood volume, and/or indicative of changes in one or more of the indicators or parameters).

The electrode of the sensor circuit 4324 captures capacitance changes responsive to pulse-wave events and provides the capacitance changes to a transducer circuit 4326. In some aspects, the transducer circuit 4326 is or includes a capacitance-to-digital converter. The capacitance-to-digital converter converts the capacitance values (e.g., the relative changes) to a digital signal and outputs the digital signal to the electrical-signal sensing circuit 4327, which can include or be a microcontroller or other processing circuitry. The electrical-signal sensing circuit 4327, using power provided by the power supply 4328, measures and/or records an arterial pulse-waveform and optionally conditions the signal, evaluates the quality of the data, and/or determines one or more hemodynamic parameters. The electrical-signal sensing circuit 4327 can output the waveform and other optional data to the CPU 4334 via the communication circuit 4330 (e.g., the integral transceiver) and antenna 4332.

The sensing apparatuses, as described herein, can be used to monitor pulse-wave events in a hands-free manner and without interference from environmental noise (e.g., human voices and other background noise, electrical interference, and/or ambient light). Moreover, the electrical-signal sensing circuit can sense the hemodynamic or pulse-wave events in response to electrical signals from the transducer circuit. The electrode (or array of electrodes) can consume relatively low amounts of power (e.g., between (less than) 5 microwatts and 3 milliwatts). In some specific aspects, the power consumption can be further reduced by only saving data after a trigger event and/or transmitting saved data in burst transmissions. Trigger events can include particular heart events that may be indicative of a problem, such as heart rates above or below a threshold amount and/or particular waveform characteristics.

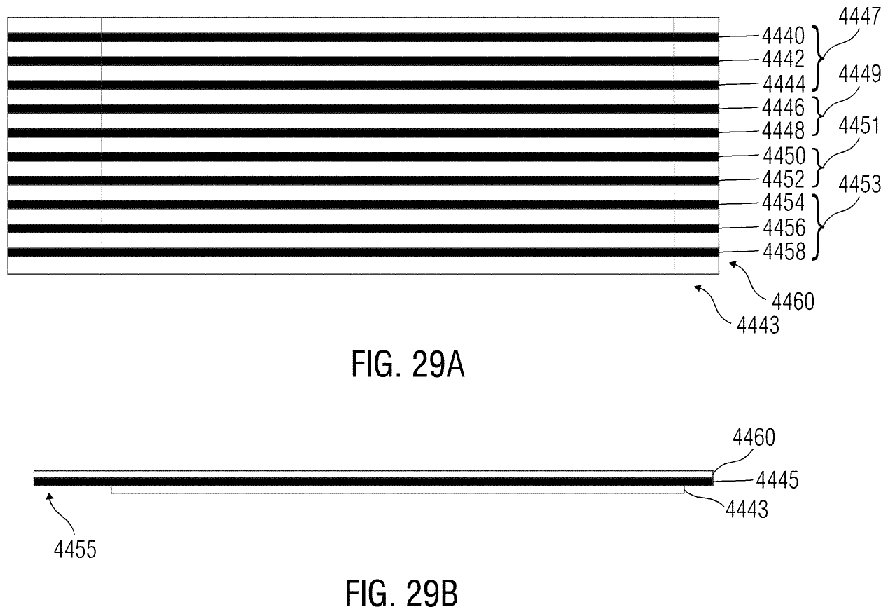

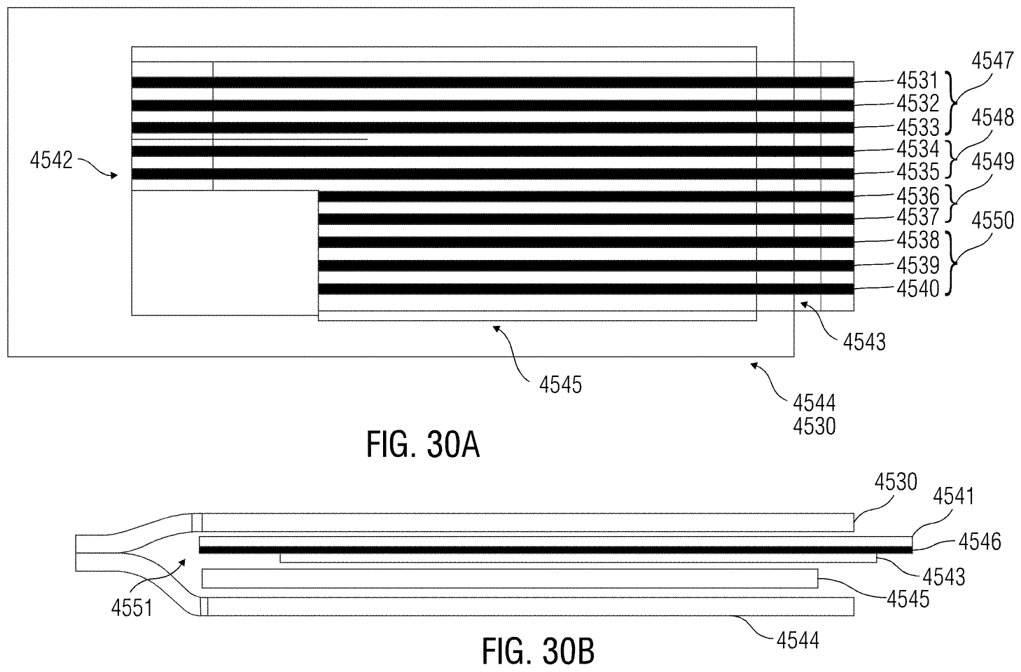

FIGS. 29A-30B illustrate various example apparatuses having an array of sensors, in accordance with the present disclosure. For example, FIGS. 29A-29B illustrate an example apparatus with four electrodes configured to interact with skin of the user.

FIG. 29A illustrates a top-down (or birds-eye) view of an apparatus comprised of a sensor array having four sensor circuits including four electrodes 4447, 4449, 4451, 4453. The line widths and spacings can be on the order of 0.1 mm to 20 mm for pulse monitoring applications. As illustrated, the sensor array includes optional ground connections 4440, 4458 and optional active shield connection 4442, 4448, 4450, 4456. The array of sensors further includes sensor connections 4444, 4446, 4452, 4454 and insulation layers 4460, 4443.

FIG. 29B illustrates a side view of the apparatus illustrated by FIG. 29A. As illustrated, the layers include the insulating layer 4460, the four electrodes 4445 (e.g., the electrodes 4447, 4449, 4451, 4453 illustrated by FIG. 29A), and another insulating layer 4443. The apparatus includes an active portion (or region) 4455 configured to be in proximity to or in contact with the skin of a user or other subject. The length of the active portion 4455 can be on the order of 0.1 mm to 20 mm or more for pulse monitoring applications. Further, the active portion 4455 can be in contact with the skin or not in contact and up to a distance of 1 mm away from the skin. In various specific aspects, the distance is typically less than 100 microns from the skin, which can be a distance sufficient to obtain signals having resulting signal-to-noise values that are high enough to obtain heart rate and/or blood pressure therefrom. In specific aspects, the electrodes 4445 can be textured or corrugated for sensitivity purposes and to reduce the contact with the skin. Smaller active areas may have higher sensitivities but can be difficult to position accurately.

In various aspects, the apparatus include a packaged array of sensors including the (four) electrodes 4445 configured to interact with the skin of the user. The array of sensors (e.g., the electrodes) can be packaged in insulating material (e.g., dielectric material) to provide environmental stability and resistance to moisture. The insulating material can include polyester, polyolefin, fluoropolymer, polyimide, polyvinylchloride, cellulose, paper, cloth, among other material. The packaging thickness can be on the order of 5 to 250 microns or more. Similarly, optional adhesive and conductive layer thicknesses can be on the order of tens of microns, and typically less than 70 and 5 microns for adhesive and the conductive layer respectively. The conductive layer(s) can optionally be passive shielding layers and/or connected to the control electronics to provide active shielding.

In specific aspects, the layers include the insulating layer with optional shielding and adhesive coatings, an insulating layer, one or more electrodes, another insulating layer, and another insulating layer with optional shielding and adhesive coatings, as further illustrated and discussed herein in connection with FIGS. 30A-30B.

In some aspects, the array of sensors (e.g., the electrodes) can be packaged in insulating material (e.g., dielectric material) to provide increase environmental stability and resistance to moisture. One or more insulating layers can be slit at one or more locations to mechanically isolate individual sensor circuits and to increase the conformability of the packed sensors to the underlying substrate.

In further specific aspects, the packaged array of sensors include the (four) electrodes 4445 and a spacer layer. The spacer layer includes one or more spacers that can set or control a distance of the sensor circuits and/or the electrodes (or at least a portion thereof) from the skin surface, as previously illustrated by FIG. 27D. The spacer layer can minimize or mitigate stray capacitance from non-active (non-sensor) regions. The spacer layer thickness can be on the order of 0.1 mm to 5 mm or more, so long as the distance does not impact sensor sensitivity. The array of sensors (e.g., the electrodes) can be packaged in insulating material (e.g., dielectric material) to provide environmental stability and resistance to moisture. The packaging thickness can be on the order of 5 to 250 microns or more. Similarly, optional adhesive and conductive layer thicknesses can be on the order of tens of microns, and typically less than 70 and 5 microns for adhesive and the conductive layer respectively.

The packaged array of sensors can further include a shield layer. As further described below, one or more insulation layers can have an adhesive coating on its inner surface such that the layer adheres to other layers, such as adhering the insulation layer to another insulation layer(s). The insulation layer can have a conductive layer on its outer surface that contacts the skin of the user. The conductive layer may alternatively be sandwiched between two insulating layers. The conductive material can include, for example, aluminum, gold, carbon, or copper that has been printed, evaporated, sputtered, or plated onto a non-conductive substrate (e.g., of PET or polyimide substrate). The insulating layers can be slit at one or more locations to mechanically isolate individual sensor circuits and to increase the conformability of the packaged sensor circuits to the underlying substrate. Thereby, the substrate can be configured and arranged as a user accessory conforming to the user's wrist, limb, or other body portion.

In a specific experimental aspect, the insulating layers 4443, 4460 and the electrodes 4445 are formed of flexible flat cable (FFC/FPC) cable (such as commercially available Molex 15168-0147), the insulating layer 4460 with the adhesive coating is formed of Polyethylene terephthalate (PET) with an adhesive (such as commercially available Avery 15660), the insulating layer 4443 with a conductive material is formed of 12 micron PET with around or greater than 2 optical density evaporated aluminum (such as commercially available Celplast Cel-Met 48g) and the spacer layer is formed of a layer of foam tape (such as commercially available Nexcare 731). Individual electrodes can be 0.625 mm wide with 0.625 mm spaces between them.

The different electrodes 4445 can have different capacitive sensitivities. The apparatus can include a spacer layer that covers active portions of some sensor circuits but not all sensors. The sensor circuits may have isolated electronics for readouts to prevent or mitigate crosstalk through a common circuit.

The flexibility or degree of bending of the sensor circuits as illustrated throughout this disclosure (e.g., including FIGS. 26A-26B, 27A, 27C-27D, 29A-29B, 30A-30B, and 31B) can be sufficient to capture the changes in pressure or capacitance (e.g., a change in capacitance values). More specifically, the degree of stiffness of the sensor circuit is inversely proportional to the thickness and/or length of the sensor circuit (e.g., the thicker or longer the electrode, the stiffer). Flexibility and thickness (and/or length) can be configured relative to one another sufficient to provide a sensitivity to pressure changes of 0.3 kilopascal (kPa) to 1 kPa and/or capacitance changes in a range of plus and minus 15 picofarad (pF) from a base capacitance of the sensor circuit. In more specific aspects, the flexibility and thickness and/or length can be configured relative to one another sufficient to provide a sensitivity to pressure changes of 0.5 kPa to 1 kPa. Further, as described herein, the measurement of changes in pressure, which are indicative of a change in capacitance, can be sensed when the sensor circuit is touching the skin or other surface. The sensed change in capacitance can be obtained when the electrode(s) are not contacting the skin or other surface of the user (but are within 1 mm away).

FIGS. 30A-30B illustrate an example apparatus having a packaged array of sensors including a plurality (e.g., four) of electrodes having different capacitive sensitivities. The apparatus includes a spacer layer 4545 that covers active portions of some sensor circuits (e.g., electrode 4547 and 4548) but not all sensor circuits (e.g., not electrodes 4549 and 4550). Alternatively and/or in addition, some of the sensor circuits (e.g., electrodes 4549 and 4550) and portions of the insulating layers 4541, 4543 are a shorter length than the remaining sensors (e.g., electrodes 4547, 4548) (with respect to the end of the apparatus proximal to the active portion 4551). The sensor circuits may have isolated electronics for readouts to prevent or mitigate crosstalk through a common circuit. As previously described, one or more insulation layers 4530 can have an adhesive coating on its inner surface such that the insulating layer 4530 adheres to other layers, such as adhering the insulation layer 4530 to other insulation layers 4541, 4544. The other insulation layer 4544 can have a conductive layer on its outer surface that contacts the skin of the user.

FIG. 30A illustrates a top-down (or birds-eye) view of the apparatus comprised of four electrodes 4547, 4548, 4549, 4550. As illustrated, the sensor array includes optional ground connections 4531, 4540, and optional active shield connections 4532, 4535, 4536, 4539. The array of sensors further includes sensor connections 4533, 4534, 4537, 4538, insulation layers 4541, 4543, a spacer layer 4545, and additional insulation layers 4544, 4530 with optional shielding and adhesive coatings. The insulating layers 4541, 4543 can be slit at one or more location(s) 4542 to mechanically isolate individual sensor circuits and to increase the conformability of the packed sensors to the underlying substrate.

FIG. 30B illustrates a side view of the apparatus illustrated by FIG. 30A. As illustrated, the layers include the insulating layer 4530 with an adhesive coating on the inner surface (e.g., on the surface proximal to the insulating layer 4541), insulating layer 4541, the four electrodes 4546 (e.g., the electrodes 4547, 4548, 4549, 4550 illustrated by FIG. 30A), another insulating layer 4543, spacer layer 4545, and another insulating layer 4544 with a conductive material on the outer surface (e.g., on the surface that is opposite and/or not proximal to the spacer layer 4545). The apparatus includes an active portion 4551, as previously described.

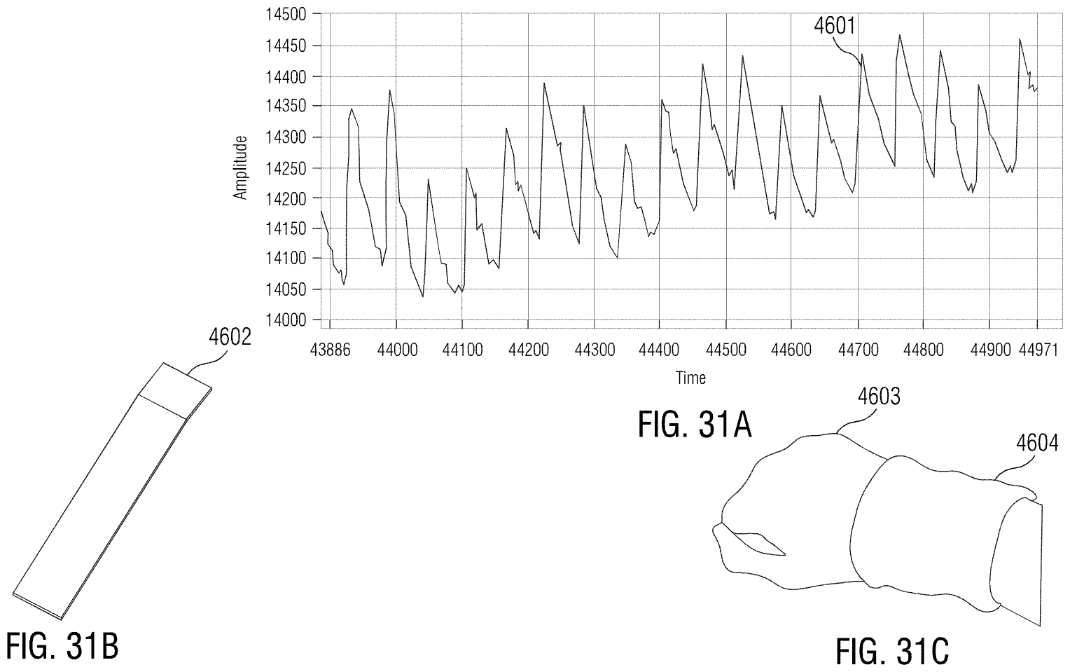

FIGS. 31A-31C illustrate an apparatus, in accordance with the present disclosure. In certain aspects, as illustrated by FIGS. 31B and 31C, the apparatus can have a flex ribbon sensor array 4602 configured and arranged to sense pulse-waveforms. The flex ribbon sensor array 4602 can be held in pace with a wristband 4604, as illustrated by FIG. 31C, that can be placed around a wrist of a user 4603. The chart illustrated by FIG. 31A shows capacitance data for a characteristic radial arterial pulse-waveform shape 4601. In an example experimental aspect, a bandpass filter (20 Hz/0.5 Hz) is used to process the data, resulting in a calculated heartrate that is 71 bpm. The reference heartrate (from a Fitbit Charge HR™) is 70 bpm, which demonstrates that the sensor signal is reflective of the cardiac cycle. For this aspect, the flex ribbon sensor array 4602 is held by an elastic wristband 4604 to contact flat next to the user's skin. A Molex 15168-0147 FFC jumper cable can be used as the flex ribbon sensor array, in various aspects. The heartrate can be calculated from a Fourier transform of such waveform data. The flex ribbon sensor array 4602 can be connected to a Bluetooth proximity sensing circuit (e.g., an electrical-signal sensing circuit).

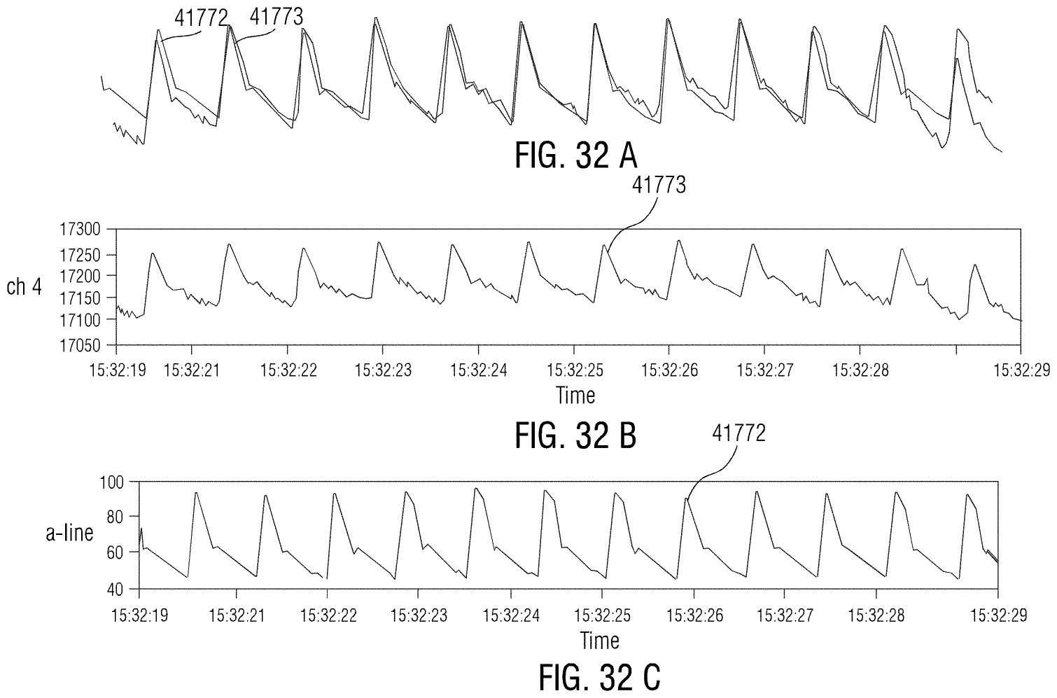

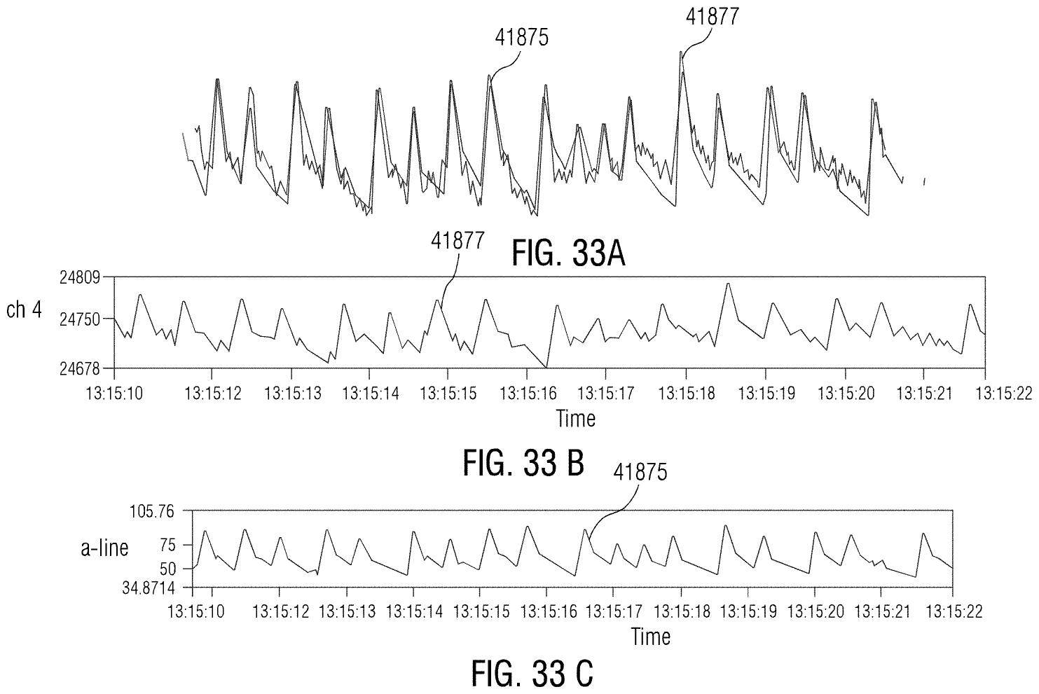

FIGS. 32A-32C illustrate example data collected using an apparatus and data collected using an arterial line, in accordance with various experimental aspects. The data obtained using an apparatus (that is placed proximal to the left radial pulse point of a user) tracks and/or mimics data obtained using an arterial line implanted in the right radial artery. FIG. 32A illustrates that the data 41773 obtained by apparatus in accordance with various aspects mimics the data 41772 obtained by the arterial line. FIG. 32B illustrates the data 41773 (e.g., the waveform) and FIG. 33C illustrates the data 41772, separately for further illustration.

FIGS. 33A-33C illustrate example pulse-waveform data collected using an apparatus and collected using an arterial line, in accordance with various experimental aspects. The data obtained using an apparatus (that is placed proximal to the left radial pulse point of a user) tracks and/or mimics data obtained using an arterial line implanted in the right radial artery. The heartrate can be determined on a beat-by-beat analysis by measuring the length of the pulse. The heartrate variability can be determined from the distribution of individual heartrate values. FIG. 33A illustrates that the pulse-waveform data 41877 obtained by apparatus can mimic the pulse-waveform data 41875 obtained by the arterial line. FIG. 33B illustrates the pulse-waveform data 41877 (e.g., the waveform) and FIG. 33C illustrates the pulse-waveform data 41875, separately for further illustration.

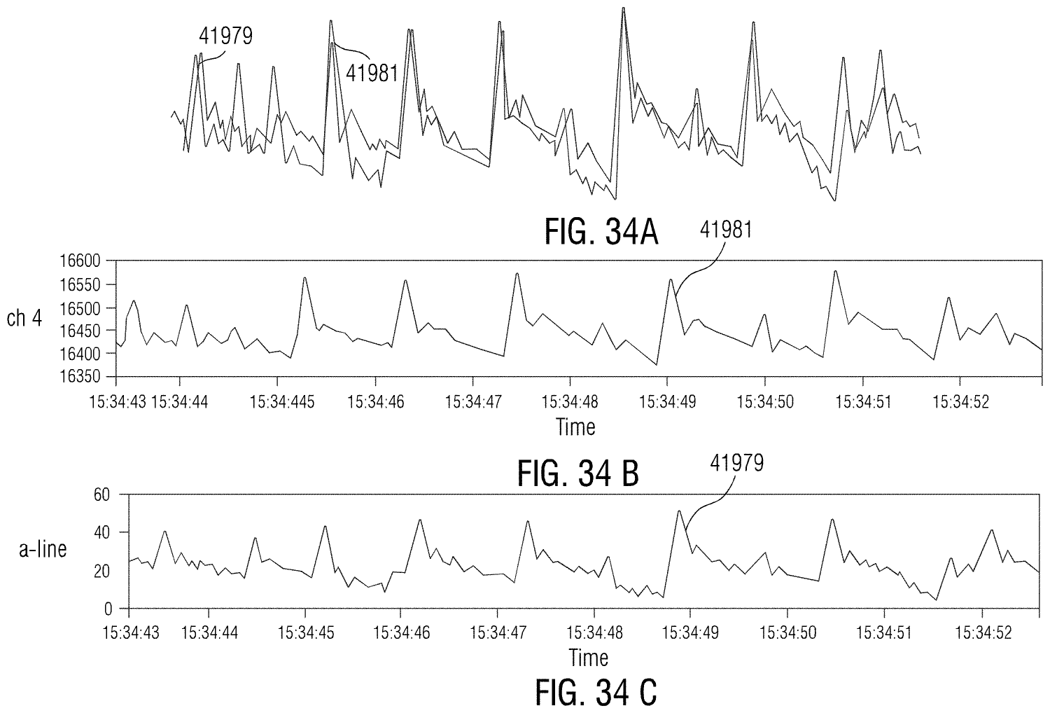

FIGS. 34A-34C illustrate example of changes in heartrate and blood pressure as collected using an apparatus and collected using an arterial line, in accordance with various experimental aspects. Patterns and anomalies in heartrate and blood pressure can be tracked and/or monitored, in various aspects. Such patterns and/or anomalies can be indicative of various health conditions, such as atrial fibrillation, hypertension, peripheral vascular disease, aortic regurgitation, aortic stenosis, and/or left ventricular obstruction, among other conditions. The data obtained using an apparatus (that is placed proximal to the left pulse point of a user) can track and/or mimic data obtained using an arterial line implanted on the right radial artery. FIG. 34A illustrates that the data 41981 obtained by apparatus in accordance with various aspects mimics the data 41979 obtained by the arterial line. FIG. 34B illustrates the data 41981 (e.g., the waveform) and FIG. 34C illustrates the data 41979, separately for further illustration.

As illustrated and previously described, the pulse-waveform can be used to determine various hemodynamic parameters. For example, the shape and other features of the pulse-waveform can be correlated to blood pressure. In other aspects, the heart rate and heart variability can be obtained by determining the timings of each pulse. Further, the changes in blood pressure can be monitored by first calibrating the data (such as with arterial lines that are calibrated against inflatable cuff data).

Various different techniques can be used to analyze the pulse-waveform and/or to determine various hemodynamic parameters including feature analysis and computation fluid dynamics techniques. For example, features attributed to hemodynamic phenomena can be correlated to blood pressure, arterial stiffness, and other hemodynamic parameters. For more general and specific information on features attributed to hemodynamic phenomena, reference is made to Cecelia, Marina, and Phil Chowienczyk. “Role of Arterial Stiffness in Cardiovascular Disease.” JRSM Cardiovascular Disease 1.4 (2012): cvd.2012.012016, PMC, Web. 31 Jan. 2017; David A. Donley et al, “Aerobic exercise training reduces arterial stiffness in metabolic syndrome” Journal of Applied Physiology published 1 Jun. 2014, Vol. 16, no. 11, 1396-1404; Baruch, Martin C, et al “Validation of the pulse decomposition analysis algorithm using central arterial blood pressure.” Biomedical engineering online 13.1 (2014): 96, and Munir, Shahzad, et al. “Peripheral augmentation index defines the relationship between central and peripheral pulse pressure.” Hypertension 51.1 (2008): 112-118, each of which are fully incorporated herein. As another example, the augmentation index (AI), (peripheral second systolic blood pressure (pSBP2)-diastolic blood pressure (DBP))/(peripheral systolic blood pressure (pSBP)-DBP) can be used as a marker for arterial stiffness and may be correlated to both peripheral and central peak blood pressure (pPP & cPP). AI is a normalized parameter and can be analyzed without absolute calibration. Computation fluid dynamic techniques can include modeling vasculature as an inductor capacitor resistor (LCR) circuit and/or as a network of elastic pipes to calculated parameters such as pulse-wave velocity and/or waveform shape. For more general and specific information related to computation fluid dynamics used to determine hemodynamic parameters, reference is made to Lee, Byoung-Kwon. “Computational fluid dynamics in cardiovascular disease.” Korean circulation journal 41.8 (2011): 423-430, and Xiaoman Xing and Mingshan Sun, “Optical blood pressure estimation with photoplethysmography and FFT-based neural networks,” Biomed. Opt. Express 7, 3007-3020 (2016), each of which are fully incorporated herein by reference. One model that can be used to derive a relationship between the pulse-waveform (obtained by PPG) and blood pressure, where g is defined by the modulus E of the blood vessel walls, includes:

E=E₀e^{γP}

For example, a normalized waveform can be given by:

$$\begin{matrix}= \frac{V - {V}_{min}}{{V}_{min}} \\ \frac{2 ( {e}^{- Υ {P}_{min}} - {e}^{Υ P} )}{b - 2 {e}^{- {Υ P}_{min}}} \\ α k ( {e}^{- Υ {P}_{min}} - {e}^{- Υ P} )\end{matrix}$$

Various techniques can be used for correlating the pulse-waveform to blood pressure values. For more general and specific information related to correlating pulse-waveforms to blood pressure values, reference is made to Xing, Xiaoman, and Mingshan Sun. “Optical Blood Pressure Estimation with Photoplethysmography and FFT-Based Neural Networks.” Biomedical Optics Express 7.8 (2016): 3007-3020, and http://cs229.stanford.edu/proj2014/Sharath %20Ananth,Blood %20Pressure %20Detection %2 Ofrom %20PPG.pdf, each of which is fully incorporated herein by reference.

### Applications

Applications of the sensor bands 1800, 1900 apparatuses comprising proximity sensors and firmware) to detect signals from a patient's body using any one of the proximity sensors 100, 200, 300, 400, 500, 600, 700, 800, 1000, 1100, 1200, 1300 described in FIGS. 1-8, 12, 13, 15, 16, and 21-24 described herein include:

Blood pressure measurements—systolic, diastolic, mean arterial pressure, pulse pressure, and their variabilities, both as time series values and as trends;

Vascular checks looking for pulse wave or heart beat as a substitute for Doppler measurements;

Monitoring blood pressure and heart rate trends for conditions such as onset of pre-eclampsia, hypotensive crises in the ICU, pre-hospital hypotension for the mitigation of head injuries, post-hospital or home hypertension episodes, intradialytic hypotension, nocturnal hypertension, masked hypertension, atrial fibrillation, premature ventricular contractions and other heart rate irregularities, dehydration;

Circulatory comparisons with measurements at multiple pulse points, e.g. between upper and lower body, using ratios of blood pressure similar to ankle-brachial index tests or pulse height values or other metrics derived from a comparison of pulse-waveform shapes obtained at different locations, to diagnose peripheral arterial disease, vascular complications, insufficient blood flow, or heart defects such as coarctation of the aorta; and

Circulatory time analysis using trends in blood pressure or pulse height values or changes in pulse-waveform shapes to determine complications or efficacy of operative procedures.

Other more detailed information about the cardiovascular system through the pulse-waveform shape in the same way that arterial line data has been related to heart rate, heart rate variability, cardiac output, and respiratory rate.

Monitoring blood pressure and heart rate trends for hypertensive disorders such as preeclampsia affect up to 15% of pregnancies, contributing to: 2.6 million premature births, 0.5 million infant deaths, and 40% maternal deaths each year. Thus, there is an unmet need for low-cost, easy-to-use BP monitoring during the third trimester of pregnancy.

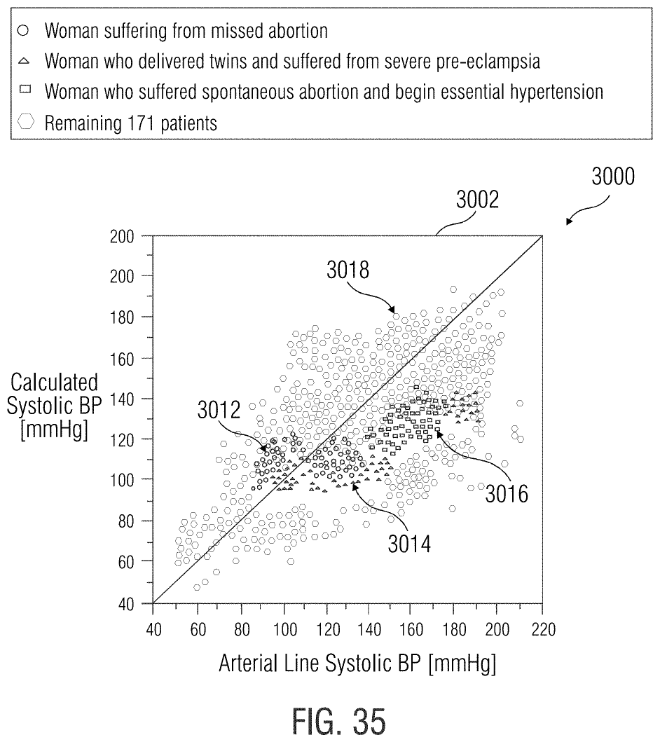

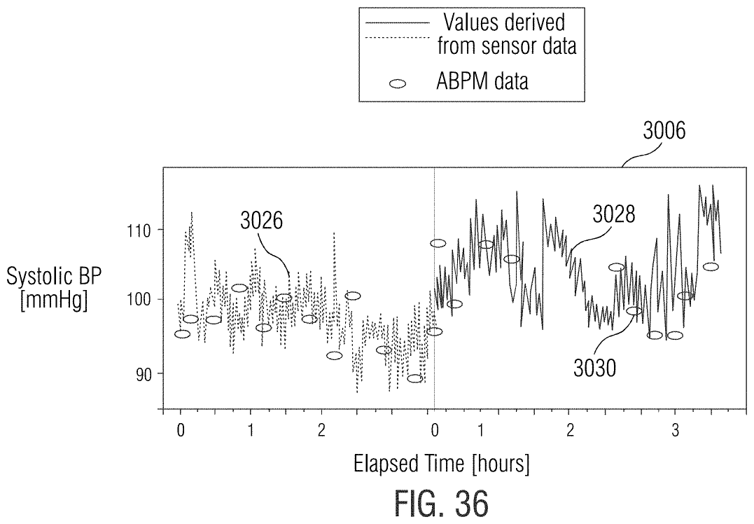

The shape of the pulse-waveform received by the data receiver circuit module 2012 from the sensor band circuit module 2008 can be used as a biomarker for some disease states, either directly or through machine learning classification models. For example, with reference to FIGS. 35 and 36, there is some evidence that there are differences between the pulse-waveform shapes of nominally healthy pregnant women versus pregnant women who have been hospitalized for complications. The algorithms executed by the data receiver circuit module 2012 extract blood pressure values from the pulse-waveform shape work moderately well for healthy pregnant women and non-pregnant women in critical care. The data for hospitalized pregnant women in critical care, however, was inconclusive, implying that there is a difference in the shape of the pulse-waveforms which might be expected since some hypertensive disorders of pregnancy are hypothesized to be due to vascular changes that occur during pregnancy.

FIG. 35 is a graph of systolic blood pressure (sBP) calculated from sensor data versus arterial line sBP, in accordance with various experimental aspect. FIG. 36 is a graph of systolic blood pressure (sBP) versus elapsed time, in accordance with various experimental aspects. With reference to FIGS. 35 and 36, A machine learning model was trained to extract systolic blood pressure (sBP) values from arterial line data curated from the MIMIC-Ill database. The training set comprised 200 longitudinal samples randomly chosen from each of 4040 critically ill patients.

In the graph 3002, sBP values determined from this model are shown for 174 critically ill women under the age of 45 years old. The highlighted data points 3012, 3014, 3016 are for the three women in this population who had diagnostic codes indicating complications of pregnancy during their hospital stay. The remaining points 3018 are for the 171 patients who did not have diagnostic codes related to pregnancy. The data points 3012 represent a woman who suffered from a missed abortion at 22 weeks gestation. She also suffered from hypertensive chronic kidney disease, CHF, and lupus. The data points 3014 represent a woman who delivered twins and suffered from severe pre-eclampsia and its complications. The data points 3016 represent a woman who suffered a spontaneous abortion and benign essential hypertension along with other issues.

According to the graph 3002, the model is unable to predict blood pressure values for the critically ill pregnant women while the derived blood pressures for the non-pregnant critically ill women meet the FDA guidelines for accuracy.

The model was also used to derive blood pressure values from the sensor data 3026, 3028 for two nominally healthy pregnant women. The results are compared against brachial cuff measurements 3030 taken simultaneously with an ambulatory blood pressure monitor (ABPM) as a function of elapsed time in 3006. Data 3026 represent a healthy woman in the third trimester and data 3028 represents a healthy woman in the 2nd trimester. In both cases, it is clear that the model provides blood pressure values that track cuff values as a function of time within the FDA guidelines for accuracy.

### Algorithms

Quality Models

With reference back to FIG. 25, the data receiver 2004 may be configured to process signals or data received from the sensor band 2002 comprising one or more proximity sensors 100, 200, 300, 400, 500, 600, 700, 800, 1000, 1100, 1200, 1300 described in FIGS. 1-8, 12, 13, 15, 16 to execute algorithms 2016 to grade signal quality, provide signal filtering, calculate quality models including regression coefficient models, pulse-waveform quality models, signal-to-noise ratio models, Kalman and particle filter models, artificial neural networks, and use calibration or anchor points as described in more detail hereinbelow.

Regression Coefficient Models:

Pulse-by-pulse synchronized arterial and sensor data are used to determine regression coefficients which can be used as a metric of sensor data quality. When an algorithm 2016 is trained on the sensor data provided 2026 by the sensor band circuit module 2008 with these regression coefficients as ground truth values, the network can be used to predict the likelihood that subsequent sensor data would correlate to arterial line data. This likelihood can be used as a quality metric to filter the sensor data that is to be fed into algorithms 2018 used to extract blood pressure and other hemodynamic values from sensor pulse-waveform data. Alternatively, it can be used to estimate the confidence level of the extracted blood pressure values.

Pulse-Waveform Quality Models:

The data receiver 2004 can be configured to train another type of quality model on quality ratings from a rubric based on features of pulse-waveforms such as the resolution of secondary peaks, signal-to-noise levels, lack of baseline variations or motion artifacts. In one example, pulse-waveforms can be visually rated with this rubric and a convolutional neural network trained on the pulse-waveform data with the ratings used as ground truth values. This model can then be used to provide quality ratings for subsequent sensor data. Like the predicted regression coefficients, the quality ratings can be used to filter sensor data for use to extract blood pressure values or to estimate a confidence level for the extracted values.

Alternatively, waveforms can be classified into different canonical shapes. Classification models can then be used to identify a class of waveform shapes for each new pulse-waveform. This classification can then be used to determine whether a blood pressure value can be extracted from that pulse-waveform and/or which model to use.

Signal to Noise Ratio Models:

The data receiver 2004 can be configured to implement digital filter processing techniques. In one aspect, another type of quality model can be based on Fourier filtering. In this case the data receiver 2004 can be configured to take the Fourier Transform of the received sensor data. Bandstop filters can be used to remove periodic noise such as breathing modes at lower frequencies and oscillatory ventilation noise at higher frequencies. As is known in the art, signal power can be calculated by identifying the primary frequency of the heart rate and integrating over that peak along with a number of higher harmonics (the signal data). The remaining data can be integrated to determine a noise power value. The ratio of signal power to noise power yields a metric that is indicative of the general quality of the sensor data. When calculated with a sliding data window, the signal-to-noise ratio (SNR) can be determined as a function of time and then used to filter/select the sensor data for further processing. Alternatively, the signal data reconstructed from the primary frequency and its higher harmonics can be used to derive blood pressure values from the BP algorithms.

Kalman and Particle Filter Models:

The data receiver 2004 can be configured to implement Kalman and particle filters such that the received sensor data can be subjected to such Kalman and particle filters to isolate pulse-waveform data from other periodic signals and other artifacts such as those due to electronic noise and motion. The isolated pulse-waveform data can be used in the BP algorithms to extract blood pressure values. The fit parameters of these models can be used as a metric for signal quality. Parameters of other oscillatory signals, e.g. respiration rate, may be useful as inputs to the BP models or as information for the medical team. In one aspect, respiration rates can be measured from motion of the body and in other aspects from the pulse waveform.

Any of the above quality models also may be used to determine what type of signal processing might be required to modify the data to improve the accuracy of the predicted blood pressure values. For example, the range of frequencies used in bandpass filters may be reduced for lower values of the quality metric to more heavily filter out motion artifacts or noisy signals. In another example, the sensor data may fall into a class of data with a secondary frequency due to breathing modes or high frequency oscillatory ventilation, and data with this frequency would be filtered out with a bandstop filter.

Blood Pressure Models

Artificial Neural Networks:

The data receiver 2004 can be configured to implement artificial neural networks (NN) to derive blood pressure values from normalized pulse-waveform shapes. Use of pretrained convolutional neural networks combined with feature-based regression models may be advantageous for this application. Inclusion of demographics such as gender, age, height and weight may also be advantageous.

The NN code can be structured in a modular way to enable easy introduction of new model parameters. Because of the high correlation of sensor data with arterial line data (e.g. FIGS. 17-19 in WO 2017/172978 A1), arterial line data can be used to augment the training set used for machine learning algorithms. The advantage of this is the breadth of available data enabling the sampling for thousands of individuals with a wide range of demographics for long time periods during which they are receiving a variety of medications and other treatments. Arterial line data taken simultaneously with sensor data can be used to derive ground truth values for the sensor data on a pulse-by-pulse basis, providing millions of data-ground truth pairs, for each individual. Data from both the arterial line and the sensor may require curation to remove artifacts due to motion, scaling errors, or signal compression errors. Arterial line data may also be curated to remove data where the position of the arterial line causes it to be underdamped or overdamped which can affect the accuracy of the reported systolic and diastolic blood pressure values. We have developed algorithms to enable the automatic detection of underdamped waveforms.

Use of Calibration or Anchor Points:

While uncalibrated models have been developed by the inventors using only normalized sensor pulse wave data as input, in some situations, it may be advantageous to use external data to improve the accuracy of the extracted blood pressure values. For example, demographic information such as age, gender, height and weight, and information on medical treatments such as high frequency oscillatory ventilation, circulatory assist devices, or dialysis, may be used to choose between models or as inputs in certain models. For neonates, birthweight or gestational age also may be used as inputs into the model.

Use of one or more inflatable cuff measurements at the beginning of sensor data collection can also be used as inputs into some models. It may also be advantageous to use periodic cuff measurements as inputs to the model during the course of sensor data collection.

The model may also include input obtained from a prescribed start-up regimen where the sensor is applied and then used in multiple positions. For example, one such regimen for a wrist-worn sensor could be to hold the arm up, down, and straight out, for a fixed period of time, e.g. 5 to 20 seconds, in each position. An altimeter can be used to tell the relative position of the sensor in these three positions to determine a calibration factor for the sensor data by applying a correction factor based on these sensor positions to the blood pressure values extracted from the sensor data.

In various aspects, the proximity sensors 100, 200, 300, 400, 500, 600, 700, 800, 1000, 1100, 1200, 1300 described in FIGS. 1-8, 12, 13, 15, 16 and/or the sensor bands 1800, 1900 described in connection with FIGS. 21-24 may be coupled to an accessory device to reduce motion artifacts such as vehicle or ventilator vibrations. The accessory device may include a pad of damping (e.g. viscoelastic) material to isolate the device from environmental motions, similar to the concept of a vibration-isolated optical bench. The damping could be frequency dependent and tailored to particular types of vibrations. The vibration damping material can dampen vibrations or mitigate motion artifacts. The vibration damping pad may be placed under the arm or leg that the sensor pad 1800, 1900 (FIGS. 21-24) is attached to or may be used as a mattress/seat pad underneath the patient.

### Methods

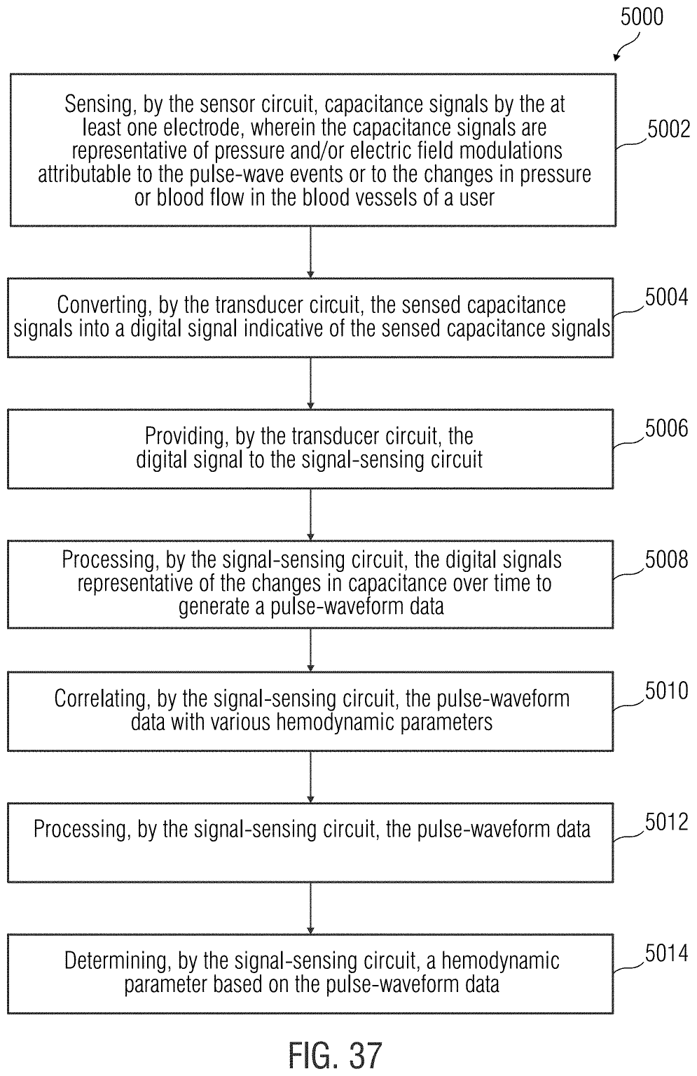

The following methods 5000, 6000, 7000 illustrated in FIGS. 37-39 can be implemented using the hardware associated with proximity sensor circuits, electrical-signal sensing circuit, and signal processing circuits described in detail hereinabove. The one or more proximity sensor circuits, electrical-signal sensing circuit, and signal processing circuits may be configured and arranged to sense hemodynamic changes (or pulse-waveforms) of a user with the sensor circuit configured in a manner to monitor the physiologic changes of the user by using a single electrode placed near/onto a surface to be measured. These and other aspects employ the proximity sensor circuits, electrical-signal sensing circuit, and signal processing circuits configured to sense the hemodynamic changes consistent with one or more of the above-described hardware and the below-described methods. Accordingly, in the description of the below-described methods, reference can be made to the hardware in description in FIGS. 1-31B, C and the data in FIGS. 31A and 32A-35.

In particular, each of the methods 5000, 6000, 7000 may be implemented by the circuit 2000 as described in FIGS. 25 and 28 coupled to any one of the proximity sensors 100, 200, 300, 400, 500, 600, 700, 800, 1000, 1100, 1200, 1300 described in FIGS. 1-8, 12, 13, 15, 16 can be employed to monitor one or more physiological parameters in a non-invasive manner and in real-time. The circuit 2000 comprises a sensor band 2002 comprising a sensor circuit module 2008 (e.g. printed circuit board assembly (PCBA) and firmware) to detect signals from a patient's body using any one of the proximity sensors 100, 200, 300, 400, 500, 600, 700, 800, 1000, 1100, 1200, 1300 (100-1300) described in FIGS. 1-8, 12, 13, 15, 16. In one aspect, the signal detected by the proximity sensor is a pulse-waveform that represents one or more physiological parameters including, for example, blood pressure, among others as described hereinbelow. In one aspect, the circuit module 2008 comprises a sensor circuit 4324 and a transducer circuit 4326 as described in FIG. 25. The sensor circuit 4324 including at least one electrode and is coupled to the transducer circuit 4326. The transducer circuit 4326 is optionally wirelessly coupled to the data receiver 2004, which comprises a circuit module 2012 that includes hardware and software to implement an electrical-signal sensing circuit 4327 to process the signals received from the transducer circuit 4326. In one aspect, the electrical-signal sensing circuit 4327 of the receiver circuit module 2012 is configured to process signals received from the transducer circuit 4326. A communication circuit 4330 can communicate to the cloud for additional processing of signals and can communicate with external monitors such as the data monitor 2006.

FIG. 37 illustrates a method 5000 for hemodynamic monitoring, in accordance with at least one aspect of the present disclosure. The method 5000 comprises hemodynamic monitoring via a wearable apparatus, such as the sensor band 2002, comprising a sensor circuit 4324 comprising at least one electrode 100-1300 placed near or onto the skin of a user, a transducer circuit 4326 to receive signals from the sensor circuit 4324 and to convert the sensed capacitance signals to digital signals and provide the digital signals to the signal-sensing circuit 4327 to process the digital signals. The method 5000 will be described hereinbelow with reference to FIGS. 25 and 28 in conjunction with FIG. 37.

According to the method 5000, the sensor circuit 4324 senses 5002 capacitance signal changes between the electrode 100-1300 and the skin of a user, wherein the capacitance signal changes are representative of pressure and/or electric field modulations attributable to the pulse-wave events or to the changes in pressure or blood flow in the blood vessels (e.g., hemodynamics). The transducer circuit 4326 converts 5004 the sensed capacitance signals into a digital signal indicative of the sensed 5002 capacitance signal changes and/or pressure and provides 5006 the digital signal to the signal-sensing circuit 4327 for digital signal processing and/or communication, for example. Due to pulse-wave events, the distance between the skin of the user and the electrode can change and/or the electric field distribution around the blood vessels can change, resulting in a relative change in capacitance as measured using the sensor circuit. The signal-sensing circuit 4327 processes 5008 the digital signals representative of the changes in capacitance over time and generates and/or determines a pulse-waveform. The signal-sensing circuit 4327 correlates 5010 the pulse-waveform data with various hemodynamic parameters, processes 5012 the pulse-waveform data, and determines 5014 heart rate, blood pressure, e.g., systolic and diastolic pressure, mean arterial pressure, pulse pressure, arterial stiffness, and/or blood volume, or combinations thereof, and their variabilities, among others, both as time series values and as trends.

In one aspect, the method 5000 comprises measuring a pulse wave or a heart beat as a substitute for Doppler measurements. In another aspect, the method 5000 comprises measuring multiple pulse points and providing circulatory comparisons. In another aspect, the method 5000 comprises determining complications or efficacy of operative procedures through circulatory time analysis using trends in blood pressure or pulse height values or changes in pulse-waveform shapes.

The method 5000 further comprises placing at least one electrode 100-1300 of the sensor circuit 4324 near or onto the skin of the user and sensing pulse-wave events. In accordance with the method 5000, the electrode 100-1300 can be in contact with the skin of the user and/or in proximity thereof. In some aspects, the electrode 100-1300 is constrained onto (whether in contact or not) the user using a mechanical constraint (e.g., a wristband, an elastically compliant band, or an article of clothing) and/or an adhesive. The electrode 100-1300 can be located near a blood vessel, preferably near a palpable pulse point such as but not limited to the radial, brachial, carotid, tibial, and temporal pulse points.

In accordance with the method 5000, the at least one sensor circuit 4324 comprises a plurality of electrodes 100-1300 arranged as part of a transducer circuit 4326 to provide electrical signals (e.g., a digital) to the electrical signal-sensing circuit 4327, wherein the electrical signal is indicative of the changes in capacitance that are responsive to modulations in distance between the skin of the user and the electrode 100-1300, pressure and/or electric field and attributable to hemodynamic or pulse-wave events. In various related aspects, a plurality of sensor circuits 4324 may be mechanically separated and/or arranged in an array (e.g., a sensor array). Each of the sensor circuits 4324 may be constructed differently, such as having different geometries, dielectric layers, locations, sensitivities, among other constructions as further described herein.

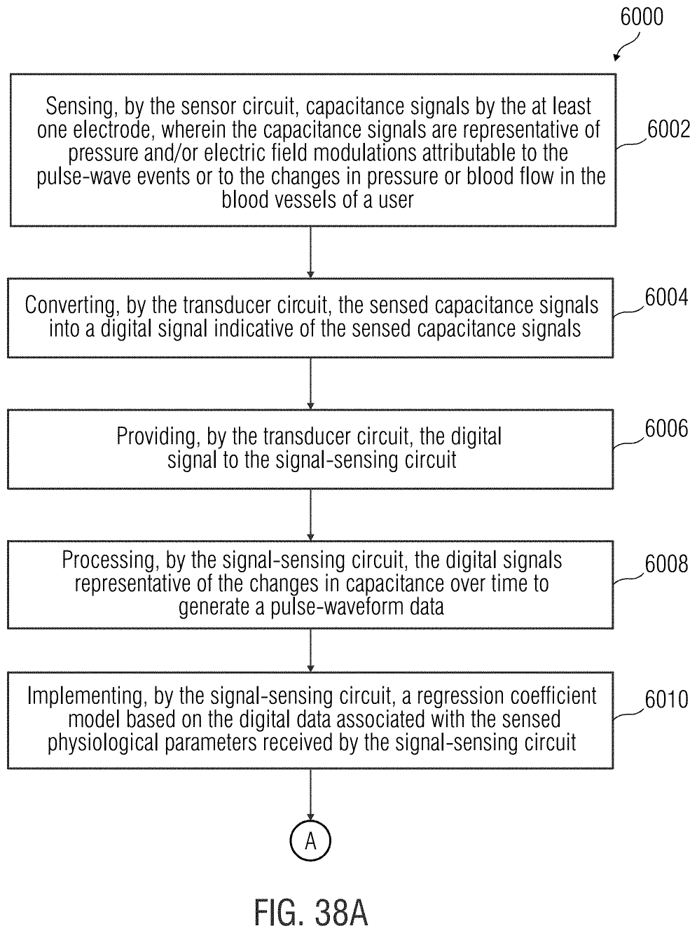

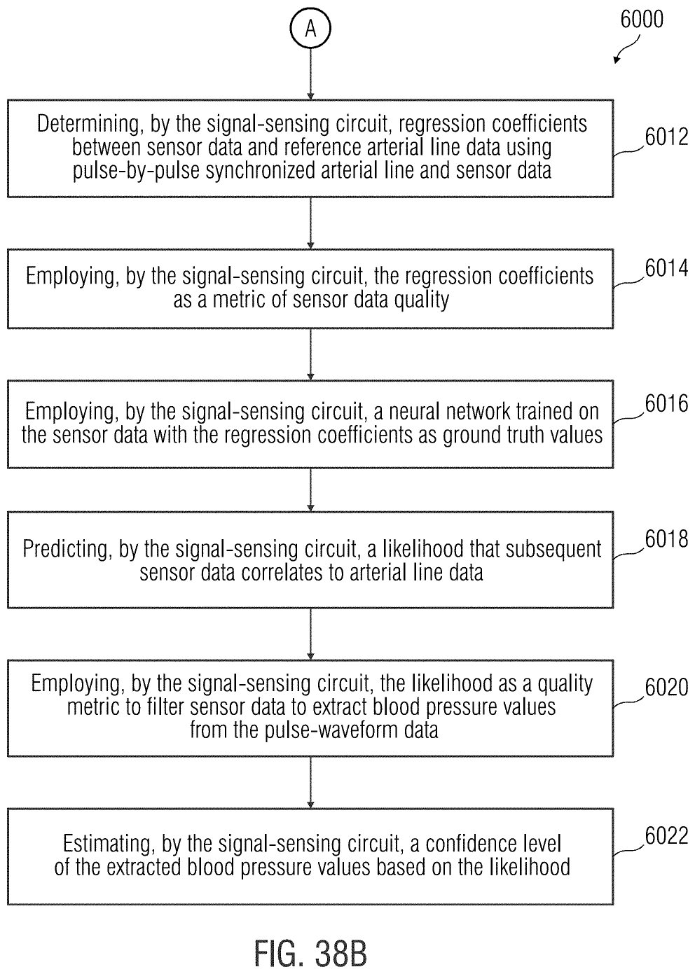

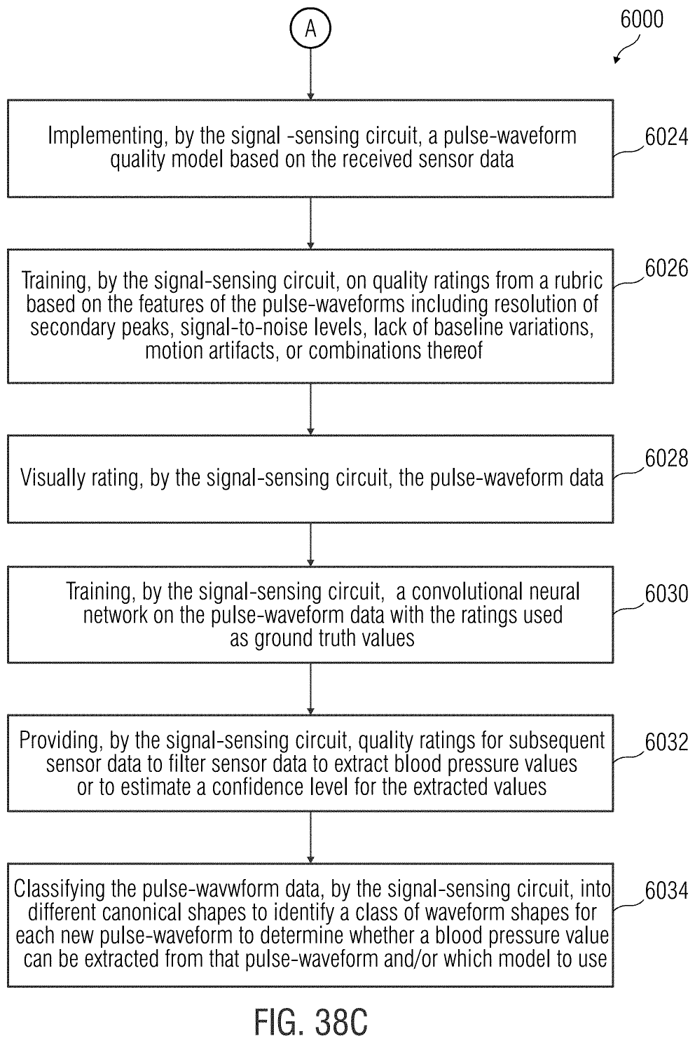

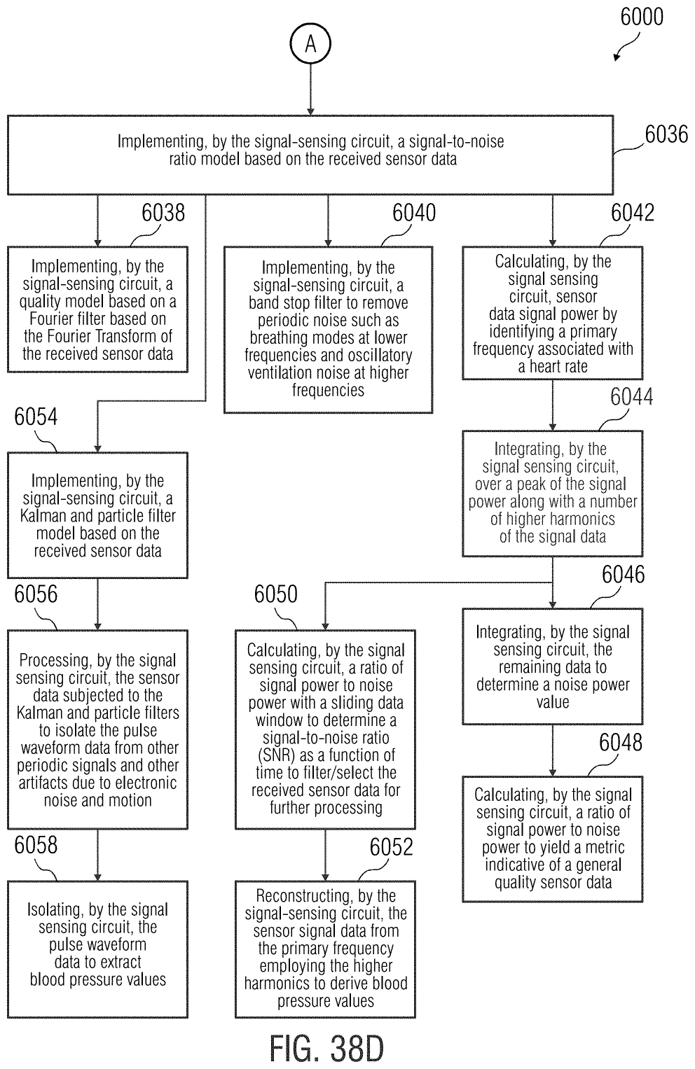

FIGS. 38A-38D illustrates a method 6000 for measuring and processing one or more physiological parameters, in accordance with at least one aspect of the present disclosure. The method 6000 comprises measuring and processing one or more physiological parameters via a wearable apparatus, such as the sensor band 2002, comprising a sensor circuit 4324 comprising at least one electrode 100-1300 placed near or onto the skin of a user, a transducer circuit 4326 to receive signals from the sensor circuit 4324 and to convert the signals to digital signals and provide the digital signals to the signal-sensing circuit 4327 to process the digital signals.

With reference to FIG. 38A, in one aspect, according to the method 6000, the sensor circuit 4324 senses 6002 capacitance signal changes between the electrode 100-1300 and the skin of a user, wherein the capacitance signal changes are representative of pressure and/or electric field modulations attributable to the pulse-wave events or to the changes in pressure or blood flow in the blood vessels (e.g., hemodynamics). The transducer circuit 4326 converts 6004 the sensed capacitance signals into a digital signal indicative of the sensed 6002 capacitance signal changes and/or pressure and provides 6006 the digital signal to the signal-sensing circuit 4327 for digital signal processing and/or communication, for example. Due to pulse-wave events, the distance between the skin of the user and the electrode 100-1300 can change and/or the electric field distribution around the blood vessels can change, resulting in a relative change in capacitance as measured using the sensor circuit 4324. The signal-sensing circuit 4327 processes 6008 the digital signals representative of the changes in capacitance over time and generates and/or determines a pulse-waveform.

With reference to FIG. 38B, in one aspect, according to the method 6000, the signal-sensing circuit 4327 receives the pulse-waveform data from the transducer circuit 4326 and implements 6010 a regression coefficient model based on the digital data associated with the sensed physiological parameters received by the signal-sensing circuit 4327. The sensed physiological parameters include heart rate, blood pressure, e.g., systolic and diastolic pressure, mean arterial pressure, pulse pressure, arterial stiffness, and/or blood volume, or combinations thereof, and their variabilities, both as time series values and as trends. The signal-sensing circuit 4327 determines 6012 regression coefficients between sensor and reference arterial line data using pulse-by-pulse synchronized arterial and sensor data. The signal-sensing circuit 4327 then employs 6014 the regression coefficients as a metric of sensor data quality.

With continued reference to FIG. 38B, in one aspect, according to the method 6000, the signal-sensing circuit 4327 employs 6016 a neural network trained on the sensor data with the regression coefficients as ground truth values. The neural network is employed by the signal-sensing circuit 4327 to predict 6018 the likelihood that subsequent sensor data would correlate to arterial line data if taken simultaneously. The signal-sensing circuit 4327 employs 6020 the likelihood as a quality metric to filter sensor data that is to be fed into algorithms to extract blood pressure values from sensor pulse-waveform data. The signal-sensing circuit 4327 estimates 6022 a confidence level of the extracted blood pressure values based on the likelihood.

With reference to FIG. 38C, in one aspect, according to the method 6000, the signal-sensing circuit 4327 implements 6024 a pulse-waveform quality model based on the received sensor data. The signal-sensing circuit 4327 trains 6026 on quality ratings from a rubric based on features of pulse-waveforms such as resolution of secondary peaks, signal-to-noise levels, lack of baseline variations, or motion artifacts, or combinations thereof. The signal-sensing circuit 4327 visually rates 6028 the pulse-waveform data and trains 6030 a convolutional neural network on the pulse-waveform data with the ratings used as ground truth values. In one aspect, according to the method 6000, the signal-sensing circuit 4327 provides 6032 quality ratings for subsequent sensor data to filter sensor data for use to extract blood pressure values or to estimate a confidence level for the extracted values. In one aspect, according to the method 6000, the signal-sensing circuit 4327 classifies 6034 the pulse-waveform data into different canonical shapes to identify a class of waveform shapes for each new pulse-waveform to determine whether a blood pressure value can be extracted from that pulse-waveform and/or which model to use.

With reference to FIG. 38D, in one aspect, according to the method 6000, the signal-sensing circuit 4327 implements 6036 a signal-to-noise ratio model based on the received sensor data. The signal-sensing circuit 4327 implements 6038 a quality model based on a Fourier filter based on the Fourier Transform of the received sensor data. In one aspect, the signal-sensing circuit 4327 implements 6040 a bandstop filter to remove periodic noise such as breathing modes at lower frequencies and oscillatory ventilation noise at higher frequencies. In one aspect, the signal-sensing circuit 4327 calculates 6042 sensor data signal power by identifying a primary frequency associated with a heart rate and integrating over a peak of the signal power along with a number of higher harmonics of the signal data. The signal-sensing circuit 4327 integrates 6044 over a peak of the signal power along with a number of higher harmonics of the signal data. The signal-sensing circuit 4327 integrates 6046 the remaining data to determine a noise power value and calculates 6048 a ratio of signal power to noise power to yield a metric indicative of a general quality sensor data. In one aspect, the signal-sensing circuit 4327 calculates 6050 a ratio of signal power to noise power with a sliding data window to determine a signal-to-noise ratio (SNR) as a function of time to filter/select the received sensor data for further processing. In one aspect, the signal-sensing circuit 4327 reconstructs 6052 the sensor signal data from the primary frequency employing the higher harmonics to derive blood pressure values.

With continued reference to FIG. 38D, in one aspect, according to the method 6000, the sensing-signal circuit 4327 implements 6054 a Kalman and particle filter model based on the received sensor data. The signal-sensing circuit 4327 processes 6056 the sensor data subjected to the Kalman and particle filters to isolate pulse-waveform data from other periodic signals and other artifacts due to electronic noise and motion. The signal-sensing circuit 4327 isolates 6058 the pulse-waveform data to extract blood pressure values.

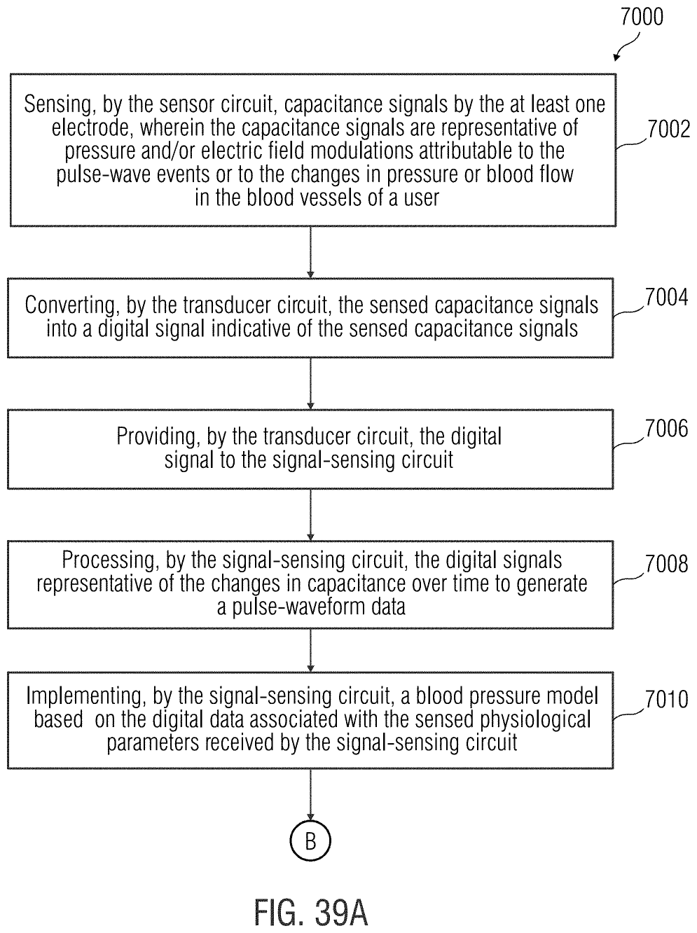

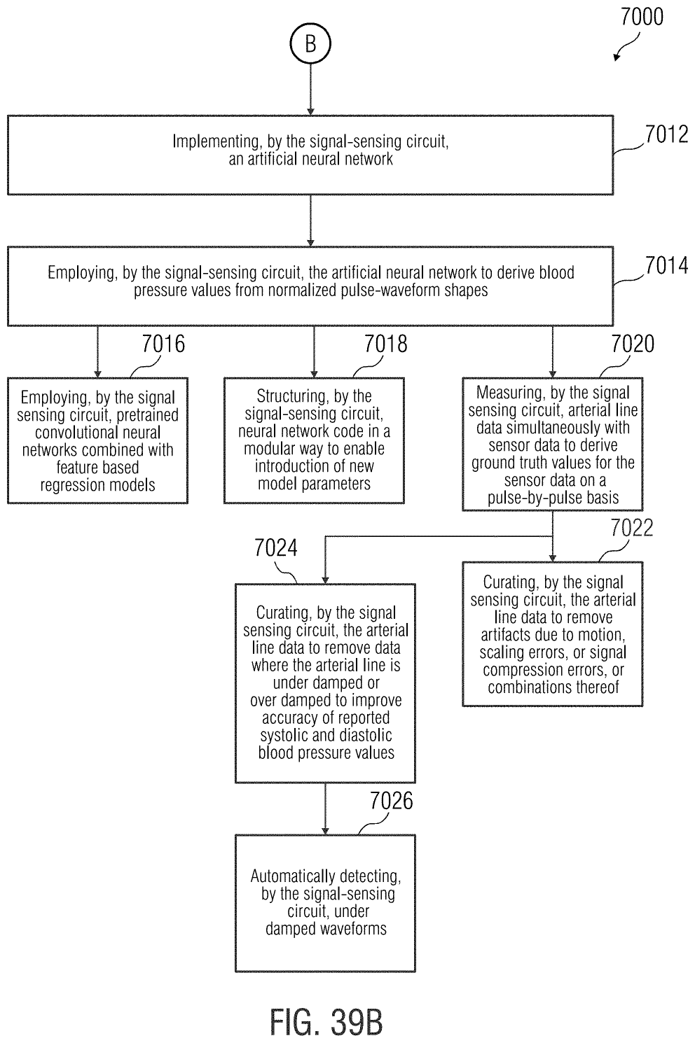

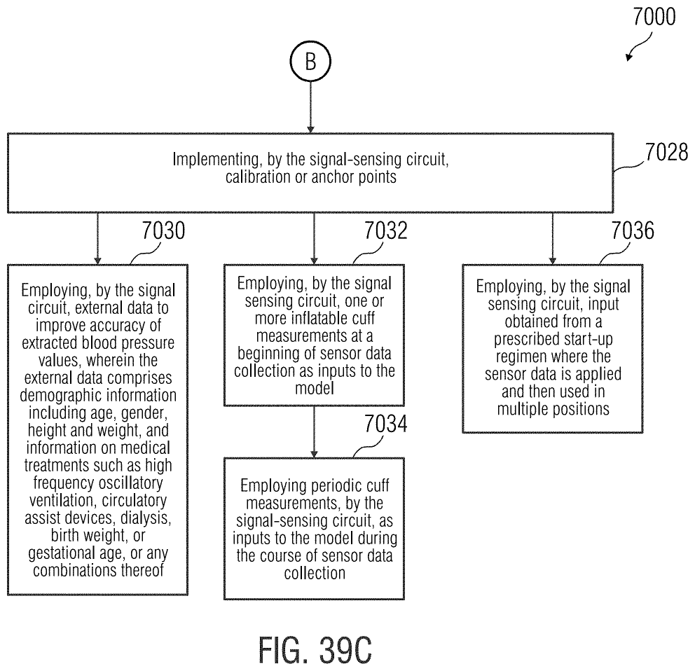

FIGS. 39A-39C illustrates a method 7000 for measuring and processing one or more physiological parameters, in accordance with at least one aspect of the present disclosure. The method 7000 comprises measuring and processing one or more physiological parameters via a wearable apparatus, such as the sensor band 2002, comprising a sensor circuit 4324 comprising at least one electrode 100-1300 placed near or onto the skin of a user, a transducer circuit 4326 to receive signals from the sensor circuit 4324 and to convert the signals to digital signals and provide the digital signals to the signal-sensing circuit 4327 to process the digital signals.

With reference to FIG. 39A, in one aspect, according to the method 7000, the sensor circuit 4324 senses 7002 capacitance signal changes between the electrode 100-1300 and the skin of a user, wherein the capacitance signal changes are representative of pressure and/or electric field modulations attributable to the pulse-wave events or to the changes in pressure or blood flow in the blood vessels (e.g., hemodynamics). The transducer circuit 4326 converts the sensed capacitance signals into a digital signal indicative of the sensed 7002 capacitance signal changes and/or pressure that is provided 7004 to the signal-sensing circuit 4327 for digital signal processing and/or communication, for example. Due to pulse-wave events, the distance between the skin of the user and the electrode 100-1300 can change and/or the electric field distribution around the blood vessels can change, resulting in a relative change in capacitance as measured using the sensor circuit 4324. The signal-sensing circuit 4327 processes 7006 the digital signals representative of the changes in capacitance over time and generates and/or determines a pulse-waveform. The signal-sensing circuit 4327 implements 7008 one or more blood pressure models based on the digital data associated with the sensed physiological parameters received by the signal-sensing circuit 4327.

With reference to FIG. 39B, in one aspect, according to the method 7000, the signal-sensing circuit 4327 implements 7012 an artificial neural network and employs 7014 the artificial neural network to derive blood pressure and/or other hemodynamic values from normalized pulse-waveform shapes. The signal-sensing circuit 4327 employs 7016 pre-trained convolutional neural networks combined with feature-based regression models. The signal-sensing circuit 4327 structures 7018 neural network code in a modular way to enable introduction of new model parameters. In some cases, the signal-sensing circuit 4327 also may be configured to measure 7020 arterial line data simultaneously with sensor data to derive ground truth values for the sensor data on a pulse-by-pulse basis. The signal-sensing circuit 4327 curates 7022 the pulse waveform data to remove artifacts due to motion, scaling errors, or signal compression errors, or combinations thereof and curates 7024 the arterial line data to remove data where the arterial line is underdamped or overdamped to improve accuracy of reported systolic and diastolic blood pressure values 7026 The signal-sensing circuit 4327 also may be configured to convert the digital data received and processed by 7012 into an [analog] output that can be displayed on a bedside monitor and inputted into the hospital's electronic medical records in the same manner as data from an arterial line transducer.

With reference to FIG. 39C, in one aspect, according to the method 7000, the signal-sensing circuit 4327 implements 7028 calibration or anchor points and/or employs 7030 external data to improve accuracy of extracted blood pressure values. The external data comprises demographic information such as age, gender, height and weight, and information on medical treatments such as high frequency oscillatory ventilation, circulatory assist devices, dialysis, birthweight, or gestational age, or any combinations thereof. The signal-sensing circuit 4327 employs 7032 one or more inflatable cuff measurements at a beginning of sensor data collection as inputs to the model, employs 7034 periodic cuff measurements as inputs to the model during the course of sensor data collection, and/or employs 7036 input obtained from a prescribed start-up regimen where the sensor data is applied and then used in multiple positions.

### EXAMPLES

Various aspects of the subject matter described herein are set out in the following numbered examples.

Example 1. A proximity sensor, comprising: a first dielectric layer comprising an inner surface and an outer surface: an electrically conductive layer positioned proximate to one of the inner surface or the outer surface of the first dielectric layer; and an electrode comprising an outer surface, the outer surface of the electrode positioned proximate the inner surface of the first dielectric layer, wherein the outer surface of the electrode and the electrically conductive layer define a gap.

Example 2. The proximity sensor of Example 1, wherein the electrically conductive layer is positioned proximate the outer surface of the first dielectric layer; and the electrode positioned proximate the inner surface of the first dielectric layer, the electrode comprising an inner surface and an outer surface, the outer surface of the electrode positioned proximate the inner surface of the first dielectric layer, wherein the outer surface of the electrode and the electrically conductive layer define a gap.

Example 3. The proximity sensor of any one of Examples 1-2, wherein the first dielectric layer has a thickness of up to 150 μm.

Example 4. The proximity sensor of any one of Examples 1-3, further comprising a substrate and a second dielectric layer, the second dielectric layer comprising an inner surface and an outer surface, wherein the second dielectric layer is disposed between the inner surface of the electrode and the substrate.

Example 5. The proximity sensor of Example 4, further comprising an adhesive layer positioned between the substrate and the inner layer of the second dielectric layer.

Example 6. The proximity sensor of any one of Examples 1-5, further comprising: an electrically conductive element electrically coupled to the electrode to provide an electrical connection between the electrode and an electronic circuit; and an adhesive layer comprising an inner surface and outer surface, the adhesive layer disposed between the inner surface of the first dielectric layer and the electrically conductive element.

Example 7. The proximity sensor of Example 6, wherein the electrically conductive element is disposed proximate to the inner surface of the first dielectric layer or proximate to the outer surface of the second dielectric layer.

Example 8. The proximity sensor of any one of Examples 6-7, further comprising a dielectric foam or double sided tape disposed between the inner surface of the first dielectric layer and the outer surface of the adhesive layer.

Example 9. The proximity sensor of any one of Examples 1-8, wherein the electrically conductive layer is positioned proximate the inner surface of the first dielectric layer; and further comprising a second dielectric layer disposed between the electrode and the electrically conductive layer, wherein the outer surface of the electrode and the electrically conductive layer define a gap; wherein the dielectric layers may be floating or fastened to other components comprising the proximity sensor to control the gap.

Example 10. The proximity sensor of Example 9, wherein the second dielectric layer has a thickness of up to 150 μm.

Example 11. The proximity sensor of any one of Examples 9-10, wherein the second dielectric layer has a thickness less than 5 μm.

Example 12. The proximity sensor of any one of Examples 9-11, wherein the second dielectric layer has a thickness less than 3 μm.

Example 13. The proximity sensor of any one of Examples 9-12, wherein the second dielectric layer has a textured surface.

Example 14. The proximity sensor of any one of Examples 9-13, further comprising a substrate and a third dielectric layer disposed between the electrode and the substrate.

Example 15. The proximity sensor of Example 14, further comprising a polymer layer disposed between the third dielectric layer and the substrate.

Example 16. The proximity sensor of Example 15, further comprising an adhesive layer positioned between the substrate and the polymer layer.

Example 17. A proximity sensor, comprising: a first dielectric layer comprising an inner surface and an outer surface; an electrically conductive layer positioned proximate to one of the inner surface or the outer surface of the first dielectric layer; a sensing electrode positioned proximate the inner surface of the first dielectric layer, the sensing electrode comprising an inner surface and an outer surface, the outer surface of the sensing electrode positioned proximate the inner surface of the first dielectric layer, wherein the outer surface of the sensing electrode and the electrically conductive layer define a gap; a reference electrode disposed relative to the sensing electrode, the reference electrode positioned proximate the inner surface of the first dielectric layer, the reference electrode comprising an inner surface and an outer surface, the outer surface of the reference electrode positioned proximate the inner surface of the first dielectric layer, wherein the outer surface of the reference electrode and the electrically conductive layer define a gap.

Example 18. The proximity sensor of Example 17, wherein an adhesive layer is disposed between the inner surface of the first dielectric layer and the outer surface of the reference electrode.

Example 19. The proximity sensor of any one of Examples 17-18, wherein the reference electrode is disposed laterally relative to the sensing electrode.

Example 20. The proximity sensor of any one of Examples 17-19, wherein the reference electrode is stacked relative to the sensing electrode.

Example 21. The proximity sensor of any one of Examples 17-20, wherein the sensing electrode and reference electrode are mechanically isolated.

Example 22. The proximity sensor of any one of Examples 17-21, further comprising: a first substrate; a second substrate; a third dielectric layer disposed between the sensing electrode and the first substrate; and a fourth dielectric layer disposed between the reference electrode and the reference electrode.

Example 23. The proximity sensor of Example 22, further comprising: a first adhesive layer positioned between the first substrate and the third dielectric layer; and a second adhesive layer positioned between the second substrate and the fourth dielectric layer.

Example 24. The proximity sensor of any one of Examples 22-23, further comprising a fifth dielectric layer disposed between the reference electrode and the first dielectric layer.

Example 25. The proximity sensor of Example 24, further comprising a sixth dielectric layer disposed between the sensing electrode and the first dielectric layer.

Example 26. The proximity sensor of any one of Examples 22-25, further comprising a cover film disposed over the first and second substrates.

Example 27. The proximity sensor of any one of Examples 22-26, wherein the first and second substrates are located along the same plane.

Example 28. The proximity sensor of any one of Examples 22-27, wherein the first and second substrates are located along different planes, wherein the proximity sensor further comprises: a mounting structure; and a foam layer disposed between the first and second substrates to provide conformity and to ensure that both the reference and sensing electrodes have similar contact planes, wherein the foam layer portion between the first substrate and the mounting structure has a first thickness and the foam layer between the second substrate and the mounting structure has a second thickness that is different from the first thickness.

Example 29. The proximity sensor of any one of Examples 17-28, further comprising: a foam layer, wherein the sensing electrode and the reference electrode are positioned on opposite sides of the foam layer.

Example 30. The proximity sensor of any one of Examples 17-29, further comprising a sealant layer disposed over the sensing surface.

Example 31. The proximity sensor of any one of Examples 29-30, further comprising a mounting structure positioned on the same side of the foam layer as the reference electrode.

Example 32. A proximity sensor module, comprising: a sensor element substrate, wherein the sensor element comprises any one of the proximity sensors defined in any one of Examples 1-31; at least one electrically conductive electrode lead disposed on the sensor element substrate; at least one elastically-deformable electrically-conductive feature disposed on the at least one electrically conductive electrode lead; an electronics module; at least one electrically conductive pad disposed on the electronics module, the at least one electrically conductive pad positioned to make an electrical connection between the at least one electrically conductive lead and the at least one electrically conductive pad through the at least one elastically-deformable electrically-conductive feature.

Example 33. The proximity sensor module of Example 32, further comprising: a plurality of electrically conductive leads; a plurality of elastically-deformable electrically-conductive features disposed on the plurality of electrically conductive electrode leads; and a plurality of electrically conductive pads disposed on the electronics module, the plurality of electrically conductive pads positioned to make electrical connections between the plurality of electrically conductive leads and the plurality of electrically conductive pads through the plurality of elastically-deformable electrically-conductive features.

Example 34. The proximity sensor module of any one of Examples 32-33, wherein the sensor element substrate is embossed.

Example 35. The proximity sensor module of Example 34, further comprising a compliant substrate disposed below the embossed sensor element substrate to structurally support the embossed sensor element substrate.

Example 38. The proximity sensor module of any one of Examples 1-35, further comprising a clamshell housing configured to receive the electronics module.

Example 37. The proximity sensor module of Example 36, further comprising a fastener disposed between the sensor element and the clamshell housing.

Example 38. The proximity sensor module of Example 37, wherein the fastener comprises a hook and loop fastener.

Example 39. A circuit for measuring physiological parameters, the circuit comprising: a sensor circuit comprising a sensor element substrate comprising any one of the proximity sensors defined in any one of Examples 1-31 comprising at least one electrode, wherein the sensor circuit is configured to monitor a capacitance signal between the at least one electrode and the skin of a user, wherein the capacitance signal represents pressure and/or electric field modulations attributable to pulse-wave events or to changes in motion, pressure or blood flow in blood vessels of the user or to movement of parts of the body of the user; a transducer circuit coupled to the sensor circuit, wherein the transducer circuit is configured to convert the monitored capacitance signal into a digital signal indicative of the monitored capacitance signal; and a signal-sensing circuit configured to receive the digital signal and determine at least one physiological parameter associated with the user.

Example 40. The circuit of Example 39, wherein the physiological parameters comprise blood pressure, systolic, diastolic, mean arterial pressure, or pulse pressure, respiration rate, or combinations thereof, and their variabilities, both as time series values and as trends.

Example 41. The circuit of any one of Examples 39-40, wherein the signal-sensing circuit is configured to measure a pulse wave or a heart beat as a substitute for Doppler measurements.

Example 42. The circuit of any one of Examples 39-41, wherein the signal-sensing circuit is configured to monitor blood pressure and heart rate trends.

Example 43. The circuit of any one of Examples 39-42, wherein the electronic circuit is configured to measure multiple pulse points and provide circulatory comparisons.

Example 44. The circuit of any one of Examples 39-43, wherein the signal-sensing circuit is configured to determine complications or efficacy of operative procedures through circulatory time analysis using trends in blood pressure or pulse height values or changes in pulse-waveform shapes.

Example 45. A circuit for measuring physiological parameters, the circuit comprising: a sensor circuit comprising a sensor element substrate comprising any one of the proximity sensors defined in any one of Examples 1-31 comprising at least one electrode, wherein the sensor circuit is configured to monitor a capacitance signal between the at least one electrode and the skin of a user, wherein the capacitance signal represents pressure and/or electric field modulations attributable to pulse-wave events or to changes in pressure or blood flow in blood vessels of the user; a transducer circuit coupled to the sensor circuit, wherein the transducer circuit is configured to convert the monitored capacitance signal into a digital signal indicative of the monitored capacitance signal; and a signal-sensing circuit configured to implement quality models.

Example 46. The circuit of Example 45, wherein the signal-sensing circuit is configured to implement a regression coefficient model as a metric of sensor data quality.

Example 47. The circuit of any one of Examples 45-46, wherein the signal-sensing circuit is configured to employ a neural network trained on the sensor data with regression coefficients as ground truth values, wherein the network is employed to predict likelihood that subsequent sensor data correlates to arterial line data, wherein the likelihood is employed as a quality metric to filter sensor data that is be fed into algorithms to extract blood pressure values from sensor pulse-waveform data.

Example 48. The circuit of Example 47, wherein the signal-sensing circuit is configured to employ the likelihood to estimate a confidence level of the extracted blood pressure values.

Example 49. The circuit of any one of Examples 45-48, wherein the signal-sensing circuit is configured to implement a pulse-waveform quality model.

Example 50. The circuit of Example 49, wherein the signal-sensing circuit is configured to train on quality ratings from a rubric based on features of pulse-waveforms such as resolution of secondary peaks, signal-to-noise levels, lack of baseline variations, or motion artifacts, or combinations thereof.

Example 51. The circuit of Example 50, wherein the signal-sensing circuit is configured to visually rate pulse-waveforms and train a convolutional neural network on the pulse-waveform data with the ratings used as ground truth values.

Example 52. The circuit of any one of any one of Examples 49-51, wherein the signal-sensing circuit is configured to provide quality ratings for subsequent sensor data to filter sensor data for use to extract blood pressure values or to estimate a confidence level for the extracted values.

Example 53. The circuit of any one of Examples 49-52, wherein the signal-sensing circuit is configured to classify waveforms into different canonical shapes to identify a class of waveform shapes for each new pulse-waveform to determine whether a blood pressure value can be extracted from that pulse-waveform and/or which model to use.

Example 54. The circuit of any one of Examples 45-53, wherein the signal-sensing circuit is configured to implement a signal to noise ratio model.

Example 55. The circuit module of Example 54, wherein the signal-sensing circuit is configured to implement a quality model based on Fourier filtering based on the Fourier Transform of the sensor data.

Example 56. The circuit of any one of Examples 54-55, wherein the signal sensing circuit is configured to implement bandstop filters to remove periodic noise such as breathing modes at lower frequencies and oscillatory ventilation noise at higher frequencies.

Example 57. The circuit of any one of Examples 45-56, wherein the signal-sensing circuit is configured to calculate signal power by identifying a primary frequency of a heart rate and integrating over a peak of the signal power along with a number of higher harmonics of the signal data.

Example 58. The circuit of Example 57, wherein the signal-sensing circuit is configured to integrate remaining data to determine a noise power value.

Example 59. The circuit of Example 58, wherein the signal sensing circuit is configured to calculate a ratio of signal power to noise power to yield a metric indicative of a general quality of the sensor data.

Example 60. The circuit of any one of Examples 58-59, wherein the signal-sensing circuit is configured to calculate the ratio of signal power to noise power with a sliding data window to determine a signal-to-noise ratio (SNR) as a function of time to filter/select the sensor data for further processing.

Example 61. The circuit of Example 60, wherein the signal-sensing circuit is configured to reconstruct the signal data from the primary frequency and employ the higher harmonics to derive blood pressure values.

Example 62. The circuit of any one of Examples 45-61, wherein the signal-sensing circuit is configured to implement a Kalman and particle filter model.

Example 63. The circuit of Example 62, wherein the signal-sensing circuit is configured to process sensor data subjected to Kalman and particle filters to isolate pulse-waveform data from other periodic signals and other artifacts due to electronic noise and motion.

Example 64. The circuit of Example 63, wherein the signal-sensing circuit is configured to isolate pulse-waveform data to extract blood pressure values.

Example 65. A circuit for measuring physiological parameters, the circuit comprising: a sensor circuit comprising a sensor element substrate comprising any one of the proximity sensors defined in any one of Examples 1-31 comprising at least one electrode, wherein the sensor circuit is configured to monitor a capacitance signal between the at least one electrode and the skin of a user, wherein the capacitance signal represents motion, pressure and/or electric field modulations attributable to pulse-wave events, to changes in pressure or blood flow in blood vessels of the user, or to movements of parts of the body of the user; a transducer circuit coupled to the sensor circuit, wherein the transducer circuit is configured to convert the monitored capacitance signal into a digital signal indicative of the monitored capacitance signal; and a signal-sensing circuit configured to implement blood pressure and other hemodynamic and physiological models.

Example 66. The circuit of Example 65, wherein the signal-sensing circuit is configured to convert the capacitance signal to a format that can be displayed on an external monitor and/or processed and stored on an external data system.

Example 67. The circuit of any one of Examples 65-66, wherein the signal-sensing circuit is configured to implement an artificial neural network.

Example 68. The circuit of Example 67, wherein the signal-sensing circuit is configured to employ the artificial neural network (NN) to derive blood pressure values from normalized pulse-waveform shapes.

Example 69. The circuit of Example 68, wherein the signal-sensing circuit is configured to employ pretrained convolutional neural networks combined with feature-based regression models.

Example 70. The circuit of any one of Examples 68-69, wherein the signal-sensing circuit is configured to structure NN code in a modular way to enable introduction of new model parameters.

Example 71. The circuit of any one of Examples 68-70, wherein the signal-sensing circuit is configured to curate the pulse waveform data to remove artifacts due to motion, scaling errors, or signal compression errors, or combinations thereof.

Example 72. The circuit of any one of Examples 65-71, wherein the signal-sensing circuit is configured to implement calibration or anchor points.

Example 73. The circuit of Example 72, wherein the signal-sensing circuit is configured to employ external data to improve accuracy of extracted blood pressure values.

Example 74. The circuit of Example 73, wherein the external data comprises demographic information such as age, gender, height and weight, and information on medical treatments such as high frequency oscillatory ventilation, circulatory assist devices, dialysis, birthweight, or gestational age, or any combinations thereof.

Example 75. The circuit of any one of Examples claim 72-74, wherein the signal-sensing circuit is configured to employ one or more inflatable cuff measurements at a beginning of sensor data collection as inputs to the model.

Example 76. The circuit of Example 75, wherein the signal-sensing circuit is configured to employ periodic cuff measurements as inputs to the model during the course of sensor data collection.

Example 77. The circuit of any one of Examples 72-76, wherein the signal-sensing circuit is configured to employ input obtained from a prescribed start-up regimen where the sensor is applied and then used in multiple positions.

Example 78. A method for hemodynamic monitoring via a wearable apparatus comprising a sensor circuit comprising at least one electrode, a transducer circuit to receive signals from the sensor circuit and to convert the signals to digital signals and provide the digital signals to a signal-sensing circuit to process the digital signals, the method comprising: sensing, by the sensor circuit, capacitance signals by the at least one electrode, wherein the capacitance signals are representative of pressure and/or electric field modulations attributable to the pulse-wave events or to the changes in pressure or blood flow in the blood vessels of a user; converting, by the transducer circuit, the sensed capacitance signals into a digital signal indicative of the sensed capacitance signals; providing, by the transducer circuit, the digital signal to the signal-sensing circuit; processing, by the signal-sensing circuit, the digital signals representative of the changes in capacitance over time to generate a pulse-waveform data; correlating, by the signal-sensing circuit, the pulse-waveform data with various hemodynamic parameters; processing, by the signal-sensing circuit, the pulse-waveform data; and determining, by the signal-sensing circuit, a hemodynamic parameter based on the pulse-waveform data.

Example 79. The method of Example 78, further comprising reducing motion artifacts with an accessory device.

Example 80. The method of Example 79, wherein the accessory device comprises vibration damping material to dampen vibrations or motion artifacts.

Example 81. A method for measuring and processing one or more physiological parameters via a wearable apparatus comprising a sensor circuit comprising at least one electrode placed near or onto the skin of a user, a transducer circuit to receive signals from the sensor circuit and to convert the signals to digital signals and provide the digital signals to a signal-sensing circuit to process the digital signals, the method comprising: sensing, by the sensor circuit, capacitance signals by the at least one electrode, wherein the capacitance signals are representative of pressure and/or electric field modulations attributable to the pulse-wave events or to the changes in pressure or blood flow in the blood vessels of a user; converting, by the transducer circuit, the sensed capacitance signals into a digital signal indicative of the sensed capacitance signals; providing, by the transducer circuit, the digital signal to the signal-sensing circuit; processing, by the signal-sensing circuit, the digital signals representative of the changes in capacitance over time to generate a pulse-waveform data; correlating, by the signal-sensing circuit, the pulse-waveform data with various hemodynamic parameters; processing, by the signal-sensing circuit, the pulse-waveform data; and implementing, by the signal-sensing circuit, a regression coefficient model based on the digital data associated with the sensed physiological parameters received by the signal-sensing circuit.

Example 82. The method of Example 81, comprising determining, by the signal-sensing circuit, regression coefficients between sensor data and reference arterial line data using pulse-by-pulse synchronized arterial and sensor data.

Example 83. The method of Example 82, comprising employing, by the signal-sensing circuit, the regression coefficients as a metric of sensor data quality.

Example 84. The method of any one of Examples 82-83, comprising: employing, by the signal-sensing circuit, a neural network trained on the sensor data with the regression coefficients as ground truth values; predicting, by the signal-sensing circuit, a likelihood that subsequent sensor data correlates to arterial line data; employing, by the signal-sensing circuit, the likelihood as a quality metric to filter sensor data to extract blood pressure values from the pulse-waveform data; and estimating, by the signal-sensing circuit, a confidence level of the extracted blood pressure values based on the likelihood.

Example 85. The method of any one of Examples 81-84, comprising implementing, by the signal-sensing circuit, a pulse-waveform quality model based on the received sensor data.

Example 86. The method of Example 85, comprising training, by the signal-sensing circuit, on quality ratings from a rubric based on features of pulse-waveforms; visually rating, by the signal-sensing circuit, the pulse-waveform data; and training, by the signal-sensing circuit, a convolutional neural network on the pulse-waveform data with the ratings used as ground truth values.

Example 87. The method of any one of Examples 85-86, comprising providing, by the signal-sensing circuit, quality ratings for subsequent sensor data to filter sensor data to extract blood pressure values or estimate a confidence level for the extracted values.

Example 88. The method of claim any one of Examples 85-87, comprising classifying, by the signal-sensing circuit, the pulse-waveform data into different canonical shapes to identify a class of waveform shapes for each new pulse-waveform to determine whether a blood pressure value can be extracted from that pulse-waveform and/or which model to use.

Example 89. The method of any one of Examples 81-88, comprising implementing, by the signal-sensing circuit, a signal-to-noise ratio model based on the received sensor data.

Example 90. The method of Example 89, comprising implementing, by the signal-sensing circuit, a quality model based on a Fourier filter based on the Fourier Transform of the received sensor data.

Example 91. The method of any one of Examples 89-90, comprising implementing, by the signal-sensing circuit, a bandstop filter to remove periodic noise such as breathing modes at lower frequencies and oscillatory ventilation noise at higher frequencies.

Example 92. The method of any one of Examples 89-91, comprising: calculating, by the signal-sensing circuit, sensor data signal power by identifying a primary frequency associated with a heart rate; and integrating, by the signal-sensing circuit, over a peak of the signal power along with a number of higher harmonics of the signal data.

Example 93. The method of Example 92, comprising: integrating, the signal-sensing circuit, the remaining data to determine a noise power value; and calculating, by the signal-sensing circuit, a ratio of signal power to noise power to yield a metric indicative of a general quality sensor data.

Example 94. The method of any one of any one of Examples 92-93, comprising: calculating, by the signal-sensing circuit, a ratio of signal power to noise power with a sliding data window to determine a signal-to-noise ratio (SNR) as a function of time to filter/select the received sensor data for further processing; and reconstructing, by the signal-sensing circuit, the sensor signal data from the primary frequency employing the higher harmonics to derive blood pressure values.

Example 95. The method of any one of Examples 81-94, comprising: implementing, by the signal-sensing circuit, a Kalman and particle filter model based on the received sensor data; processing, by the signal-sensing circuit, the sensor data subjected to the Kalman and particle filters to isolate the pulse-waveform data from other periodic signals and other artifacts due to electronic noise and motion; and isolating, by the signal-sensing circuit, the pulse-waveform data to extract blood pressure values.

Example 96. A method for measuring and processing one or more physiological parameters via a wearable apparatus comprising a sensor circuit comprising at least one electrode placed near or onto the skin of a user, a transducer circuit to receive signals from the sensor circuit and to convert the signals to digital signals and provide the digital signals to a signal-sensing circuit to process the digital signals, the method comprising: sensing, by the sensor circuit, capacitance signals by the at least one electrode, wherein the capacitance signals are representative of motion, pressure and/or electric field modulations attributable to pulse-wave events, to changes in pressure or blood flow in blood vessels of the user, or to movements of parts of the body of the user; converting, by the transducer circuit, the sensed capacitance signals into a digital signal indicative of the sensed capacitance signals; providing, by the transducer circuit, the digital signal to the signal-sensing circuit: processing, by the signal-sensing circuit, the digital signals representative of the changes in capacitance over time to generate a pulse-waveform data; correlating, by the signal-sensing circuit, the pulse-waveform data with various hemodynamic parameters; processing, by the signal-sensing circuit, the pulse-waveform data; and implementing, by the signal-sensing circuit, a model based on the digital data associated with the sensed physiological parameters received by the signal-sensing circuit.

Example 97. The method of Example 96, comprising: implementing, by the signal-sensing circuit, an artificial neural network; and employing, by the signal-sensing circuit, the artificial neural network to derive blood pressure and other hemodynamic values from normalized pulse-waveform shapes.

Example 98. The method of Example 97, comprising employing, by the signal-sensing circuit, pre-trained convolutional neural networks combined with feature-based regression models.

Example 99. The method of any one of Examples 97-98, comprising structuring, by the signal-sensing circuit, neural network code in a modular way to enable introduction of new model parameters.

Example 100. The method of any one of Examples 97-99, comprising measuring, by the signal-sensing circuit, arterial line data simultaneously with sensor data to derive ground truth values for the sensor data on a pulse-by-pulse basis.

Example 101. The method of Example 100, comprising curating, by the signal-sensing circuit, the arterial line data to remove artifacts due to motion, scaling errors, or signal compression errors, or combinations thereof.

Example 102. The method of any one of Examples 100-101, comprising curating, by the signal-sensing circuit, the arterial line data to remove data where the arterial line is underdamped or overdamped to improve accuracy of reported systolic and diastolic blood pressure values.

Example 103. The method of Example 102, comprising automatically detecting, by the signal-sensing circuit, underdamped waveforms.

Example 104. The method of any one of Examples 96-103, comprising implementing, by the signal-sensing circuit, calibration or anchor points.

Example 105. The method of Example 104, comprising employing, by the signal circuit, external data to improve accuracy of extracted blood pressure values, wherein the external data comprises demographic information including age, gender, height and weight, and information on medical treatments such as high frequency oscillatory ventilation, circulatory assist devices, dialysis, birthweight, or gestational age, or any combinations thereof.

Example 106. The method of any one of Examples 104-105, comprising: employing, by the signal-sensing circuit, one or more inflatable cuff measurements at a beginning of sensor data collection as inputs to the model; and employing periodic cuff measurements, by the signal-sensing circuit, as inputs to the model during the course of sensor data collection.

Example 107. The method of any one of Examples 104-106, comprising employing, by the signal-sensing circuit, input obtained from a prescribed start-up regimen where the sensor data is applied and then used in multiple positions.

Terms to exemplify orientation and direction, such as upper/lower, left/right, top/bottom, up/down, above/below, vertical, horizontal, and perpendicular, may be used herein to refer to relative positions of elements as shown in the figures. Similarly, as heating and cooling are relative terms of art, it is appreciated that a heating source and a cooling source can be synonymous considering that the direction of temperature change can be controlled according to the desired temperature change. It should be understood that the terminology is used for notational convenience only and that in actual use the disclosed structures may be oriented different from the orientation shown in the figures. Thus, the terms should not be construed in a limiting manner.

It may also be helpful to appreciate the context/meaning of the following terms: the term “electrode” refers to or includes a conductive conductor; the term “sensor circuit” refers to or includes to a circuit including the electrode and a connection to the transducer circuit (e.g., has a sensor connector for the electrode to be plug in or otherwise connected to the transducer circuit), and that detects or measures the capacitance values and/or changes in capacitance via the electrode and is used to output the same to the transducer circuit; the sensor circuit may additionally include various other elements, such as those illustrated by FIGS. 29A-29B and 30A-30B, for example, the sensor circuit can include a multilayer construction that includes the electrode and various dielectric and conductive layers; the term “transducer circuit” refers to or includes circuitry that converts variations in a physical quality, such as changes in capacitance as provided by the sensor circuit, into electrical signals; for example, the transducer circuit can include a capacitance-to-digital converter; the term “pulse-wave events” refers to or includes hemodynamic responses and/or attributes caused by and/or indicative of heart beats (e.g., contraction of heart muscles) (e.g., heart beats or sounds, changes in blood pressure or blood flow velocity, etc.); the term “pulse-waveform”refers to or includes a signal or wave shape generated by the pulse-wave events; example pulse-waveforms include an arterial pulse-waveform, e.g., a wave shape generated by the heart when the heart contracts and the wave travels along the arterial walls of the arterial tree; the term “electrical-signal sensing circuit” refers to or includes a circuit that is used to sense the hemodynamic or pulse-wave events using the electrical signals from the transducer circuit: an example electrical-signal sensing circuit includes a microcontroller or other processing circuitry and an example transducer circuit includes a capacitance-to-digital converter, although aspects are not so limited: the term “communication circuit” refers to or includes a circuit that outputs data to other external circuitry, which can include a wireless or a wired communication; an example communication circuit includes a transceiver, although aspects are not so limited; the terms “hemodynamic” or “hemodynamic parameters” refers to or includes parameters relating to the flow of blood within the organs, blood vessels, and tissues of the body; example hemodynamic or hemodynamic parameters can include diastolic blood pressure, systolic blood pressure, arterial stiffness, and blood volume, among other parameters.

Various blocks, modules or other circuits may be implemented to carry out one or more of the operations and activities described herein and/or shown in the figures. For example, processes such as heating, etching, and deposition can be automated through the use of various circuits and associated machines. In these contexts, various depicted functions can be implemented using a circuit that carries out one or more of these or related operations/activities. In various aspects, a hard-wired control block can be used to minimize the area for such an implementation in case a limited flexibility is sufficient. Alternatively and/or in addition, in certain of the above-discussed aspects, one or more modules are discrete logic circuits or programmable logic circuits configured and arranged for implementing these operations/activities.

As examples, the Specification describes and/or illustrates aspects useful for implementing the claimed disclosure by way of various circuits or circuitry which may be illustrated as or using terms such as blocks, modules, device, system, and/or other circuit-type depictions. Such circuits or circuitry are used together with other elements (wristbands, external processing circuitry and the like) to exemplify how certain aspects may be carried out in the form or structures, steps, functions, operations, activities, etc. For example, in certain of the above-discussed aspects, one or more illustrated items in this context represent circuits (e.g., discrete logic circuitry or (semi-) programmable circuits) configured and arranged for implementing these operations/activities, as may be carried out in the approaches shown in the slides. In certain aspects, such illustrated items represent one or more computer circuitry (e.g., microcomputer or other CPU) which is understood to include memory circuitry that stores code (program to be executed as a set/sets of instructions) for performing a basic algorithm (e.g., monitoring pressure differentials and/or capacitance changes attributable to pulse-wave events), and/or involving determining hemodynamic parameters, and/or a more complex process/algorithm as would be appreciated from known literature describing such specific-parameter sensing. Such processes/algorithms would be specifically implemented to perform the related steps, functions, operations, activities, as appropriate for the specific application. The specification may also make reference to an adjective that does not connote any attribute of the structure (“first [type of structure]” and “second [type of structure]”) in which case the adjective is merely used for English-language antecedence to differentiate one such similarly-named structure from another similarly-named structure (e.g., “first electrode . . . ” is interpreted as “an electrode . . . ”).

Based upon the above discussion and illustrations, those skilled in the art will readily recognize that various modifications and changes may be made to the various aspects without strictly following the exemplary aspects and applications illustrated and described herein. For example, methods as exemplified in the Figures may involve steps carried out in various orders, with one or more aspects of the aspects herein retained, or may involve fewer or more steps. Such modifications do not depart from the true spirit and scope of various aspects of the disclosure, including aspects set forth in the claims.

1. A proximity sensor, comprising:
a first dielectric layer comprising an inner surface and an outer surface;
an electrically conductive layer positioned proximate to one of the inner surface or the outer surface of the first dielectric layer; and
an electrode comprising an outer surface, the outer surface of the electrode positioned proximate the inner surface of the first dielectric layer, wherein the outer surface of the electrode and the electrically conductive layer define a gap.

2. The proximity sensor of claim 1, further comprising a foam layer.

3. The proximity sensor of claim 1, further comprising a sealant layer disposed over the sensing surface.

4. The proximity sensor of claim 1, wherein the electrically conductive layer is positioned proximate the inner surface of the first dielectric layer; and
further comprising a second dielectric layer disposed between the electrode and the electrically conductive layer, wherein the outer surface of the electrode and the electrically conductive layer define a gap.

5. The proximity sensor of claim 4, wherein the second dielectric layer has a thickness less than 3 μm.

6. The proximity sensor of claim 4, wherein the second dielectric layer has a textured surface.

7. A proximity sensor, comprising:
a first dielectric layer comprising an inner surface and an outer surface;
an electrically conductive layer positioned proximate to one of the inner surface or the outer surface of the first dielectric layer;
a sensing electrode positioned proximate the inner surface of the first dielectric layer, the sensing electrode comprising an inner surface and an outer surface, the outer surface of the sensing electrode positioned proximate the inner surface of the first dielectric layer, wherein the outer surface of the sensing electrode and the electrically conductive layer define a gap; and
a reference electrode disposed relative to the sensing electrode, the reference electrode positioned proximate the inner surface of the first dielectric layer, the reference electrode comprising an inner surface and an outer surface, the outer surface of the reference electrode positioned proximate the inner surface of the first dielectric layer, wherein the outer surface of the reference electrode and the electrically conductive layer define a gap.

8. The proximity sensor of claim 7, wherein the reference electrode is disposed laterally relative to the sensing electrode, stacked relative to the sensing electrode, or mechanically isolated from the sensing electrode.

9. The proximity sensor of claim 7, further comprising a fifth dielectric layer disposed between the reference electrode and the first dielectric layer.

10. The proximity sensor of claim 7, further comprising a sixth dielectric layer disposed between the sensing electrode and the first dielectric layer.

11. The proximity sensor of claim 7, further comprising:
a substrate layer, wherein the sensing electrode and the reference electrode are positioned on opposite sides of the substrate layer.

12. A proximity sensor module, comprising:
a sensor element substrate, wherein the sensor element substrate comprises a proximity sensor;
at least one electrically conductive electrode lead disposed on the sensor element substrate;
an electronics module;
at least one electrically conductive pad disposed on the electronics module;
at least one elastically-deformable electrically-conductive feature disposed on at least one of the at least one electrically conductive electrode lead or the at least one electrically conductive electrode pad, wherein the one elastically-deformable electrically-conductive feature is positioned to make an electrical connection between the at least one electrically conductive lead and the at least one electrically conductive pad through the at least one elastically-deformable electrically-conductive feature.

13. A circuit for measuring physiological parameters, the circuit comprising:
a sensor circuit comprising a sensor element substrate comprising a proximity sensor comprising at least one electrode, wherein the sensor circuit is configured to monitor a capacitance signal between the at least one electrode and the skin of a user, wherein the capacitance signal represents motion, pressure and/or electric field modulations attributable to pulse-wave events or to changes in pressure or blood flow in blood vessels of the user or to movement of parts of the body of the user;
a transducer circuit coupled to the sensor circuit, wherein the transducer circuit is configured to convert the monitored capacitance signal into a digital signal indicative of the monitored capacitance signal; and
a signal-sensing circuit configured to receive the digital signal and determine at least one physiological parameter associated with the user.

14. The circuit of claim 13, wherein the physiological parameters comprise blood pressure, systolic, diastolic, mean arterial pressure, pulse pressure, respiration rate, or combinations thereof, and their variabilities, or as time series values and as trends.

15. The circuit of claim 13, wherein the signal-sensing circuit is configured to provide quality ratings for subsequent sensor data to filter sensor data for use to extract blood pressure values or to estimate a confidence level for the extracted values.

16. A circuit for measuring physiological parameters, the circuit comprising:
a sensor circuit comprising a sensor element substrate comprising a proximity sensor comprising at least one electrode, wherein the sensor circuit is configured to monitor a capacitance signal between the at least one electrode and the skin of a user, wherein the capacitance signal represents motion, pressure and/or electric field modulations attributable to pulse-wave events, to changes in pressure or blood flow in blood vessels of the user, or to movements of parts of the body of the user;
a transducer circuit coupled to the sensor circuit, wherein the transducer circuit is configured to convert the monitored capacitance signal into a digital signal indicative of the monitored capacitance signal; and
a signal-sensing circuit configured to implement blood pressure or other hemodynamic or physiological models.

17. The circuit of claim 16, wherein the signal-sensing circuit is configured to convert the capacitance signal to a format that can be displayed on an external monitor and/or processed and stored on an external data system.

18. The circuit of claim 16, wherein the signal-sensing circuit is configured to employ input obtained from a prescribed start-up regimen where the sensor is applied and then used in multiple positions.

19. A method for hemodynamic monitoring via a wearable apparatus comprising a sensor circuit comprising at least one electrode, a transducer circuit to receive signals from the sensor circuit and to convert the signals to digital signals and provide the digital signals to a signal-sensing circuit to process the digital signals, the method comprising:
sensing, by the sensor circuit, capacitance signals by the at least one electrode, wherein the capacitance signals are representative of pressure and/or electric field modulations attributable to the pulse-wave events or to the changes in pressure or blood flow in the blood vessels of a user;
converting, by the transducer circuit, the sensed capacitance signals into a digital signal indicative of the sensed capacitance signals;
providing, by the transducer circuit, the digital signal to the signal-sensing circuit;
processing, by the signal-sensing circuit, the digital signals representative of the changes in capacitance over time to generate a pulse-waveform data;
correlating, by the signal-sensing circuit, the pulse-waveform data with various hemodynamic parameters;
processing, by the signal-sensing circuit, the pulse-waveform data; and
determining, by the signal-sensing circuit, a hemodynamic parameter based on the pulse-waveform data.

20. The method of claim 19, further comprising reducing motion artifacts with an accessory device.
